(19) United States
(12) Patent Application Publication
(10) Pub. No.: US 2024/0307420 A1
(43) Pub. Date: Sep. 19, 2024

(54) IMPROVED METHODS FOR THE USE OF PSYCHEDELICS

(71) Applicant: TRYP Therapeutics Inc., Kelowna (CA)

(72) Inventors: James GILLIGAN, Basking Ridge, NJ (US); Larry NORDER, Boulder, CO (US)

(73) Assignees: TRYP Therapeutics Inc., Kelowna, BC (CA); TRYP Therapeutics Inc., Kelowna, BC (CA)

(21) Appl. No.: 18/281,935

(22) PCT Filed: Mar. 15, 2022
(86) PCT No.: PCT/IB2022/052347

§ 371 Date: Sep. 13, 2023

Related U.S. Application Data
(60) Provisional application No. 63/161,070, filed on Mar. 15, 2021.

(51) Int. Cl. A61K 31/675; A61B 5/00; A61B 5/16; A61B 5/369; A61K 9/00; A61K 31/4045; A61P 25/04

(52) U.S. Cl. CPC A61K 31/675; A61B 5/165; A61B 5/369; A61B 5/4848; A61K 9/0019; A61K 31/4045; A61P 25/04

(57) ABSTRACT

Provided are improved methods for treating a psychological disorder in a subject comprising administering to the subject an amount of psilocybin or psilocin sufficient to induce a dissociative state in the subject less than 30 minutes after the administration; and thereafter maintaining the mean plasma concentration of the psychedelic at a predetermined value to maintain the dissociative state during a therapeutic window.

### CROSS-REFERENCE TO RELATED APPLICATIONS

This application claims priority from U.S. Provisional Application No. 63/161,070, filed Mar. 15, 2021, entitled “IMPROVED METHODS FOR THE USE OF PSYCHEDELICS,” the contents of which are incorporated by reference in their entirety.

### FIELD

The technology relates to formulations and improved methods for the use of psilocybin, psilocin and other psychedelic compounds in the treatment of psychological disorders.

### BACKGROUND

Psilocybin (4-phosphoroyloxy-N,N-dimethyltryptamine) is a tryptamine psychedelic that has similar effects to dimethyltryptamine (DMT), lysergic acid diethylamide (LSD) and mescaline, producing psychoactive effects. Psilocybin has been investigated as a treatment for certain mental health conditions. However, improved methods and compositions are needed to overcome challenges such as the potential for intoxication and difficulties in controlling the doses and pharmacokinetic parameters exist. Provided herein are methods and compositions that meet such needs.

### SUMMARY

Provided herein are methods of treating a psychological disorder in a subject that involves the administration of a psychedelic. In some of any of the provided embodiments, the psychedelic is psilocybin, psilocin, a co-crystal, a co-former, or a salt thereof, or a combination thereof.

In some of any of the provided embodiments, the methods involve administering to a subject having a psychological disorder an amount of a psychedelic sufficient to induce a dissociative state in the subject less than 30 minutes after administration; and thereafter maintaining the mean plasma concentration of the psychedelic at a predetermined value to maintain the dissociative state during a therapeutic window, wherein the psychedelic is psilocybin, psilocin, a co-crystal, a co-former, or a salt thereof, or a combination thereof.

In some of any embodiments, the methods also involve obtaining a non-invasive measurement of brain activity from the subject to determine when the subject enters the dissociative state.

Also provided herein are methods of inducing a dissociative state that involves the administration of a psychedelic. In some of any of the provided embodiments, the methods involve: administering to a subject having a psychological disorder, an amount of a psychedelic sufficient to induce the dissociative state in the subject; and obtaining a non-invasive measurement of brain activity from the subject to determine when the subject enters the dissociative state, wherein the psychedelic is psilocybin, psilocin, a co-crystal, a co-former, or a salt thereof, or a combination thereof.

Also provided herein are methods of inducing a dissociative state that involves administering to a subject having a psychological disorder, an amount of a psychedelic sufficient to induce the dissociative state in the subject; and obtaining a measurement of electroencephalography (EEG) from the subject to determine when the subject enters the dissociative state, wherein the psychedelic is psilocybin, psilocin, a co-crystal, a co-former, or a salt thereof, or a combination thereof.

Also provided herein are methods of determining a therapeutically effective dose of a psychedelic. In some of any of the provided embodiments, the methods involve administering to a subject having a psychological disorder, an amount of a psychedelic sufficient to induce a dissociative state in the subject; obtaining a non-invasive measurement of brain activity from the subject to determine if the subject enters the dissociative state; and determining the amount of the psychedelic as a therapeutically effective dose if the subject enters the dissociative state, wherein the psychedelic is psilocybin, psilocin, a co-crystal, a co-former, or a salt thereof, or a combination thereof.

Also provided herein are methods of maintaining a dissociative state in a subject with a psychological disorder. In some of any of the provided embodiments, the methods involve administering to the subject an amount of a psychedelic sufficient to induce the dissociative state in the subject less than 30 minutes after administration; and obtaining a measurement of electroencephalography (EEG) from the subject to determine when the subject enters the dissociative state; and thereafter maintaining a mean plasma concentration of the psychedelic at a predetermined value to maintain the dissociative state during a therapeutic window, wherein the psychedelic is psilocybin, psilocin, a co-crystal, a co-former, or a salt thereof, or a combination thereof.

Also provided herein are methods of treating a psychological disorder. In some of any of the provided embodiments, the methods involve performing any of the methods provided herein, and maintaining the mean plasma concentration of the psychedelic at a predetermined value to maintain the dissociative state during a therapeutic window; thereby treating the psychological disorder.

Also provided herein are methods of treating phantom limb pain in a subject. In some of any of the provided embodiments, the methods involve administering to a subject having phantom limb pain an amount of a psychedelic sufficient to induce a dissociative state in the subject, wherein the psychedelic is psilocybin, psilocin, a co-crystal, a co-former, or a salt thereof, or a combination thereof.

In some of any of the provided embodiments, the administration of the psychedelic is by intravenous administration.

In some of any embodiments, the dissociative state is induced in the subject within less than at or about 15, 30, 60, 90 or 120 minutes after administration, or a range defined by any of the foregoing. In some of any embodiments, the dissociative state is induced in the subject within less than at or about 30 minutes after administration.

In some of any embodiments, the methods also involve maintaining the mean plasma concentration of the psychedelic at a predetermined value to maintain the dissociative state during a therapeutic window.

In some of any embodiments, the non-invasive measurement of brain activity is selected from among electroencephalography (EEG), functional magnetic resonance imaging (fMRI), near-infrared spectroscopy (NIRS), magnetoencephalography (MEG), and optoencephalography (OEG). In some of any embodiments, the non-invasive measurement of brain activity is EEG.

In some of any embodiments, the non-invasive measurement of brain activity is questionnaire-based evaluation of the experience. In some of any embodiments, the questionnaire-based evaluation of the psychedelic experience is selected from among one or more of: the mystical experience questionnaire (MEQ30) evaluation, the challenging experience questionnaire (CEQ) evaluation, the psychological insight questionnaire (PIQ) evaluation, a qualitative written assessment, and/or the monitor rating scale (MRS) questionnaire.

In some of any embodiments, the methods also involve obtaining a further non-invasive measurement of brain activity from the subject to monitor the dissociative state during the therapeutic window. In some of any embodiments, the methods also involve obtaining a further measurement of EEG from the subject to monitor the dissociative state during the therapeutic window.

In some of any embodiments, the measurement of EEG is analyzed as expressed by Lempel-Ziv complexity (LZC).

In some of any embodiments, the non-invasive measurement of brain activity is obtained prior to, during and/or after administration. In some of any embodiments, the measurement of EEG is obtained prior to, during and after administration.

In some of any embodiments, the non-invasive measurement of brain activity after administration indicates the termination of dissociative state. In some of any embodiments, the measurement of EEG after administration indicates the termination of dissociative state.

In some of any embodiments, the dissociative state is induced within at or about 1, 2, 3, 4, 5, 6, 7, 8, 9, 10, 11, 12, 13, 14, 15, 16, 17, 18, 19, or 20 minutes after the administration. In some of any embodiments, the dissociative state is induced within at or about 5 minutes after the administration.

In some of any embodiments, the therapeutic window is at or about 0.5, 1, 2, 3, 4, 5, or 6 hours. In some of any embodiments, the therapeutic window is between at or about 0.5 hours and at or about 4 hours.

In some of any of the provided embodiments, the methods also involve terminating administration of the psychedelic to terminate the dissociative state.

In some of any embodiments, the termination of dissociative state occurs within at or about 30, 60, 90, 120, or 180 minutes after the termination of administration of the psychedelic, or a range defined by any of the foregoing. In some of any embodiments, the termination of dissociative state occurs within at or about 60 minutes after the termination of administration of the psychedelic.

In some of any embodiments, the psychedelic is administered to produce a predetermined Cₘₐₓ within at or about 30-120 minutes after the initiation of the administration. In some of any embodiments, the psychedelic is administered to produce a predetermined Cₘₐₓ within at or about 45-90 minutes after the initiation of the administration. In some of any embodiments, the psychedelic is administered to produce a predetermined Cₘₐₓ within at or about 1-10 minutes of the administration.

In some of any embodiments, the Cₘₐₓ is at or about 1 μg/L-50 μg/L. In some of any embodiments, the Cₘₐₓ is at or about 1 μg/L-20 μg/L. In some of any embodiments, the Cₘₐₓ is at or about 10 μg/L-20 μg/L. In some of any embodiments, the Cₘₐₓ is at or about 10 μg/L-15 μg/L.

In some of any embodiments, the dissociative state is induced by intravenous administration of a loading dose of the psychedelic.

In some of any embodiments, the loading dose comprises administration of an initial bolus of the psychedelic. In some of any embodiments, the initial bolus is dose is at or about 1 mg, 2 mg, 3 mg, 4 mg or 5 mg.

In some of any embodiments, the loading dose of the psychedelic is administered over a period of at or about 1, 2, 3, 4, 5, 6, 7, 8, 9 or 10 minutes, or a range defined by any of the foregoing. In some of any embodiments, the loading dose of the psychedelic is administered over a period of at or about 3 minutes.

In some of any embodiments, the initial bolus of psychedelic is at a dose of at or about 0.02 mg/kg to at or about 0.2 mg/kg.

In some of any embodiments, the dissociative state is induced by intravenous administration of an initial bolus of the psychedelic at a dose of about 0.1 mg/kg to about 0.2 mg/kg.

In some of any embodiments, the mean plasma concentration of the psychedelic is maintained at the predetermined value during the therapeutic window by administration of a maintenance dose of the psychedelic.

In some of any embodiments, the maintenance dose of the psychedelic is administered by continuous or intermittent administration of the psychedelic. In some of any embodiments, the continuous or intermittent administration is via an intravenous route.

In some of any embodiments, the maintenance dose is administered by an intravenous infusion.

In some of any embodiments, the maintenance dose of the psychedelic is administered by intermittent administration of the psychedelic and the intermittent administration is via a subcutaneous, oral, transdermal, intramuscular, intranasal, intranasal/pharanygeal, or buccal route.

In some of any embodiments, the maintenance dose is administered by intravenous infusion of the psychedelic at a rate of at or about 0.2 mg/min to at or about 1 mg/min.

In some of any embodiments, the continuous administration of the psychedelic is at a rate of at or about 0.1 mg/min to at or about 1 mg/min.

Also provided herein are methods of treating a psychological disorder in a subject. In some of any of the provided embodiments, the methods involve administering to a subject having a psychological disorder a loading dose of a psychedelic by intravenous administration of an initial bolus in an amount between at or about 1 mg and at or about 5 mg; and thereafter continuously administering a maintenance dose of the psychedelic by intravenous infusion at a rate of at or about 0.02 mg/min to at or about 1 mg/min to maintain the mean plasma concentration during a therapeutic window, wherein the psychedelic is psilocybin, psilocin, a co-crystal, a co-former, or a salt thereof, or a combination thereof.

Also provided herein are methods of treating a psychological disorder in a subject. In some of any of the provided embodiments, the methods involve administering to a subject having a psychological disorder a loading dose of a psychedelic by intravenous infusion at a rate of at or about 0.5 mL/min to at or about 2.0 mL/min over a period of at or about 5 minutes to at or about 20 minutes; and thereafter continuously administering a maintenance dose of the psychedelic by intravenous infusion at a rate of at or about 0.1 mL/min to at or about 2.0 mL/min over a period of at or about 30 minutes to at or about 120 minutes, to maintain the mean plasma concentration during a therapeutic window, wherein the psychedelic is psilocybin, psilocin, a co-crystal, a co-former, or a salt thereof, or a combination thereof.

In some of any embodiments, the loading dose of the psychedelic is administered at a rate of at or about 0.1 mL/min to at or about 2.0 mL/min. In some of any embodiments, the loading dose of the psychedelic is administered at a rate of at or about 1.0 mL/min.

In some of any embodiments, the maintenance dose is administered at a rate of 0.1 mL/min to at or about 1.0 mL/min. In some of any embodiments, the maintenance dose is administered at a rate of at or about 0.5 mL/min.

In some of any embodiments, the maintenance dose is administered over a period of at or about 30, 60, 90 or 120 minutes, or a range defined by any of the foregoing. In some of any embodiments, the maintenance dose is administered over a period of up to at or about 60 minutes. In some of any embodiments, the maintenance dose is administered over a period of up to at or about 120 minutes. In some of any embodiments, the therapeutic window is between at or about 30 minutes to at or about 120 minutes.

In some of any embodiments, the therapeutic window is at or about 60 minutes. In some of any embodiments, the therapeutic window is at or about 120 minutes.

In some of any embodiments, the total amount of psychedelic that is administered to the subject is up to at or about 1.0 mg, 1.5 mg, 2.5 mg, 5 mg. 7.5 mg, 10 mg, 15 mg or 20 mg per subject, or a range defined by any of the foregoing. In some of any embodiments, the total amount of psychedelic that is administered to the subject is up to at or about 2.5 mg. In some of any embodiments, the total amount of psychedelic that is administered to the subject is up to at or about 5 mg. In some of any embodiments, the total amount of psychedelic that is administered to the subject is up to at or about 10 mg.

In some of any embodiments, the psychedelic is psilocin, a co-crystal, co-former, or salt thereof. In some of any embodiments, the psychedelic is psilocin.

In some of any embodiments, the psychedelic is psilocybin, a co-crystal, co-former, or salt thereof. In some of any embodiments, the psychedelic is psilocybin.

In some of any embodiments, the psychedelic is psilocin, and the total amount of psilocin that is administered to the subject is up to at or about 2.5 mg, and the administration of psilocin is carried out over a period of at or about 60 minutes.

In some of any embodiments, the psychedelic is psilocybin, and the total amount of psilocybin that is administered to the subject is up to at or about 5 mg, and the administration of psilocybin is carried out over a period of at or about 60 minutes.

In some of any embodiments, the predetermined value of mean plasma concentration of the psychedelic is at or about 1-50 μg/L. In some of any embodiments, the predetermined value of mean plasma concentration of the psychedelic is at or about 10-20 μg/L. In some of any embodiments, the predetermined value of mean plasma concentration of the psychedelic is at or about 10-15 μg/L.

In some of any embodiments, the methods also involve providing psychological support to the subject during the therapeutic window.

In some of any embodiments, the methods also involve withdrawing the subject from the dissociative state at the end of the treatment window by administering a 5HT_{2A} receptor antagonist.

In some of any embodiments, the psychological disorder is selected from the group consisting of PTSD, alcohol addition, drug addiction, treatment resistant depression, anxiety, end of life anxiety, an eating disorder, fibromyalgia, neuropathic pain, phantom limb pain, hypothalamic induced obesity, Prader-Willi syndrome, and binge-eating disorder. In some of any embodiments, the psychological disorder is selected from the group consisting of hypothalamic induced obesity, Prader-Willi syndrome, binge-eating disorder, and fibromyalgia.

In some of any embodiments, the psychological disorder is a nociplastic pain disorder. In some of any embodiments, the psychological disorder is fibromyalgia. In some of any embodiments, the psychological disorder is phantom limb pain. In some of any embodiments, the psychological disorder is complex regional pain syndrome.

In some of any embodiments, the methods also involve discontinuing the administration if the subject experiences an adverse event.

In some of any embodiments, the psychedelic is administered with at least one additional therapeutic agent.

In some of any embodiments, the method results in the induction of dissociative state in at least 70%, 80%, 90% or 95% of a plurality of subjects that have been administered the psychedelic.

In some of any embodiments, the method results the maintenance of the mean plasma level of the psychedelic in at least 70%, 80%, 90% or 95% of a plurality of subjects that have been administered the psychedelic.

In a first aspect, there is provided a method of treating a psychological disorder in a subject comprising administering to the subject an amount of a psychedelic sufficient to induce a dissociative state in the subject less than 30 minutes after administration; and thereafter maintaining the mean plasma concentration of the psychedelic at a predetermined value to maintain the dissociative state during a therapeutic window.

In one embodiment, the dissociative state is induced within 1, 2, 3, 4, 5, 6, 7, 8, 9, about 10, about 15, about 20, about 25, or less than about 30 minutes. The therapeutic window may be about 0.5, 1, 2, 3, 4, 5, or 6 hours.

In one embodiment, the Cₘₐₓ of the psychedelic occurs within 1 to 10 minutes of the administration. The Cₘₐₓ may be from 1 to at least about 50 g/L.

In some embodiments, the dissociative state is induced by administration of an initial intravenously administered bolus of the psychedelic at a dose of about 0.01 to 0.2 mg/kg.

In some embodiments, the dissociative state is induced by administration of an initial intravenously administered bolus of psilocybin or psilocin. The initial intravenous dose of psilocybin, psilocin or a combination thereof may be about 1, 2, 3, 4, or 5 mg. Alternatively, the initial intravenous dose of psilocybin may be about 0.01-0.2 mg/kg, for example 0.01, 0.02, 0.03, 0.04, 0.05, 0.06, 0.07, 0.08, 0.09, or about 0.1 mg/kg.

In some embodiments, the dissociative state is induced by administration of an initial oral dose of psilocybin, psilocin or a combination thereof may be 0.1-1.0 mg/kg, for example, 0.1, 0.2, 0.3, 0.4, 0.5, 0.6, 0.7, 0.8, 0.9, or 1.0 mg/kg. Alternatively, the initial oral dose of psilocybin, psilocin or a combination thereof may be 10-40 mg, for example 10, 15, 20, 25, 30, 35 or 40 mg.

After the initial dose the mean plasma concentration of the psychedelic is maintained at the predetermined value during the therapeutic window by continuous or intermittent administration of the psychedelic. For example, the psychedelic may be continually administered intravenously at a rate of 0.02 mg/min to 1 mg/min. In some embodiments the psychedelic may be continuously administered transdermally. Alternatively the psychedelic may be intermittently administered for example intravenously, or orally.

In an embodiment, the predetermined plasma concentration of the psychedelic is from about 1 μg/L-50 μg/L.

In one embodiment the psychedelic is psilocybin, psilocin or combination thereof and is administered intravenously to maintain the mean plasma concentration of the psychedelic at the predetermined value.

The intravenous dose of psilocybin, psilocin or combination thereof may be about 1, 2, 3, 4, or 5 mg. Alternatively, the intravenous dose of psilocybin may be about 0.01-0.2 mg/kg, for example, 0.01, 0.02, 0.03, 0.04, 0.05, 0.06, 0.07, 0.08, 0.09, or about 0.1 mg/kg. 0.11, 0.12, 0.13, 0.14, 0.15, 0.16, 0.17, 0.18, 0.19, or 0.2 mg/kg.

In one embodiment the psilocybin, psilocin or combination thereof is administered orally.

The oral dose of psilocybin, psilocin or combination thereof may be about 2 mg to at least about 15 mg, for example, 2, 3, 4, 5, 6, 7, 8, 9, 10, 11, 12, 13, 14, or at least about 15 mg. Alternatively, the oral dose of psilocybin may be about 0.05-0.2 mg/kg, for example 0.05, 0.06, 0.07, 0.08, 0.09, 0.1, 0.11, 0.12, 0.13, 0.14, 0.15, 0.16, 0.17, 0.18, 0.19, or 0.2 mg/kg.

The method further comprises providing psychological support to the subject during the therapeutic window.

The method may further comprise withdrawing the subject from the dissociative state at the end of the treatment window or if the patient is experiencing adverse events by administering a 5HT_{2A} receptor antagonist, such as risperidone, olanzapine, mirtazapine or mianserin.

The psychological disorder may be selected from the group consisting of PTSD, alcohol addition, drug addiction, treatment resistant depression, anxiety, end of life anxiety, an eating disorder, fibromyalgia, neuropathic pain, phantom limb pain, hypothalamic induced obesity, and Prader-Willi syndrome, binge eating disorders. Preferably, the psychological disorder is hypothalamic-induced obesity, Prader-Willi syndrome, binge eating or fibromyalgia.

In some embodiments, the psychedelic is psilocybin, psilocin or a combination thereof.

In some embodiments, the psychedelic is administered with at least one additional therapeutic agent for the psychological disorder.

In a preferred embodiment, the psychedelic is administered intravenously.

In some embodiments, the psychedelic is administered with another pharmaceutical agent either together or sequentially.

In a second aspect there is provided a method of determining a therapeutically effective dose of a psychedelic for treating a psychological disorder in a subject, the method comprising;
administering to the subject an amount of psilocybin or psilocin sufficient to induce a dissociative state in the subject less than 30 minutes after administration; and
obtaining an EEG from the subject to determine when the subject enters the dissociative state;
thereafter maintaining the mean plasma concentration of the psychedelic at a predetermined value to maintain the dissociative state during a therapeutic window.

In some embodiments the method may further comprising obtaining a further EEG from the subject to monitor the dissociative state during the therapeutic window.

In some of any embodiments, provided herein are uses of a psychedelic that is psilocybin, psilocin, a co-crystal, a co-former, or a salt thereof, or a combination thereof, in accordance with any of the embodiments described herein, including the methods of treating a psychological disorder, methods of inducing a dissociative state, methods of determining a therapeutically effective dose of a psychedelic, and methods of maintaining a dissociative state in a subject with a psychological disorder, methods of treating phantom limb pain in a subject. In some of any embodiments, provided herein is a psychedelic that is psilocybin, psilocin, a co-crystal, a co-former, or a salt thereof, or a combination thereof, for use in any of the embodiments described herein, including the methods of treating a psychological disorder, methods of inducing a dissociative state, methods of determining a therapeutically effective dose of a psychedelic, and methods of maintaining a dissociative state in a subject with a psychological disorder, methods of treating phantom limb pain in a subject. In some of any embodiments, provided herein are uses of a psychedelic that is psilocybin, psilocin, a co-crystal, a co-former, or a salt thereof, or a combination thereof, in the manufacture of a medicament for use in accordance with any of the embodiments described herein, including the methods of treating a psychological disorder, methods of inducing a dissociative state, methods of determining a therapeutically effective dose of a psychedelic, and methods of maintaining a dissociative state in a subject with a psychological disorder, methods of treating phantom limb pain in a subject.

### BRIEF DESCRIPTION OF THE DRAWINGS

FIG. 1A and FIG. 1B show the concentration of plasma psilocin of three rats during and after a one-hour intravenous infusion of 10 mg/kg psilocin (FIG. 1A) or psilocybin (FIG. 1B).

FIG. 2A and FIG. 2B show the normalized temporal Lempel-Ziv complexity (LZC_{N}) estimations as determined based on electroencephalography (EEG) in three rats before, during, and after a one-hour intravenous infusion of 10 mg/kg psilocybin. FIG. 2A shows individual temporal LZC_{N} values. FIG. 2B shows a box plot distribution of temporal LZC_{N} values.

FIG. 3 shows the concentration of plasma psilocin in rats after a 3-minute intravenous infusion of 10 mg/kg psilocybin, with rats having either received (ISO+) or not received (ISO−) isoflurane anesthetic prior to psilocybin infusion.

FIG. 4A and FIG. 4B show Von Frey assay (VFA) withdrawal threshold in a formalin-induced rat pain model (rats injected with formalin in a hind paw) and either 10 mg/kg psilocybin or saline via intravenous infusion. FIG. 4A shows withdrawal threshold for the injected paw. FIG. 4B shows withdrawal threshold for the contra-lateral paw.

FIG. 5 shows Hot Plate assay (HPA) withdrawal latency from a 52.5° C. hot plate in a formalin-induced rat pain model (rats injected with formalin in a hind paw) and either 10 mg/kg psilocybin or saline via intravenous infusion.

FIG. 6 shows an exemplary projected concentration of plasma psilocin in human subjects over 360 minutes after administration of psilocin or psilocybin, either orally (PO) or by intravenous infusion. The plot includes observed plasma concentration as described in Brown et al. (2017). Clin Pharmacokinet. 56(12):1543-1554.

### DETAILED DESCRIPTION

Provided herein are methods of inducing a dissociative state or psychedelic state and methods of treatment that involve the administration, for example by intravenous administration, of psychedelics, such as psilocin or psilocybin. In some aspects, also provided are methods of determining a therapeutically effective dose of a psychedelic, for example, for a method of treatment or a treatment regimen. Provided herein are methods for treating or ameliorating a psychological disorder, for example, a disease having a psychological component and certain pain related disorders, such as nociplastic pain disorders. In some aspects, the provided methods involve administering psychedelics, such as psilocin or psilocybin, for example, via intravenous administration. In some aspects, provided herein are methods of administering and uses, such as therapeutic and prophylactic uses, of psychedelics for treating or ameliorating psychological disorders or one or more symptoms thereof. In some aspects, also provided are methods of that involve the monitoring of the dissociative state or psychedelic state of the subject before, during or after administration of a psychedelic. In some aspects, the provided methods involve monitoring by one or more non-invasive measurements of brain activity, for example, electroencephalography (EEG). In some aspects, also provided are methods for determining a therapeutically effective dose of a psychedelic, for example, for a method of treatment or a treatment regimen. In some aspects, the provided methods and uses leads to improvement, such as clinical improvement, or amelioration of the psychological disorder.

In some embodiments, psychedelics (e.g., psilocin or psilocybin) and compositions comprising psychedelics are useful in a variety of therapeutic, diagnostic, and prophylactic indications. For example, the compositions are useful in treating a variety of diseases and disorders such as psychological disorders in a subject. Such methods and uses include therapeutic methods and uses, for example involving administration of the psychedelic to a subject having a disease, condition, or disorder, such as a psychological disorder or disease. In some embodiments, the psychedelics (e.g., psilocin or psilocybin) are administered in an effective amount to effect treatment of the disease or disorder. Uses include uses of the compositions in such methods and treatments, and in the preparation of a medicament in order to carry out such therapeutic methods. In some embodiments, the methods are carried out by administering the psychedelics (e.g., psilocin or psilocybin) to the subject having or suspected of having the disease or condition, such as a psychological disorder. In some embodiments, the methods thereby treat the disease, condition, or disorder in the subject.

Also provided herein are uses of psychedelics, such as psilocin or psilocybin, in such methods and treatments, and in the preparation of a medicament in order to carry out such methods. In some embodiments, the methods and uses thereby improve, ameliorate and/or treat the psychological disorder or one or more symptoms thereof, in the subject. In some embodiments, the provided therapeutic methods and uses that involve the administration, such as intravenous administration, of psychedelics, such as psilocin or psilocybin, a subject having, exhibiting or suffering from one or more symptoms of a psychological disorder, in an amount that is sufficient to rapidly induce a dissociative state or a psychedelic state in the subject, and/or an effective amount to improve or ameliorate one or more symptoms of the psychological disorder.

Psilocybin (4-phosphoroyloxy-N,N-dimethyltryptamine) is a tryptamine psychedelic that has similar effects to dimethyltryptamine (DMT), lysergic acid diethylamide (LSD) and mescaline. These agents produce psychoactive effects that have been characterized as forming an intense dream-like state with colorful visual illusions, changes in auditory, tactile, olfactory, gustatory, and kinesthetic perceptions, altered perceptions of time and space, changes in body image, and sensations including ego dissolution, and intense mood changes ranging from feelings of wonder and bliss to sadness and grief.

Psilocybin administered in an appropriately controlled and supportive setting can have long lasting positive psychological effects and long-term positive effects have been observed in clinical studies showing a decrease in depressive and anxiety symptoms and abstinence from addictive substances.

A common mechanism of action of psychedelics (including psilocybin) is via serotonergic (5-HT) pathways. Psilocybin is a prodrug of psilocin which is a strong 5-HT_{2A} receptor agonist as well as a moderate agonist of 5-HT_{1A} and 5-HT_{2C} receptors. Activation of 5-HT_{2A} receptors in the thalamus (the area of the brain that relays sensory information) decreases thalamic activity and leads to sensory alterations commonly referred to as hallucinations. The direct 5-HT_{2A} receptor agonist properties of psilocybin enhances sensitivity to the environment as well as facilitating emotional release, which, when combined with psychological support, is hypothesized to be therapeutically potent. That is, due to the ability of psilocybin to alter sensory perception and serotonergic activity it has been investigated as a treatment for mental health conditions characterized by abnormalities in sensory perception, such as major depressive disorder, treatment-resistant depression, anxiety or anxiety-related disorders. Psilocybin has also been researched for use in substance abuse disorders.

Psilocybin is a prodrug and is converted to the active psilocin in vivo by the action of alkaline phosphatase. Psilocybin's threshold for intoxication is approximately 0.20 mg/kg of body weight and after oral administration hallucinogenic effects typically onset within the first 60 to 90 minutes of use then cease within 3 to 6 hours although there is considerable inter-subject variation which presents a problem for conventional use of psychedelic substances to treat psychological disorders. Specifically, after administration of a psychedelic such as psilocybin there is wide inter-subject variation in area under the plasma concentration versus time curve, a great deal of inter-subject variation in Cₘₐₓ and Tₘₐₓ and hence the time to induction of a dissociative state and the duration of the dissociative state (which is required for effective treatment). That is, because of the wide inter-subject variability in the pharmacokinetic parameters of psilocybin and other psychedelics) it is difficult to predict or control the therapeutic window for psilocybin, psilocin, and other psychedelics. Accordingly, typical doses of psilocybin, particularly when administered orally, can result in patients achieving plasma concentrations that are either too high which can lead to psychosis (a ‘bad trip’) or too low and such that a dissociative state is not achieved.

Another problem with treatment using psychedelic substances such as psilocybin and LSD is that the subject needs to be with 2 therapists for a minimum of 8-10 hours. The therapists need to be with the patient prior to administration of the psychedelic and a lag time of up to 3 hours can occur prior to the patient entering the dissociative state, which can last for up to an additional 6 hours, and which is well beyond a useful therapeutic window.

The present inventors have developed formulations and methods that allow dosing control for psilocin, psilocybin and other psychedelics to enable more effective therapeutic use of psychedelics. Provided are compositions, methods and uses that can address the difficulties and limitations of existing methods of administering psychedelics.

The provided embodiments, including the methods, allow a treating physician to control one or more of the following parameters:
Time to onset of dissociative state
Duration of dissociative state
Cₘₐₓ of the psychedelic drug.

The provided embodiments, including the methods, also allow a treating physician to monitor and control one or more of the following parameters
Time to entrance into the dissociative state
Exit from the dissociative state
Mean plasma concentration of the psychedelic
Duration of administration
Termination of administration if adverse events occur.

Control of these parameters offers a number of advantages including avoidance of high plasma concentrations of the psychedelic, which may lead to the subject experiencing a psychotic episode, or placing the subject at high risk of experiencing adverse events, such as a psychotic episode. In some aspects, high plasma concentration is associated with adverse events or side effects that are cardiovascular in nature, such as an increases in blood pressure, heart rate and the risk of tachycardia or arrhythmia, and the provided embodiment that permit the control of the plasma levels and avoidance of high plasma concentrations, improves therapeutic interventions with psychedelics while minimizing adverse effects. Additionally, controlling these parameters can be used to avoid unwanted effects of the psychedelic such as one or more of increased blood pressure, tachycardia, panic attacks, hallucinogen persisting perceptual disorder (HPDD), unpleasant emotional reactions (a ‘bad trip’). Accordingly, the embodiments, including the methods, disclosed herein provide various advantages, such as the ability to monitoring the dissociative state, determining therapeutically effective doses, and assessing, adjusting and tailoring the therapeutic treatment depending on the subject's response.

In some aspects, the provided embodiments are based on observations described herein, that intravenous administration of a psychedelic, such as psilocin or psilocybin, results in reproducible and consistent pharmacokinetic and pharmacodynamics profiles in different subjects. In some aspects, as described herein, intravenous administration of the psychedelic, such as by an intravenous infusion, results in a rapid and consistent plasma level of the psychedelic, accompanied by entrance into a dissociative state, measured using a non-invasive measurement of brain activity, such as EEG. In some aspects, upon termination of the administration, the rapid decrease in plasma levels and the concomitant exit from the dissociative state, are also observed. The observations also support the utility of various exemplary non-invasive measurement of brain activity, such as EEG or observing behavior, to monitor the subject's dissociative state, and permit control and change the dose of the psychedelic, and the pharmacokinetic parameters, such as plasma concentration and/or exposure, of the psychedelic.

In some aspects, the provided embodiments are also based on an observation that intravenous administration of a psychedelic exhibited beneficial effects on pain perception in an animal model of pain that is relevant for diseases and indications associated with pain, such as nociplastic pain. In some aspects, the demonstrated effect of intravenous psychedelic administration on pain perception in the non-injected paw that is prolonged for weeks after administration, supports the utility of the provided embodiments in the treatment of pain related disorders, such as nociplastic pain disorders, for example, fibromyalgia, phantom limb pain or complex regional pain syndrome. In some aspects, the observations support the advantages of the provided embodiments.

In some aspects, the treatment also involves psychotherapy. In some aspects, the provided methods and uses also involve psychotherapy, for example, in one or more clinical sessions. In some embodiments, the provided therapeutic methods and uses, for example, involving administration of psychedelics, in some cases in combination with psychotherapy, to a subject.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Methods of Treatment and Uses Involving Administration of Psychedelic Compounds

Provided herein are methods and uses that involve administration of psychedelics such as psilocin or psilocybin. In some aspects, the provided embodiments involve intravenous administration of the psychedelic. In some aspects, the provided embodiments involve intravenous administration of one or more doses of the psychedelic, such as one or more doses of psilocin or one or more doses of psilocybin. In some aspects, the provided embodiments involve intravenous administration over a period of time, for example, an intravenous infusion of the psychedelic. In some aspects, the provided embodiments involve administering a psychedelic in an amount sufficient to induce a dissociative state in the subject. In some embodiments, the individual also receives psychotherapy, for example, in one or more clinical session, in combination or in conjunction with administration of the psychedelic.

The methods disclosed herein involve the administration of a psychedelic such as psilocin or psilocybin to a subject to treat or prevent a psychological disorder. Typically, the methods involve intravenous administration of the psychedelic to avoid the hepatic first pass metabolism. Other routes of administration that avoid the hepatic first pass effect, such as intranasal, intranasal/pharyngeal, and buccal routes, transdermal are also contemplated. Parenteral administration via the subcutaneous and intra-muscular route are also contemplated. The methods also require the provision of psychological support to the subject by a therapist, psychiatrist, psychotherapist or other suitably qualified health professional.

The methods require the subject to enter a ‘psychedelic state’ or ‘dissociative state,’ which is characterized by effects that are placed into three categories:
Perceptual effects: altered shapes and colors (often appearing brighter, more vivid, and intense); visual distortions; visual hallucinations (open eye visuals and closed eye visuals); illusions; difficulty focusing; synaesthesias.
Psychic effects: mood alterations (from ecstatic euphoria to panic); time distortion; thought alterations (difficulty concentrating, strange concepts, ideas, or connections, increases in creativity); dreamlike feelings; depersonalization; the sense that awareness is ‘expanded’.
Somatic effects: dizziness; tremors; nausea; drowsiness; blurred vision.

The relative prominence of particular effects can be strongly dependent on the specific drug, the dosage, and the setting in which the drug is used. However, with psilocybin and psilocin, the dissociative state is characterized by an unconstrained form of cognition in which the stream of conscious experience appears more fluid and dynamic, with novel neural states being explored by the subject.

### A. Dosing

In some aspects, provided are methods of treatment and uses that involve administering to a subject a psychedelic, such as psilocin or psilocybin, in an amount of a psychedelic sufficient to induce a dissociative state in the subject. In some aspects, the amount of the psychedelic is sufficient to induce a dissociative state in the subject within less than at or about 15, 30, 60, 90 or 120 minutes after administration, or a range defined by any of the foregoing. In some aspects, the amount of the psychedelic is sufficient to induce a dissociative state in the subject within less than at or about 30 minutes after administration (for example, less than at or about 30 minutes after the initiation of administration of the psychedelic, or less than at or about 30 minutes after the termination of administration of the psychedelic). In some aspects, the embodiments also involve maintaining the mean plasma concentration of the psychedelic at a predetermined value to maintain the dissociative state during a therapeutic window.

In some aspects, the psychedelic is administered by intravenous administration. In some aspects, the psychedelic is administered by an initial intravenous bolus injection. In some embodiments, the psychedelic is administered by an intravenous infusion, e.g., continuous infusion and/or delivering a steady rate of the psychedelic. In some aspects, both an initial bolus injection and an intravenous infusion is administered.

In some aspects, the provided methods involve administration, such as by intravenous administration, of an amount of a psychedelic sufficient to induce a dissociative state in the subject. In some aspects, the amount of a psychedelic that is psilocybin or psilocin sufficient to induce a dissociative state in the subject less than at or about 15, 30, 60, 90 or 120 minutes after the administration, or a range defined by any of the foregoing, such as less than at or about 30 minutes after administration.

In some aspects, the provided embodiments also involve maintaining the mean plasma concentration of the psychedelic at a predetermined value to maintain the dissociative state during a therapeutic window. In some aspects, the pharmacokinetic parameter of the administered psychedelic or a metabolite thereof (e.g., psilocin), is assessed and/or monitored in the subject. In some aspects, the provided embodiments achieve a plasma concentration, such as a mean plasma concentration, of the psychedelic, at a predetermined value. In some aspects, the predetermined value is sufficient to maintain the psychedelic state during a therapeutic window. In some aspects, the amount or dose of the psychedelic is determined as a therapeutically effective dose if the subject enters the dissociative state.

In some aspects, the pharmacokinetic parameter is the plasma level of the psychedelic or metabolite thereof. In some aspects, the plasma concentration of psilocin is measured. In some embodiments, the maximum plasma concentration (Cₘₐₓ), time to maximum plasma concentration (Tₘₐₓ), and/or the area under the curve (AUC) is determined as a pharmacokinetic parameter. In some embodiments, the dosage, rate or timing of the administration is tailored, customized, changed or adjusted based on the determined pharmacokinetic parameters.

Exemplary pharmacokinetic (PK) parameters used to assess a pharmacological drug response and bioavailability include Cₘₐₓ, Tₘₐₓ, and AUC (Bialer et al, J Pharm Sci 1995; 84(10): 1160-3). These parameters are can be used to determine the extent and rate of absorption of immediate-release drug products. A PK analysis can involve blood sampling from which plasma is obtained and subsequently analyzed for drug metabolite levels (e.g., the psychedelic or metabolite thereof, such as psilocin). In some aspects, the pharmacokinetic parameter analysis provides the plasma concentration of the metabolite (e.g., psilocin) at a given time during the treatment window.

Cₘₐₓ is defined as the highest concentration of a drug in the plasma, or other biological material use for the analysis (e.g., blood, cerebrospinal fluid, or target organ) after a dose is administered. As the drug is processed (i.e., absorption, distribution, metabolism, and excretion), the plasma concentration of the subject fluctuates, and time it takes for a drug to reach the maximum concentration (Cₘₐₓ) after administration of a drug is the Tₘₐₓ. Tₘₐₓ is governed by the rates of drug absorption and drug elimination.

The area under the plot of plasma concentration of a drug versus time after dosage (i.e., area under the curve or AUC), which can be expressed in units of mg*h/L, is associated with the extent of drug exposure and clearance from the body (Scheff et al., Pharm Res 2011; 28(5): 1081-9). Integrating over time yields a more accurate estimate of the overall drug exposure compared to than relying on individual concentration measurements. Further, these measurements are useful for assessing the net pharmacologic response to a given dose of drug (Krzyzanski and Jusko, J Pharm Sci 1998; 87(1):67-72).

For AUC, this parameter can be calculated as AUC (0-t) or AUC (0-∞). In general, AUC (0-t) is defined as the area under the concentration-time curve from dosing at time=0 to time=t. AUC (0-t) can be computed for multiple values of t=time to gain a better understanding of the PK drug profile. In some cases, AUC (0-∞) is the AUC curve extrapolated to infinite time, as the duration of an assay is always finite. AUC (0-∞) is calculated from AUC (0-t) with the addition of a constant (Clast/λz), where Clast is the last observed quantifiable concentration and λz is the terminal phase rate constant. AUC values are often calculated computationally due to the complexities and large number of variables involved in tracking drug metabolism.

In some embodiments, the pharmacokinetic parameter, such as plasma concentration, Cₘₐₓ or AUC, is indicative of or associated with the entry into and/or exit from the dissociative state. In some aspects, the pharmacokinetic parameter is indicative of or associated with one or more non-invasive measurements of brain activity, such as EEG.

Peak plasma levels of psilocin after a single oral administration of psilocybin at 0.225 mg/kg occur after 105+/−37 min showing an average concentration of 8.2+/−2.8 ng psilocin/ml plasma and estimates for the absolute bioavailability of psilocin after oral administration of psilocybin are 52.7+/−20% (Hasler et al, Pharm Acta Helv. 1997 June; 72(3):175-84).

In contrast, in some embodiments the methods disclosed herein utilize psilocin or psilocybin doses to achieve a plasma psilocin concentration 1-20 μg/L to induce a dissociative state.

In some embodiments, the psychedelic is administered to produce a predetermined Cₘₐₓ of between at or about 1 μg/L and at or about 50 μg/L, such as at or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 μg/L, or a range defined by any of the foregoing. In some aspects, the predetermined Cₘₐₓ is from 1-20 μg/L. For example the predetermined Cₘₐₓ can be about 1 μg/L. 2 μg/L, 3 μg/L, 4 μg/L, 5 μg/L, 6 μg/L, 7 g/L, 8 μg/L. 9 μg/L. 10 μg/L, 11 μg/L, 12 μg/L, 13 μg/L, 14 μg/L, 15 μg/L, 16 μg/L, 17 μg/L, 18 μg/L, 19 μg/L, or about 20 μg/L.

In some embodiments, the psychedelic is administered to produce a predetermined Cₘₐₓ occurs within at or about 1 minute to at or about 120 minutes after the initiation of the administration, such as within at or about 1, 2, 3, 4, 5, 6, 7, 8, 9, 10, 15, 20, 30, 40, 50, 60, 70, 80, 90, 100, 110 or 120 minutes after the initiation of administration, or a range defined by any of the foregoing. In some aspects, the psychedelic is administered to produce a predetermined Cₘₐₓ within at or about 30-120 minutes after the initiation of the administration. In some aspects, the psychedelic is administered to produce a predetermined Cₘₐₓ within at or about 45-90 minutes after the initiation of the administration, such as at or about 60 minutes after the initiation of administration. In some aspects, the psychedelic is administered to produce a predetermined Cₘₐₓ within at or about 1-10 minutes after the initiation of the administration. In some embodiments, the psychedelic is administered to produce a predetermined Cₘₐₓ occurs within 1-10 minutes of administration. In some aspects, the psychedelic is administered to produce a predetermined Cₘₐₓ within at or about 1-10 minutes after the initiation of the administration.

In some embodiments, the dissociative state can be induced and a suitable therapeutic window generated with a single dose of about 0.01 mg/kg, about 0.02 mg/kg to about 0.5 mg/kg of the psychedelic. Suitable single doses include about 0.02 mg/kg, about 0.05 mg/kg, about 0.1 mg/kg, about 0.15 mg/kg, about 0.2 mg/kg, about 0.25 mg/kg, about 0.3 mg/kg, about 0.35 mg/kg, about 0.4 mg/kg, about 0.45 mg/kg, or about 0.5 mg/kg.

The induction of the dissociative state in the subject marks the start of the therapeutic window, which is associated with the range of plasma concentrations of the psychedelic compound at which treatment will be effective without having negative effects (such as a psychotic episode or tachycardia). In some aspects, the therapeutic window is a period of time in which the mean plasma concentration of the psychedelic is maintained at a predetermined value to maintain the dissociative state. In some aspects, the therapeutic window is between at or about 15 minutes to at or about 12 hours, such as at or about 0.5, 1, 2, 3, 4, 5, or 6 hours, or a range defined by any of the foregoing. In some embodiments, the therapeutic window is between at or about 0.5 and at or about 4 hours, such as at or about 1 hour or at or about 2 hours.

In some of any of the provided embodiments, the dissociative state is induced by administration of a loading dose (for example, an initial dose) of the psychedelic. In some aspects, the loading dose of the psychedelic is administered by intravenous administration. In some aspects, the loading dose comprises administration of an initial bolus injection of the psychedelic. In some aspects, the loading dose comprises continuous intravenous administration, such as an intravenous infusion. In some aspects, the loading dose is administered by an intravenous infusion, at an initial rate of infusion followed by a slower rate to maintain the blood levels. In some aspects, the loading dose of the psychedelic is administered over a period of at or about 1, 2, 3, 4, 5, 6, 7, 8, 9 or 10 minutes, or a range defined by any of the foregoing. In some aspects, the loading dose of the psychedelic is administered over a period of at or about 3 minutes.

In some aspects, exemplary loading dose of the psychedelic, such as the psilocybin, psilocin or a combination thereof, is at or about 0.1 mg to at or about 10 mg, such as at or about 1, 2, 3, 4, or 5 mg, or a range defined by any of the foregoing. In some aspects, the loading dose is an the initial bolus of psychedelic, and the initial bolus is administered at a dose of at or about 0.02 mg/kg to at or about 0.2 mg/kg, such as at or about 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, or 0.2 mg/kg, or a range defined by any of the foregoing.

In some embodiments, the dissociative state is induced by administration of an initial intravenous bolus injection of the psychedelic at a dose of at or about 0.01 mg/kg to at or about 0.2 mg/kg, or at or about 0.02 mg/kg to at or about 0.2 mg/kg, such as at or about 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, or 0.2 mg/kg, or a range defined by any of the foregoing. In some aspects, the dissociative state is induced by intravenous administration of an initial bolus of the psychedelic at a dose of about 0.1 mg/kg to about 0.2 mg/kg. In some embodiments, the initial intravenous dose of psilocybin, psilocin or a combination thereof is at or about 5 mg/kg to at or about 15 mg/kg.

In some aspects, the loading dose of the psychedelic is administered by a continuous administration, such as an intravenous infusion, at an initial rate. In some aspects, exemplary initial rate of infusion is at or about 0.1 mL/min to at or about 2.0 mL/min, such as at or about 0.1, 0.25, 0.5, 0.75, 1.0, 1.25, 1.5, 1.75 or 2.0 mL/min, or a range defined by any of the foregoing. In some aspects, the loading dose of the psychedelic is administered at a rate of at or about 1.0 mL/min.

In some embodiments, the provided methods and uses leads to achieving the dissociative or psychedelic state in the subjects, more rapidly compared to other methods (such as by oral administration). In some aspects, the dissociative state is induced within at or about 30 minutes of administration of the psychedelic, such as within at or about 30 minutes of the initiation of administration of the psychedelic. In some aspects, the dissociative state is induced within at or about 1 to at or about 30 minutes of administration of the psychedelic, such as at or about 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 minutes, or a range defined by any of the foregoing, of the administration of the psychedelic, such as the initiation of administration of the psychedelic. In some aspects, the dissociative state is induced within at or about 1, 2, 3, 4, 5, 6, 7, 8, 9, 10, 11, 12, 13, 14, 15, 16, 17, 18, 19, or 20 minutes of administration. In some embodiments, the dissociative state is induced within at or about 5 minutes of administration.

In other embodiments, after the Cₘₐₓ is reached and the dissociative state is induced, the rate of administration of the psychedelic can be maintained or altered to maintain the therapeutic window. For example, in embodiments where the psychedelic is administered by intravenous infusion, the psychedelic may be continually infused into the subject, or the infusion rate can be reduced to maintain a plasma concentration of the psychedelic just high enough to maintain the dissociative state. Suitable infusion rates will depend on the concentration of the psychedelic in the infusate and can be determined by the skilled person to for example maintain a predetermined plasma concentration of the psychedelic (i.e., predetermined value of mean plasma concentration of the psychedelic), which may be from at or about 1 μg/L to at or about 50 μg/L, such as at or about 1 μg/L to at or about 20 μg/L, for example at or about 1 μg/L, 2 μg/L. 3 μg/L. 4 μg/L. 5 μg/L, 6 μg/L, 7 g/L, 8 g/L, 9 μg/L, 10 μg/L, 11 μg/L, 12 μg/L, 13 μg/L, 14 μg/L, 15 μg/L, 16 μg/L, 17 μg/L, 18 μg/L, 19 μg/L, or 20 μg/L. In some embodiments, the predetermined value of mean plasma concentration of the psychedelic is between at or about 1 μg/L and at or about 50 μg/L, such as at or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 μg/L, or a range defined by any of the foregoing. In some aspects, the predetermined value of mean plasma concentration of the psychedelic is at or about 10-20 μg/L, such as at or about 10-20 μg/L, or at or about 10-15 μg/L.

In some aspects, the embodiments involve maintaining the dissociative state in the subject, for example by maintaining the mean plasma concentration of the psychedelic at a predetermined value to maintain the dissociative state during a therapeutic window. In some aspects, the mean plasma concentration of the psychedelic is maintained at the predetermined value during the therapeutic window by administration of a maintenance dose of the psychedelic.

In some aspects, the maintenance dose maintenance dose of the psychedelic is administered by intravenous or continuous infusion, or intermittent administration of the psychedelic. In some aspects, the maintenance dose is administered by an intravenous infusion. In some aspects, the continuous or intermittent administration is via an intravenous route. In some aspects, the maintenance dose is administered by intermittent administration. In some aspects, the intermittent administration is via a subcutaneous, oral, transdermal, intramuscular, intranasal, intranasal/pharanygeal, or buccal route.

In some embodiments, the maintenance dose is administered at a rate of at or about 0.1 mL/min to at or about 1.0 mL/min, such as at or about 0.1, 0.2, 0.3, 0.4, 0.5, 0.6, 0.7, 0.8, 0.9, or 1.0 mL/min, or a range defined by any of the foregoing. In some embodiments, the maintenance dose is administered by intravenous infusion of the psychedelic at a rate of at or about 0.1 mL/min to at or about 1.0 mL/min, such as at or about 0.1, 0.2, 0.3, 0.4, 0.5, 0.6, 0.7, 0.8, 0.9, or 1.0 mL/min, or a range defined by any of the foregoing. In some aspects, the maintenance dose is administered by intravenous infusion at a rate of at or about 0.5 mL/min.

In some embodiments, the maintenance dose is administered at a rate of at or about 0.2 mg/min to at or about 1.0 mg/min, such as at or about 0.2, 0.3, 0.4, 0.5, 0.6, 0.7, 0.8, 0.9, or 1.0 mg/min, or a range defined by any of the foregoing. In some aspects, the maintenance dose is administered by intravenous infusion of the psychedelic at a rate of at or about 0.2 mg/min to at or about 1.0 mg/min, such as at or about 0.2, 0.3, 0.4, 0.5, 0.6, 0.7, 0.8, 0.9, or 1.0 mg/min, or a range defined by any of the foregoing. In some aspects, the continuous administration of the psychedelic is at a rate of at or about 0.1 mg/min to at or about 2 mg/min, such as at or about 0.1.0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0 mg/min, or a range defined by any of the foregoing.

In some embodiments, the continuous administration of the psychedelic is at a dose of at or about 0.01 to at or about 0.2 mg/kg, at a rate of at or about 0.1 mL/kg/h to at or about 5 mL/kg/h. In some embodiments, the continuous administration of the psychedelic is at 10 mg/kg at the rate of 5 mL/kg/h.

In some aspects, the maintenance dose is administered over a period of at or about 10 minutes to at or about 120 minutes, such as at or about 10, 20, 30, 40, 45, 50, 60, 90 or 120 minutes, or a range defined by any of the foregoing. In some embodiments, the maintenance dose is administered over a period of at or about 30, 60, 90 or 120 minutes, or a range defined by any of the foregoing. In some embodiments, the maintenance dose is administered over a period of up to at or about 60 minutes. In some embodiments, the maintenance dose is administered over a period of up to at or about 120 minutes. In some aspects, the maintenance dose is administered over at or about 40 minutes. In some aspects, the maintenance dose is administered over at or about 45 minutes. In some aspects, the maintenance dose is administered over at or about 50 minutes. In some aspects, the maintenance dose is administered over at or about 55 minutes.

In some embodiments, both the loading dose and the maintenance dose are administered by intravenous infusion. In some aspects, the loading dose, for example when administered by an intravenous infusion, has a higher infusion rate than the maintenance dose. In some aspects, the loading dose is administered by intravenous infusion at a rate of at or about 0.5 mL/min to at or about 2.0 mL/min, such as at or about 1.0 mL/min; and the maintenance dose is administered by intravenous infusion at a rate of at or about 0.1 mL/min to at or about 1.0 mL/min, such as at or about 0.5 mL/min.

In some aspects, the loading dose is administered as an initial bolus injection, and the maintenance dose is administered by continuous administration, such as by intravenous infusion. In some aspects, the loading dose of a psychedelic by intravenous administration of an initial bolus in an amount between at or about 1 mg and at or about 5 mg, such as at or about 1 mg, and the maintenance dose is administered by intravenous infusion at a rate of at or about 0.02 mg/min to at or about 1 mg/min.

In some embodiments, the provided methods and uses involve the administration of a loading dose of a psychedelic by intravenous administration of an initial bolus in an amount between at or about 1 mg and at or about 5 mg. In some aspects, the loading dose is an amount of a psychedelic sufficient to induce a dissociative state in the subject. In some aspects, the loading dose is at or about 1 mg. In some embodiments, a maintenance dose of the psychedelic is administered afterwards, for example, intravenous infusion at a rate of at or about 0.02 mg/min to at or about 1 mg/min to maintain the mean plasma concentration during a therapeutic window. In some aspects, the maintenance dose is administered at a rate of at or about 0.5 mg/min.

In some embodiments, the methods and uses involve administering to a subject having a psychological disorder a loading dose of a psychedelic (e.g., psilocybin, psilocin, a co-crystal, a co-former, or a salt thereof, or a combination thereof) by intravenous administration of an initial bolus in an amount between at or about 1 mg and at or about 5 mg; and thereafter continuously administering a maintenance dose of the psychedelic by intravenous infusion at a rate of at or about 0.02 mg/min to at or about 1 mg/min to maintain the mean plasma concentration during a therapeutic window. In some aspects, the maintenance dose is administered at a rate of at or about 0.5 mg/min.

In some embodiments, the provided methods and uses involve the administration of a loading dose of a psychedelic by intravenous infusion at a rate of at or about 0.5 mL/min to at or about 2.0 mL/min over a period of at or about 5 minutes to at or about 20 minutes. In some aspects, the loading dose is an amount of a psychedelic sufficient to induce a dissociative state in the subject. In some aspects, the loading dose is administered by intravenous infusion at a rate of at or about 1.0 mL/min over a period of at or about 10 minutes. In some embodiments, a maintenance dose of the psychedelic is administered afterwards, for example, by intravenous infusion 0.1 mL/min to at or about 1.0 mL/min over a period of at or about 30 minutes to at or about 120 minutes, such as at or about 30, 40, 45, 50, 60, 70, 80, 90, 100, 110, or 120 minutes, or a range defined by any of the foregoing. In some aspects, the maintenance dose is administered at a rate of at or about 0.5 mL/min for at or about 60 minutes.

In some aspects, the provided methods and uses involve administering to a subject having a psychological disorder a loading dose of a psychedelic (e.g., psilocybin, psilocin, a co-crystal, a co-former, or a salt thereof, or a combination thereof) by intravenous infusion at a rate of at or about 0.5 mL/min to at or about 2.0 mL/min over a period of at or about 5 minutes to at or about 20 minutes; and thereafter continuously administering a maintenance dose of the psychedelic by intravenous infusion at a rate of at or about 0.1 mL/min to at or about 2.0 mL/min over a period of at or about 30 minutes to at or about 120 minutes, to maintain the mean plasma concentration during a therapeutic window. In some aspects, the maintenance dose is administered at a rate of at or about 0.5 mL/min for at or about 60 minutes.

In some aspects, the therapeutic window is between at or about 30 minutes to at or about 120 minutes. In some aspects, the therapeutic window is at or about 60 minutes. In some aspects, the therapeutic window is at or about 120 minutes.

In some aspects, the total amount of psychedelic that is administered to the subject is up to between at or about 1.0 mg to at or about 20 mg per subject, such as at or about 1.0 mg, 1.5 mg. 2.5 mg, 5 mg, 7.5 mg, 10 mg, 15 mg or 20 mg per subject, or a range defined by any of the foregoing. In some embodiments, the total amount of psychedelic that is administered to the subject is up to at or about 2.5 mg. In some embodiments, the total amount of psychedelic that is administered to the subject is up to at or about 5 mg. In some embodiments, the total amount of psychedelic that is administered to the subject is up to at or about 10 mg.

In some embodiments, the psychedelic is psilocin, and the total amount of psilocin that is administered to the subject is up to between at or about 1 mg to at or about 10 mg, such as at or about 2.5 mg, and the administration of psilocin is carried out over a period of between at or about 30 minutes to 120 minutes, such as at or about 60 minutes.

In some embodiments, the psychedelic is psilocybin, and the total amount of psilocybin that is administered to the subject is up to between at or about 1 mg to at or about 10 mg, such as at or about 5 mg, and the administration of psilocybin is carried out over a period of between at or about 30 minutes to 120 minutes, such as at or about 60 minutes.

terminating administration of the psychedelic to terminate the dissociative state. In some aspects, the terminating administration involves discontinuing the intravenous administration, such as discontinuing an intravenous infusion. In some aspects, the termination of dissociative state occurs within at or about 30, 60, 90, 120, or 180 minutes after the termination of administration of the psychedelic, or a range defined by any of the foregoing. In some aspects, the termination of dissociative state occurs within at or about 60 minutes after the termination of administration of the psychedelic. In some aspects, the termination of the dissociative state can be measured or observed using one or more non-invasive measurements of brain activity, such as any described herein, e.g., EEG, and/or pharmacokinetic parameters, such as plasma concentration, Cₘₐₓ or AUC.

In some embodiments, one or more non-invasive measurements of brain activity, such as any described herein, e.g., EEG, and/or pharmacokinetic parameters, such as plasma concentration, Cₘₐₓ or AUC, are indicative of or associated with the entry into and/or exit from the dissociative state. In some aspects, one or more non-invasive measurements of brain activity, such as EEG, and/or pharmacokinetic parameters can be used to monitor the entry and exit from the dissociative state, and also to tailor, customize, change or adjust the dosage, rate or timing of the administration based on the measurements.

Accordingly, intravenous administration of the psychedelic results in a rapid and controllable entry into the dissociative state, in as little as 10 minutes after administration of an initial dose. This is in contrast to the administration of oral psilocybin, which once administered orally gives no control over the plasma levels of psilocybin or psilocin. This contributes to significant inter-subject variation. Accordingly, the methods provided herein, particularly those that involve inducing a dissociative state then maintaining a predetermined plasma concentration of the psychedelic allow minimizing or avoiding adverse events, control the plasma level of the psychedelic and thereby increase the safety and reproducibility of the methods.

Conventional treatments using psychedelics involve a lag time of up to 3 hours prior to the subject entering the dissociative state, which can last for up to an additional 6 hours, which is well beyond a useful therapeutic window. Accordingly, the ability to initiate the dissociative state more rapidly and withdrawing the subject from a dissociative state has the advantage that the treatment window can be controlled by the treating physician thereby minimising the total treatment time.

In some aspects, by closely controlling the level of psychedelic administered to the subject, the methods can avoid some of the adverse effects of psychedelic use associated with high plasma concentrations of psychedelics such as psychosis, negative emotional experiences (a ‘bad trip’) or tachycardia, increases in blood pressure, vasoconstriction, vasospasm, transient, delayed headache, rhabdomyolysis, transient renal failure or insufficiency, transient loss or reduction of visual acuity, ‘flashbacks’, diaphoresis, combativeness, and hallucinogen persisting perception disorder (HPPD).

In some embodiments, the methods described herein reduce the incidence of adverse effects by up to 100% compared to conventional treatment with oral psychedelics. For example, the methods can reduce the incidence of adverse effects by at or about 5%, 10%, 15%, 20%, 15%, 30%, 35%, 40%, 45%, 50%, 55%, 60%, 65%, 70%, 75%, 80%, 85%, 90%, 95% or 100% compared to conventional treatment. Adverse effects include psychotic, increased blood pressure, tachycardia, nausea, vomiting, ataxia, muscle weakness, increased anxiety, panic, paranoia, terror or the subject reporting a ‘bad trip’.

The dissociative state can be assessed subjectively, for example by the therapist providing psychological support. In some embodiments, entry into the dissociative state and/or monitoring of the duration of the dissociative state uses a technique for non-invasive measurement of brain activity such as electroencephalography (EEG) functional magnetic resonance imaging (fMRI), near-infrared spectroscopy (NIRS), magnetoencephalography (MEG), optoencephalography (OEG). In some aspects, any of the non-invasive measurement of brain activity described herein can be used to assess and monitor the entry to, status and/or exit from the dissociative state. Preferably, EEG is used.

Psychological support is provided to the subject after induction of the dissociative state. In the context of the methods described herein, psychological support is any form of support that is aimed at helping the subject to enhance their mental health and their cognitive, emotional and behavioural well-being. Typically, psychological support is provided by a therapist, psychiatrist, or other suitably qualified healthcare professional and preferably by a therapist who has been specifically trained on the administration of psychedelics.

In some embodiments, once the psychological support has been provided, the therapeutic window can be closed by withdrawing the subject from the dissociative state. This can be achieved in a number of ways such as by ceasing to administer the psychedelic or by ceasing to administer the psychedelic together with administration of a 5-HT_{2A}R antagonist. Suitable 5-HT_{2A}R antagonists include ketanserin, ritanserin, setoperone, olanzapine, mirtazapine, and mianserin. Compounds that have combined dopamine and 5-HT_{2A}R antagonist activity, such as risperidone, may also be used.

There is also provided a method of determining the rate and amount of a psychedelic to be administered to a subject to customize dosing regimens for an individual subject.

This method comprises administering a therapeutically effective dose of a psychedelic (such as psilocybin or psilocin) that is sufficient to induce a dissociative state in the subject, preferably within at least 10 minutes of the administration. An EEG is then obtained from the subject to confirm that the subject has entered a dissociative state. In some embodiments EEG monitoring may begin before or at around the same time as the psychedelic is administered.

The EEG monitoring may thereafter be continuous or intermittent to allow monitoring of the subject while further doses of the psychedelic are administered to maintain the mean plasma concentration of the psychedelic at a predetermined value to maintain the dissociative state during a therapeutic window.

### B. Psychological Support

In some embodiments, the provided methods and uses involve providing psychological support, e.g., psychotherapy, in addition to or in conjunction with administration of the psychedelic, such as psilocin or psilocybin. In some aspects, the methods or uses involve administration of psychedelic and psychological support or mental health support, e.g., psychotherapy, to a subject, such as a human subject. In some aspects, the methods or uses involve administration of psychedelic and psychotherapy to an individual in need thereof. In some aspects, the methods or uses involve administration of psychedelic and talk therapy to an individual in need thereof. In some embodiments, psychological support or mental health support occurs in-person and/or remotely, such as by phone or video conference. In some embodiments, psychotherapy occurs in-person and/or remotely, such as by phone or video conference. In some embodiments, talk therapy occurs in-person and/or remotely, such as by phone or video conference.

In some embodiments, psychological support comprises psychotherapy and/or talk therapy. In some embodiments, psychological support is therapist-led or led by a person or persons other than the individual. In some embodiments, psychological support is self-administered. In some embodiments, psychological support is therapist-led and self-administered. In some embodiments, psychological support precedes administration of the psychedelic, such as during a preparation session. In some embodiments, psychological support precedes administration of the psychedelic. In some embodiments, psychological support accompanies administration of the psychedelic. In some embodiments, psychological support follows or occurs after administration of the psychedelic, such as during an integration session. In some embodiments, psychological support follows after administration of the psychedelic.

In some embodiments, the methods or uses involve psychological support conducted in single-individual sessions, wherein one individual meets with one or more therapist. In some embodiments, the methods or uses involve psychological support conducted in single-individual sessions, wherein one individual meets with two or more therapists. In some embodiments, the methods or uses involve psychological support conducted in group sessions, wherein more than one individual meets with one or more therapists. In some embodiments, one or more of the individual's family members or friends may be present at the one or more pre-administration psychological support session. In some embodiments, the one or more psychological support sessions is conducted in person or remotely, such as by phone or video conference.

Due to the psychoactive nature of the psychedelic, the safety of subjects in clinical studies can be enhanced by testing the psychedelic within a “set and setting” protocol (Lyons and Carhart-Harris, J Psychopharmacol 2018; 32(7):811-19). In some aspects, “set” relates to one or more of the emotional, cognitive, behavioral state, mindset, and expectations of subjects prior to, such as immediately prior, to the psychedelic administration. In some aspects, “setting” relates to the physical environment in which administration of the psychedelic occurs. By addressing the set and setting of the experience, the risk of an individual reporting a distressing event or injuring themselves can be reduced. In some embodiments, this approach comprises three components: 1) psychological support prior to the psychedelic administration (e.g., preparation), 2) administration, and 3) post-administration psychological support to integrate the classic hallucinogen experience (e.g., integration).

In some embodiments, prior to the administration, subjects undergo pre-exposure preparation sessions comprising rapport building with the therapists who would be present during the drug administration session. In some embodiments, prior to the first dose, subjects undergo pre-exposure preparation sessions comprising identifying personal themes and struggles that might be especially likely to impact the session experience. In some embodiments, the drug administration session itself is conducted by one therapist. In some embodiments, the drug administration session itself is conducted by two therapists, such as a male and a female therapist, who are present throughout the session. In some embodiments, sessions are typically conducted in a room designed to be quiet, comfortable, and aesthetically pleasing. In some embodiments, subjects are encouraged to wear eyeshades and listen to a program of music through headphones during the drug administration session to aid them in focusing their attention inward.

In some embodiments, the methods or uses involve supervising an individual until an observer judges that the effects of the psychedelic have completely subsided. In some embodiments, the methods or uses involve discharge criteria. In some embodiments, one criterion must be met for an individual to be discharged. In some embodiments, more than one criterion must be met for an individual to be discharged. In some embodiments, all criteria must be met for an individual to be discharged. In some embodiments, discharge criteria comprise one or more of whether a responsible friend or family member is available to accompany the individual home, whether the individual's blood pressure and heart rate have returned to pre-drug levels, whether the individual is deemed by an observer to be free of any acute drug effects, whether the individual believes they have returned to their psychological baseline, whether the observer(s) judge that it is safe to discharge the individual, and whether the individual expresses a readiness to go home.

### II. Monitoring of Dissociative State

In some aspect, provided herein are methods of inducing a dissociative state and/or treatment of a psychological disorder in a subject by administration of a psychedelic, for example by intravenous administration, that also involves monitoring of the dissociative state, such as the entry into or exit from the dissociative state, using one or more non-invasive measurements of brain activity. In some aspects, one or more non-invasive measurements of brain activity is indicative of or associated with the entry into and/or exit from the dissociative state. In some aspects, one or more non-invasive measurements of brain activity can be used to monitor the entry and exit from the dissociative state, and also to tailor, customize, change or adjust the dosage, rate or timing of the administration based on the measurements. In some aspects, the methods also relate to a method of treating a psychological disorder, a disease having a significant psychological component and/or diseases or indications that relate to pain or sensation of pain.

In some aspects, provided are methods of inducing a dissociative state that involves administering to a subject an amount of a psychedelic (e.g., psilocin or psilocybin) sufficient to induce a dissociative state in the subject; and obtaining a non-invasive measurement of brain activity from the subject to determine when the subject enters the dissociative state. In some aspects, provided are methods of inducing a dissociative state that involves administering to a subject an amount of a psychedelic (e.g., psilocin or psilocybin) sufficient to induce a dissociative state in the subject; and obtaining a measurement of electroencephalography (EEG) from the subject to determine when the subject enters the dissociative state.

In some aspects, also provided are methods of determining a therapeutically effective dose of a psychedelic (e.g., psilocin or psilocybin) that involves administering to a subject an amount of a psychedelic sufficient to induce a dissociative state in the subject; obtaining a non-invasive measurement of brain activity (e.g., EEG) from the subject to determine if the subject enters the dissociative state; and determining the amount of the psychedelic as a therapeutically effective dose if the subject enters the dissociative state. In some aspects, the therapeutically effective dose is determined based on the non-invasive measurement of brain activity (e.g., EEG).

In some aspects, also provided are a method of maintaining a dissociative state in a subject that involves administering to the subject an amount of a psychedelic (e.g., psilocin or psilocybin) sufficient to induce a dissociative state in the subject less than 30 minutes after administration; and obtaining a measurement of electroencephalography (EEG) from the subject to determine when the subject enters the dissociative state; and thereafter maintaining the mean plasma concentration of the psychedelic at a predetermined value to maintain the dissociative state during a therapeutic window.

In some embodiments, the non-invasive measurement of brain activity is obtained prior to, during and/or after administration. In some aspects, the provided embodiments also involve obtaining a further non-invasive measurement of brain activity from the subject to monitor the dissociative state during the therapeutic window. In some aspects, a further measurement of EEG is obtained from the subject to monitor the dissociative state during the therapeutic window. In some aspects, the non-invasive measurement of brain activity after administration indicates the termination of dissociative state.

### A. Non-Invasive Measurement of Brain Activity

Provided herein are methods that involve a non-invasive measurement of brain activity to monitor the dissociative state of a patient having been administered a psychedelic (e.g., psilocin or psilocybin). In some embodiments, the non-invasive measure of brain activity includes one or more of an electroencephalography (EEG), a functional magnetic resonance imaging (fMRI), a near-infrared spectroscopy (NIRS), a magnetoencephalography (MEG), and an optoencephalography (OEG), quantitative sensory testing (QST), proton magnetic resonance spectroscopy (¹H-MRS), diffusion tensor imaging (DTI), questionnaire evaluation of psychedelic experience, and combinations thereof. In some embodiments, the non-invasive measure of brain activity includes one or more of an electroencephalography (EEG), a functional magnetic resonance imaging (fMRI), a near-infrared spectroscopy (NIRS), a magnetoencephalography (MEG), and an optoencephalography (OEG). In some embodiments, the non-invasive measure of brain activity includes an electroencephalography (EEG). In some embodiments, the non-invasive measure of brain activity includes questionnaire evaluation of psychedelic experience.

In some aspects, exemplary non-invasive methods for measuring brain activity, such as electroencephalography (EEG), functional magnetic resonance imaging (fMRI), near-infrared spectroscopy (NIRS), magnetoencephalography (MEG), or optoencephalography (OEG), can be used to monitor and assess the subject and the entry and/or the exit from the dissociative state or the psychedelic state, in accordance with the provided methods and uses. In some aspects, other exemplary non-invasive methods for measuring brain activity includes quantitative sensory testing (QST), proton magnetic resonance spectroscopy (¹H-MRS), diffusion tensor imaging (DTI) and combinations thereof.

In some aspects, non-invasive methods have been used to measure brain-specific changes during development, aging, learning, disease, and in response to acute stimuli, including small-molecule compounds and/or biomolecules (Borsook et al., Nat Rev Drug Discov 2006; 5(5):411-24; Matthews and Hampshire, Neuron 2016; 91(3):511-28; Carmichael et al., Drug Discov Today 2018; 23(2):333-48). The non-invasive measurements of brain activity can be used to more closely assess, monitor and control the dissociative state and the psychedelic experience and psychedelic-based interventions, and can be used to measure changes and/or improvements during psychedelic intervention (e.g., administration of psilocin or psilocybin), for example, in accordance with the provided embodiments. In some aspects, the non-invasive measurement of brain activity is observation of behavior.

In some embodiments, exemplary parameters measured by non-invasive measurements of brain activity include an explosive synchronization assessment, a power spectrum analysis, a default mode network analysis, a functional connectivity and activation analysis, a glutamine plus glutamate measurement, a GABA measurement, and a grey matter volume assessment.

### 1. Electroencephalography (EEG)

In some of any of the provided embodiments, brain activity is measured to monitor the entry into and/or exit from a dissociative state, using non-invasive methods such as electroencephalography (EEG). EEG is a tool for non-invasive studies on neurological disorders (Neto and Rosa, Neurosci Biobehav Rev 2019; 105:83-93). EEG is both convenient and effective in neurological studies. EEG analysis records high-resolution temporal brain activity (Michel and Brunet, Front Neurol 2019; 10:325), measuring direct electrical signals via the scalp and providing millisecond timescale information, compared seconds and minutes needed for other methodologies. EEG data can be collected from multiple different states (e.g., during rest, during tonic cuff pressure, during visual stimulus) to capture objective response signals and changes in brain activity. EEG is limited to measuring the uppermost layer of the brain, but when paired with other brain activity measurements (e.g., an fMRI), EEG provides key data for generating a comprehensive view and quantitative assessment of brain activity (Abreu et al., Front Hum Neurosci 2018; 12:29). EEG data is often used in the diagnosis of neurological disorders such as seizures, epilepsy, head injuries, brain tumors, and sleep problems.

In some aspects, EEG data will depict measured electrical activity at the surface of the brain, which is recorded as waveforms of different frequency (hertz, Hz), amplitude (microvoltage, μV), and shape. The physical location of the electrode used for the recording is an important factor for EEG data interpretation. The most common EEG waveforms frequencies are delta, theta, alpha, beta, or gamma, measured in cycles per second or hertz (Hz). Although many features of an EEG are rhythmic, other features are transient, including spikes, sharp waves, vertex waves, and sleep spindles. The presence and absence of different wave frequencies in various parts of the brain, as well as the rhythmic nature of these waves, can be used to diagnose neurological disease and monitor ongoing treatment (e.g., administration of a psychedelic, such as psilocin or psilocybin) in psychopharmacology.

In some embodiments, EEG is utilized to evaluate the activity of a psychedelic (e.g., psilocin or psilocybin) in inducing a dissociative state or a psychedelic state, and/or in the treatment of a psychological disorder, for example, in accordance with the provided embodiments. In some aspects, exemplary indicators measured with EEG include explosive synchronization of network activity at rest and Power spectrum analysis (frequency bands).

In some aspects, analysis methods and tools for the analysis, quantitation and interpretation of EEG data, can be used to determine when the subject enters the dissociative state. Exemplary tools and methods include those described in, e.g., Burns et al., Version 1. F1000Res. 2015; 4: 137. Analysis of EEG data can be complicated due to the variation in stimuli (e.g., therapeutic intervention) and patient-specific differences (e.g., epilepsy, depression, Alzheimer's disease), but accounting for measures of complexity (i.e., different patterns in the EEG signal) during data interpretation has been found to be beneficial. Exemplary measures of complexity that can be used include Lempel-Ziv complexity (Lempel and Ziv, IEEE Trans Inform Theory 1976; 22(1): 75-81), fractal dimension estimation (FD), permutation entropy (PE), Wiener entropy (WE), and spectral structure variability (SSV) (see, e.g., Burns et al., Version 1. F1000Res. 2015; 4: 137). The Lempel-Ziv complexity (LZC) is an algorithm-based measure and a surrogate for neural signal diversity.

In some aspects, LZC is the number of identified different sub-strings encountered as the binary sequence (e.g., pre-processed EEG signal) is iteratively viewed as a stream. The LZC computes the number of different patterns present in a sequence of symbols. When a large number of different patterns are present, the sequence is considered complex and may be difficult to compress. LZC is obtained by dividing the number of different patterns by the maximum complexity of a sequence of length N (Hornero et al., Philos Trans A Math Phys Eng Sci 2009; 367(1887):317-36). To determine LZC, the time series is first reduced to a symbol list. The EEG signals are converted into binary sequences s=s(1), s(2) . . . s(N), where s(i)=0 if x(i)<Td and s(i)=1 otherwise, for example as described in Hornero et al., Philos Trans A Math Phys Eng Sci 2009; 367(1887):317-36. The threshold Td is chosen as the median of x, since the latter is robust to outliers. In some aspects, the measurement of EEG is analyzed as expressed by Lempel-Ziv complexity (LZC).

2. Functional Magnetic Resonance Imaging (fMRI)

In some aspects, the non-invasive measure of brain activity includes a functional magnetic resonance imaging (fMRI). In some aspects, an fMRI detects changes in blood flow within the brain, wherein these changes correspond to regional and site-specific changes in activity. In certain aspects, fMRI can be used to assess the entry into and/or exit from the dissociative state. In some aspects, functional connectivity MRI (fMRI) focuses on the default mode network (DMN) and salience network (SLN). The DMN is a constellation of brain regions engaged in self-referential cognition, which are “deactivated” during externally focused tasks (Buckner and Vincent, Neuroimage 2007; 37(4): 1091-99; Fox and Raichle Nat Rev Neurosci 2007; 8(9):700-11). In some aspects, subjects with more centralized pain display increased connectivity between the DMN and the insula (an SLN region) which is diminished with successful treatment (Napadow et al., Arthritis Rheum 2012; 64(7):2398-2403). Whole-brain BOLD functional images are acquired while subjects rest comfortably in the scanner with eyes closed for about 8 minutes at the beginning of the scan session to obtain baseline measures of functional brain connectivity. Immediately following, subjects undergo an fMRI scan with sensory stimuli (e.g., pain) to evaluate functional brain connectivity response to the stimuli.

### 3. Proton Magnetic Resonance Spectroscopy (¹H-MRS)

1H-MRS is a non-invasive neuroimaging technique that enables the detection, identification, and quantification of metabolites and biochemical compounds in the brain tissue (Castillo et al., AJNR Am J Neuroradiol 1996; 17(1): 1-15; Sidek et al., Eur Radiol 2016; 26(12):4404-12). In some cases, with respect to indications related to pain, using proton magnetic resonance spectroscopy (1H-MRS), reports indicate that changes in levels of glutamine plus glutamate (Glx) and gamma aminobutyric acid (GABA) within specific regions of the brain (e.g., the posterior and anterior insula) are strongly correlated with improvements in pain symptoms, wherein reductions in clinical pain is associated with reduced Glx levels and elevated GABA levels (Mawla et al., Arthritis Rheumatol 2021; 73(7): 1318-28; Harris et al., Arthritis Rheum 2009; 60(10):3146-52; Harris et al., Arthritis Rheum 2008; 58(3):903-7). 1H-MRS provides metrics that can be used in longitudinal studies, wherein high-resolution anatomical scans isolate identical brain structures within individuals over time thus minimizing error that otherwise would occur because of slight differences in voxel location from one evaluation to the next. 1H-MRS is directed to the right posterior insula cortex both prior to and after stimulus. Individuals receiving treatment are at rest during the 1H-MRS session, wherein one or more ¹H-MRS assessment is integrated into the fMRI neuroimaging workflow.

### 4. Diffusion Tensor Imaging (DTI)

Diffusion tensor imagining (DTI) is a method for anatomical mapping and characterization of microstructures in the brain (Alexander et al., Neurotherapeutics 2007; 4(3):316-29), making it useful for bridging the gap between EEG data and fMRI data, collectively enhancing the value of the neurological data set. DTI leverages the fMRI platform and used for characterizing small changes in brain structure during neuropathology and treatment (Alexander et al., Neurotherapeutics 2007; 4(3):316-29; Soares et al., Fron Neurosci 2013; 7:31). DTI data is collected during the fMRI neuroimaging procedures and can aid with spatial mapping of EEG data. Unique structural brain networks are generated for each individual receiving treatment to better inform prior and subsequent EEG analyses. In some embodiments, the DTI assessment is utilized to supplement or enhance other neuroimaging results for evaluating the effect of psychedelic in entry into and exit from the dissociative state.

### 5. Quantitative Sensory Testing (QST)

Quantitative sensory testing (QST), a psychophysical testing approach that includes the determination of thresholds or stimulus response curves for sensory processing under normal and pathological conditions (e.g., psychological disorders such as pain related diseases), wherein the stimulus is quantified and used to measure perception (Yarnitsky and Granot, Handb Clin Neurol 2006; 81:397-409). QST is a semi-subjective (e.g., assessing subjective responses) technique, but responses are derived using known stimulus inputs, providing an objective framework for assessing the individual (e.g., before, during, or after therapeutic intervention, such as administration of the psychedelic). Examples of stimulus modalities include electrical, thermal, immersion thermal, mechanical, chemical, and light touch, wherein the pain measurements might include pain threshold/tolerance, temporal summation, conditioned pain modulation, pain rating, pain area mapping, cerebral responses (e.g., EEG, fMRI, PET), or muscle reflexes (Uddin and MacDermid, Pain Med 2016; 17(9): 1694-703). All QST procedures have been evaluated for safety and are well tolerated by subjects with pain (Petersen et al., Pain 2015; 156(1):55-61; Goodin et al., J Pain 2009; 10(2):180-90; Weissman-Fogel et al., J Pain 2009; 10(6):628-36; Price et al., Pain 2002; 99(1-2):49-59).

### 6. Questionnaire-Based Evaluation of the Psychedelic Experience

In some aspects, the entry and exit from the dissociative state, the psychedelic experience and the associated clinical impacts are evaluated using questionnaires, before, during and/or after the administration of the psychedelic in accordance with the provided embodiments. In some aspects, to evaluate the psychedelic experience and the associated clinical impacts for an individual receiving treatment in accordance with the provided embodiments, the individual is subject to a one or more questionnaire evaluation to assess the psychedelic experience and clinical activity in response to treatment (Davis et al., J Psychopharmacol 2021; 35(4):437-46). In some embodiments, the exemplary questionnaire evaluation to assess the psychedelic experience and clinical activity in response to treatment includes one or more of: the mystical experience questionnaire (MEQ30) evaluation, the challenging experience questionnaire (CEQ) evaluation, the psychological insight questionnaire (PIQ) evaluation, a qualitative written assessment, and/or the monitor rating scale (MRS) questionnaire. In some embodiments, the questionnaire-based evaluation of psychedelic experience is utilized to monitor the dissociative state, and the psychedelic experience of an individual receiving the psychedelic. In some embodiments, the questionnaire-based evaluation of the psychedelic experience is selected from among one or more of: the mystical experience questionnaire (MEQ30) evaluation, the challenging experience questionnaire (CEQ) evaluation, the psychological insight questionnaire (PIQ) evaluation, a qualitative written assessment, and/or the monitor rating scale (MRS) questionnaire.

a. Mystical Experience Questionnaire (MEQ30)

The mystical experience questionnaire (MEQ or MEQ30; derived and abbreviated from the MEQ43) is an evaluation completed by the patient, comprising four factors: mystical (e.g., internal unity, external unity, noetic quality, and sacredness scales), positive mood, transcendence of time and space, and ineffability (Barrett et al., J Psychopharmacol 2015; 29(11): 1182-90). The MEQ30 predicts persisting therapeutic benefits (e.g., change in attitudes, behavior, and well-being) attributed to psychedelic experiences, serving as a key measure of an individual's mystical experience. There are 30 items included in the MEQ30 evaluation, distributed across the four factors, and each items is rated on a 0-5 point scale, wherein 0 is “none; not at all,” 1 is “so slight cannot decide,” 2 is “slight,” 3 is “moderate,” 4 is “strong (equivalent in degree to any previous strong experience or expectation of this description),” and 5 is “extreme (more than ever before in my life and stronger than 4).” Across these 30 items, the four factors, or sub-categories, comprise different items, wherein mystical consists of items 4-6, 9 14-16, 18, 20, 21, 23-26, and 28; positive mood consists of items 2, 8, 12, 17, 27, and 30; space/time consists of items 1, 7, 11, 13, 19, and 22; and ineffability consists of items 3, 10, and 29. Scores for each item within a sub-category, or across the full MEQ30 set, are summed together and converted to a percentage by dividing by the corresponding point total. A “complete experience” is defined by a MEQ30 score equal to or exceeding 60% (of the total) across all four sub-categories, wherein the complete experience correlates with more significant and/or extended therapeutic benefits.

b. Challenging Experiences Questionnaire (CEQ)

Due to the powerful experience induced by classical hallucinogens (e.g., psilocybin), a patient receiving treatment may endure one or more acute adverse psychological reactions (i.e., a bad trip, or a challenging experience). In some aspects, evidence support that symptoms including affective (panic, depressed mood), cognitive (confusion, feelings of losing sanity), and somatic (nausea, heart palpitation) are possible (Barrett et al., J Psychopharmacol 2016; 30(12): 1279-95). Understanding the range and severity of symptoms is useful in tailoring patient care and anticipating patient needs during future treatment sessions. The challenging experience questionnaire (CEQ) addresses seven prominent factors (grief, fear, death, insanity, isolation, physical distress, and paranoia) and provides a phenomenological profile of challenging aspects that a patient has or may experience during treatment (Barrett et al., J Psychopharmacol 2016; 30(12): 1279-95). There are 26 items included in the CEQ evaluation, and each response carries a numerical value, ranging from 0-5, wherein 0 is ‘none; not at all’ and 5 is ‘extreme (more than ever before in my life).’ Across these 26 items, the seven factors, or sub-categories, comprise different items, wherein grief consists of items 2, 6, 9, 11, 23, and 25; fear consists of items 4, 7, 14, 21, and 26; death consists of items 16 and 20; insanity consists of items 8, 13, and 19; isolation consists of items 1, 10, and 24; physical distress consists of items 3, 5, 15, 17, and 18; and paranoia consists of items 12 and 22. Responses are summed within each sub-category and converted to a percentage of the total possible points. Further, responses for all item are summed and converted to a percentage of the total possible points.

c. Psychological Insight Questionnaire (PIQ)

In the context of psychedelic treatment, existing measures of insight fail to capture the acute insight associated a psychedelic-induced experience. To address this need, the psychological insight questionnaire (PIQ) was created, a new tool for assessing the intensity of a broad range of acute insights (Davis et al., J Psychopharm 2021; 35(4):437-46). The PIQ is a 23-item evaluation comprising two subscales, wherein one assesses insights into avoidance and maladaptive patterns (AMP), while the other assesses goals and adaptive patterns (GAP) (Davis et al., J Psychopharm 2021; 35(4):437-46). Items 1, 3, 5, 7, 9, 10, 12, 14, 15, 17-19, 21, and 23 relate to the AMP subscale, while items 2, 4, 6, 8, 11, 13, 16, 20, and 20 relate to the GAP subscale. Individuals respond to each item using a 6-point scale from 0 to 5, wherein 0 is “no, not at all.” 3 is “moderately.” and 5 is “extremely (more than ever before in my life).” Responses are summed and averaged, for each subscale and in total, with a higher score for each corresponding to higher levels of acute insight experience. PIQ, AMP, and GAP scores correlate well with increases in psychological flexibility, well-being, and long-term positive outcomes.

d. Monitor Rating Scale (MRS)

The monitor rating scale (MRS) questionnaire is completed by a clinician and involves rating and scoring dimensions of subject's behavior and/or mood during the treatment session (Griffiths et al., Psychopharmacology Berl 2006; 187(3):268-83). There are 20 items (or dimensions) included in the MRS evaluation, and some dimension are assigned a numerical value, ranging from 0-4, wherein 0 is ‘none; no effect’ and 4 is ‘peak effect; happening often.’ For other dimensions, scoring is assigned as the total duration (in minutes, across a defined window of time) observed for a patient behavior (e.g., talking with the clinician or total speech).

e. Qualitative (Written) Assessment

A subjective narrative description of the psychedelic experience can provide insight into unique aspects, needs and concerns for specific individuals during and/or after treatment completion. Narrative descriptions are written after each treatment dose session (e.g., first or second dose). After the final dosing session, individuals participate in therapy integration sessions (e.g., one hour each week), wherein discussion of the one or more narrative description helps the individual to gain insight and understanding from their experience. The narrative description is also used to investigate common themes associated with this therapy. In some cases, individuals are asked open-ended questions during the 3 month follow-up to gauge persistent changes in behavior, thought patterns, and emotions in response to receiving treatment.

### 7. Behavioral Responses

In some aspects, the non-invasive measure of brain activity includes observing behavioral aspects of the subject. Exemplary behaviors that can be observed include behaviors similar to a head-twitching response (HTR), for example, as observed in an animal such as in rodents. Exemplary HTR observation include those described in, for example, Zhuk et al., Toxins 2015, 7, 1018-1029; Halberstadt et al., J Psychopharmacol. 2011 25(11): 1548-1561.

### III. Psychedelic Compounds and Compositions

In some aspects, the provided embodiments involve the administration of a psychedelic. In some aspects, the psychedelic is psilocybin, psilocin, a co-crystal, a co-former, or a salt thereof, or a combination thereof. In some aspects, the psychedelic is psilocin, a co-crystal, co-former, or salt thereof. In some aspects, the psychedelic is psilocin. In some aspects, the psychedelic is psilocybin, a co-crystal, co-former, or salt thereof. In some aspects, the psychedelic is psilocybin.

As used herein, the terms ‘psychedelic compound’, ‘psychedelic drug’, ‘psychedelic substance’ and ‘psychedelic’ are used interchangeably to refer to an agonist of the serotonin 2A receptor (5-HT_{2A}R). There is a strong positive correlation between a psychedelic's affinity for the 5-HT_{2A}R and its psychedelic potency.

Psychedelics suitable for use in the methods described herein include baeocystin, norbaeocystin, N,N-dimethyltryptamine, 5-hydroxytryptamine (serotonin), 5-hydroxytryptophan, psilocybin, psilocin, their precursors and immediate derivatives. Synthetic or natural prodrugs, congeners and analogs of psilocybin, psilocin, baeocystin and norbaeocystin may also be used.

Congeners are chemical substances related to each other by origin, structure, or function. Analogs (or analogues or structural analogs) are compounds having a structure similar to another but differing from it in respect of a certain substituent in which one or more atoms or functional groups, which are replaced with other atoms or groups or substituents.

Psilocybin, psilocin and analogs that may similarly be useful include those where the hydroxyl group is modified, or the methyl groups of the terminal amine nitrogen have been modified. Example hydroxyl group substituents include alkyl and aryl ethers and esters, for example methoxy and ethoxy ethers and acetyl esters, halogens including fluoro-, chloro- and bromo-substituents, and thio groups such as methylthio or benzothio. Example nitrogen group substituents include one or both methyl groups substituted with ethyl, propyl, isopropyl, butyl, isobutyl, secbutyl, tertbutyl, amyl or allyl groups and the N-trimethyl analogs. The corresponding phosphate esters, namely psilocybin analogs, may also prove useful, as may analogs where one or more hydrogen atom is replaced by fluorine, chlorine, or bromine.

In some embodiments, the salts of the compounds are used, for example hydrochloride, fumarate, maleate, picrate, oxalate, tartrate, and sulfate salts, which are typically more stable. The salts can be zwitterionic forms, particularly of the phosphate esters and quaternary ammonium compounds.

Examples of suitable analogs include: 4-acetoxy-N,N-dimethyltryptamine (4-AcO-DMT or O-acetyl psilocin) the acetylated form of the psilocin (4-OH-DMT) a compound is a potential prodrug of psilocin (as are other 4-alkyl-esters), more stable than psilocin, and has a longer shelf life; 4-acetoxy-N-methyl-N-ethyltryptamine (4-AcO-MET), a psilocin analog substituted at R4 of its indole heterocycle with an acetoxy (AcO or CH₃COO—) functional group which also contains a methyl group and an ethyl chain bound to the terminal amine nitrogen of its tryptamine backbone. 4-AcO-MET is an acetate ester analog of 4-OH-MET and a N-substituted ethyl homolog of 4-AcO-DMT; 4-acetoxy-N,N-diethyltryptamine (4-AcO-DET); 4-acetoxy-N-methyl-N-propyltryptamine (4-AcO-MPT); 4-acetoxy-N-methyl-N-isopropyltryptamine (4-AcO-MIPT); 4-acetoxy-N,N-dipropyltryptamine (4-AcO-DPT) and 4-acetoxy-N,N-diisopropyltryptamine (4-AcO-DI PT); 4-hydroxy-N-methyl-N-ethyltryptamine (4-OH-MET, metocin, or methylcybin), a 4-hydroxy N-substituted structural analog of psilocin and the with a methyl and an ethyl group bound to the terminal amine nitrogen of the tryptamine structure; 4-hydroxy-N-methyl-N-propyltryptamine (4-OH-MPT); 4-hydroxy-N-methyl-N-isopropyltryptamine (4-OH-MI PT); 4-hydroxy-N,N-diethyltryptamine (4-OH-DET); 4-hydroxy-N,N-dipropyltryptamine (4-OH-DPT); 4-hydroxy-N,N-diisopropyltryptamine (4-OH-DI PT); and 4-hydroxy-N,N-diallyltryptamine (4-OH-DALT); analogs where the 4-OH group has been removed, such as N,N-dimethyltryptamine (DMT), N-methyl-N-ethyltryptamine (MET), N-methyl-N-propyltryptamine (MPT), N,N-diethyltryptamine (DET), N,N-dipropyltryptamine (DPT), N,N-isopropyltryptamine (DIPT), N-methyl-N-isopropyltryptamine (MIPT), α-methyltryptamine (AMT), N-ethyl-N-isopropyltryptamine (EIPT), N-methyl-N-butyl-tryptamine (MBT) or analogs substituted at other positions such as N,N-dimethyl-5-hydroxytryptamine (5-OH-DMT or bufotenine), 5-methoxy-α-methyltryptamine (5-MeO-aMT), 5-methoxy-N,N-dimethyltryptamine (5-MeO-DMT), 5-methoxy-N,N-diethyltryptamine (5-MeO-DET), 5-methoxy-N,N-dipropyltryptamine (5-MeO-DPT), 5-methoxy-N,N-diisopropyltryptamine (5-MeO-DIPT), 5-methoxy-N-ethyl-N-isopropyltryptamine (5-MeO-EI PT), 2,α-dimethyltryptamine (2,α-DMT), α,N-dimethyltryptamine (α,N-DMT), α-ethyltryptamine (α-ET), 2-methyl-N,N-dimethyltryptamine (2-Me-DMT), 2-methyl-N,N-diethyltryptamine (2-Me-DET), 1-methylpsilocin and ibogaine (a complex tryptamine).

In some embodiments, equimolar amounts of a psilocin or psilocybin analog may be used in place of psilocybin and/or psilocin, or amounts producing equivalent functional effects may be used.

In one embodiment, the psychedelic is psilocybin. Psilocybin is a naturally occurring psychedelic compound produced by more than 200 species of mushrooms, collectively known as psilocybin mushrooms. Once administered to a subject, psilocybin is metabolized by endogenous alkaline phosphatase to psilocin, which then acts on serotonin receptors in the brain. In a preferred embodiment, the psychedelic is psilocin.

Psilocybin is also known as [3-(2-dimethylaminoethyl)-1H-indol-4-yl] dihydrogen phosphate, and given the CAS number 520-52-5. Psilocin (also known as 4-hydroxy-N,N-dimethyltryptamine (4-HO-DMT), psilocine, psilocyn, or psilotsin) and given the CAS number 520-53-6.

Co-crystals are multicomponent systems in which two components, an active pharmaceutical ingredient and a conformer, are present in stoichiometric ratio and bonded together in the crystal lattice by hydrogen bonding, π-π interactions, and van der Waals forces. In some embodiments the conformer and the active can be bonded with a non-covalent bond such as a halogen bond.

Examples of suitable coformers include, acetamide, benzamide, (+/−)-limonene, 1-(phenylazo)-2-naphthylamine, 1,2,6-hexanetriol, 1,2-dimyristoyl-sn-glycero-3-(phospho-s-(1-glycerol)), 1,2-dimyristoyl-sn-glycero-3-phosphocholine, 1,2-diolcoyl-sn-glycero-3-phosphocholine, 1,2-dipalmitoyl-sn-glycero-3-(phospho-rac-(1-glycerol)), 1,2-distearoyl-sn-glycero-3-(phospho-rac-(1-glycerol)), 1,2-distearoyl-sn-glycero-3-phosphocholine, 1,5-naphthalene-disulfonic acid, 1-hydroxy-2-naphthoic acid, 1-o-tolylbiguanide, 2-ethyl-1,6-hexanediol, 4-aminobenzoic acid, 4-aminopyridine, 4-aminosalicylic acid, 4-chlorobenzene-sulfonic acid, 4-ethoxyphenyl urea, 7-oxo-dhea, acacia, acacia mucilage, acacia syrup, acesulfame, acesulfame potassium, acetohydroxamic acid, acetone sodium bisulfite, acetylated lanolin alcohols, acetylated monoglycerides, acetylcysteine, acetyltributyl citrate, acrylates copolymer, acrylic acid-isooctyl acrylate copolymer, adenine, adipic acid, alanine, albumin aggregated, albumin colloidal, albumin human, albumins, alginic acid, alkyl ammonium sulfonic acid betaine, alkyl aryl sodium sulfonate, allantoin, allopurincol, allyl alpha-ionone, alpha-terpineol, alpha-tocopherol, alpha-tocopherol acetate, aminobenzoate sodium, amyl acetate, anethole, anhydrous citric acid, anhydrous dextrose, anhydrous lactose, anhydrous tribasic sodium phosphate, anhydrous trisodium citrate, arginine, arlacel, asafetida, ascorbic acid, ascorbyl palmitate, asparagine, aspartame, aspartic acid, bacteriostatic sodium chloride injection, barium sulfate, benzalkonium chloride, benzenesulfonic acid, benzethonium chloride, benzododecinium bromide, benzoic acid, benzyl acetate, benzyl alcohol, benzyl benzoate, benzyl chloride, beta-carotene, betanaphthol, betose, bibapcitide, bismuth subcarbonate, bismuth subgallate, boric acid, brocrinat, butyl stearate, butylated hydroxyanisole, butylated hydroxytoluene, butylparaben, butyric acid, C-11-1-aminocyclohexanecarboxylic acid, C12-15 alkyl lactate, caffeine, calcobutrol, caldiamide sodium, caloxetate trisodium, calteridol calcium, camphoric acid, capric acid, captan, captisol, carboxypolymethylene, carmine, carnauba wax, carnauba yellow wax, carrageenan, carrageenan calcium, carrageenan salt, carrageenan sodium, ceresin, ceteareth-12, ceteareth-15, ceteareth-30, cetearyl alcohol/ceteareth-20, cetearyl ethylhexanoate, ceteth-10, ceteth-2, ceteth-20, ceteth-23, cetostearyl alcohol, cetrimonium chloride, cetyl alcohol, cetyl esters wax, cetyl palmitate, cetylpyridinium chloride, chlorocresol, chloroxylenol, cholesterol, chrysin, cinnamaldehyde, cinnamic acid, citrate, citric acid, citric acid monohydrate, clemizole, cocamide ether sulfate, cocamine oxide, coco betaine, coco diethanolamide, coco monoethanolamide, coco-caprylate, coco-glycerides, creatine, creatinine, cresol, cupric sulfate, cyclamic acid, cyclomethicone, cyclomethicone 5, cysteine, dalfampridine, decyl methyl sulfoxide, dehydroacetic acid, denatonium benzoate, deoxycholic acid, dextran, dextran 40, dextrates, dextrin, dextrose, dextrose monohydrate, diacetylated monoglycerides, diatrizoic acid, dibasic anhydrous sodium phosphate, dibasic sodium phosphate, dibasic sodium phosphate dihydrate, dibasic sodium phosphate dodecahydrate, dibasic sodium phosphate heptahydrate, dibutyl phthalate, dibutyl sebacate, diethyl phthalate, diethyl pyrocarbonate, diethyl sebacate, diethylaminoethyl stearamide phosphate, diethylene glycol monoethyl ether, diethylene glycol monomethyl ether, diethylhexyl phthalate, diisopropyl adipate, diisopropyl dilinoleate, diisopropylbenzothiazyl-2-sulfonamide, dimethicone medical fluid 360, dimethyl isosorbide, dimethyl phthalate, dimethyl sulfoxide, dimethyldioctadecylammonium bentonite, dimethylglycine, dimethylsiloxane/methylvinylsiloxane copolymer, dinoseb-ammonium, dipropylene glycol, disodium cocoamphodiacetate, disodium hydrogen citrate, disodium laureth sulfosuccinate, disodium lauryl sulfosuccinate, disodium oleamido monoethanolamine sulfosuccinate, disodium sulfosalicylate, disofenin, dl-a350 lactic acid, dl-acetyltryptophan, dl-alpha-tocopherol, dl-alpha-tocopherol acetate, dl-dipalmitoylphosphatidylglycerol, dl-distearoylphosphatidylcholine, dl-glutamic acid, dl-tartaric acid, d-mannose, dmdm hydantoin, docosanol, docusate sodium, d-ribose, edetate calcium disodium, edetate disodium, edetate sodium, edetic acid, egg phosphatidyl glycerol, egg phospholipids, entsufon, entsufon sodium, epilactose, epitetracycline hydrochloride, erythorbic acid, erythritol, ethanolamine hydrochloride, ethyl maltol, ethyl oleate, ethyl vanillate, ethyl vanillin, ethylenediamine dihydrochloride, ethylhexyl hydroxystearate, ethylparaben, eucalyptol, eugenol, exametazime, fatty acid esters, fatty acid glycerides, fatty acid pentaerythriol ester, fatty acids, fatty alcohol citrate, fatty alcohols, ferric chloride, ferric oxide, ferrosoferric oxide, ferrous fumarate, ferrous oxide, fluorescein, fructose, fumaric acid, fumaryl diketopiperazine, gadolinium oxide, galactaric acid, galactose, gamma cyclodextrin, genistein, gentisic acid, gentisic acid ethanolamide, gentisic acid ethanolamine, gluceptate sodium, gluconic acid, gluconolactone, glucosamine, glucose, glucuronic acid, glutamic acid, glutamic acid hydrochloride, glutamine, glutaric acid, glutathione, glyceryl caprylate, glyceryl dibehenate, glyceryl distearate, glyceryl isostearate, glyceryl laurate, glyceryl monostearate, glyceryl oleate, glyceryl palmitate, glyceryl palmitostearate, glyceryl ricinoleate, glyceryl stearate, glyceryl stearate-laureth-23, glyceryl stearate/peg stearate, glyceryl stearate/peg-100 stearate, glyceryl stearate/peg-40 stearate, glyceryl stearate-stearamidoethyl diethylamine, glyceryl trioleate, glycine, glycine hydrochloride, glycol distearate, glycol stearate, glycolic acid, glycyrrhizin, guanidine hydrochloride, hexylresorcinol, hippuric acid, histidine, hyaluronate sodium, hydrocortisone, hydroquinone, hydrous-citric acid, hydroxyethylpiperazine ethane sulfonic acid, hydroxyoctacosanyl hydroxystearate, hydroxyprogesterone caproate, hydroxypropyl beta-cyclodextrin, hystrene, illicium anisatum, imidazole, imidurea, indigotindisulfonate sodium, iodoxamic acid, iofetamine hydrochloride, ipriflavone, isoleucine, isopropyl isostearate, isopropyl myristate, isopropyl myristate-myristyl alcohol, isopropyl palmitate, isopropyl stearate, isostearic acid, isostearyl alcohol, lactate, lactitol monohydrate, lactobionic acid, lactose, landalgine, lanolin, lauralkonium chloride, lauramine oxide, laureth sulfate, lauric acid, lauric diethanolamide, lauric myristic diethanolamide, lauroyl sarcosine, lauryl lactate, lauryl sulfate, lecithin, leucine, levomenthol, levulinic acid, lidofenin, I-sodium lactate, lysine, maleic acid, malic acid, malonic acid, maltitol, maltodextrin, maltol, maltose anhydrous, mandelic acid, mannitol, maprofix, mebrofenin, medium-chain triglycerides, medronate disodium, medronic acid, menthol, metacresol, methionine, methyl salicylate, methyl stearate, methylchloroisothiazol inone, methylisothiazoli none, methylparaben, methylparaben sodium, miripirium chloride, mono and diglyceride, monobasic sodium phosphate, monobasic sodium phosphate anhydrous, monobasic sodium phosphate dihydrate, monobasic sodium phosphate monohydrate, monoglyceride citrate, monoglycerides, monosodium citrate, monosodium glutamate, monostearyl citrate, monothioglycerol, myristic acid, myristyl alcohol, myristyl lactate, niacinamide, nicotinamide, nicotinic acid, N-methyl glucamine, octanoic acid, oleth-20, oleyl alcohol, oleyl oleate, orotic acid, oxalic acid, oxidronate disodium, oxyquinoline, palmitamine oxide, palmitic acid, pamoic acid, pentadecalactone, pentaerythritol cocoate, pentasodium pentetate, pentetate calcium trisodium, pentetic acid, phenol, phenonip, phenoxyethanol, phenylalanine, phenylethyl alcohol, phospholipid, piperazine, piperazine hexahydrate, procaine, product wat, proline, propenyl guaethol, propyl gallate, propylene carbonate, propylene glycol, propylene glycol-lecithin, propylene glycol alginate, propylene glycol diacetate, propylene glycol dicaprylate, propylene glycol monolaurate, propylene glycol monopalmitostearate, propylene glycol palmitostearate, propylene glycol ricinoleate, propylene glycol/diazolidinyl urea/methylparaben/propylparben, propylparaben, propylparaben sodium, p-toluenesulfonic acid, pyridoxamine, pyridoxine (4-pyridoxic acid), quercetin, resveratrol, riboflavin, saccharin, saccharin calcium, saccharin sodium, saccharin sodium anhydrous, salicylic acid, saturated fatty acid esters, sebacic acid, serine, sodium 1,2-ethanedisulfonate, sodium 2-naphthalenesulfonate, sodium acetate, sodium acetate anhydrous, sodium alginate, sodium alkyl sulfate, sodium aluminum silicate, sodium ascorbate, sodium benzoate, sodium bicarbonate, sodium bisulfate, sodium bisulfate acetone, sodium bisulfite, sodium bitartrate, sodium borate, sodium borate decahydrate, sodium carbonate, sodium carbonate decahydrate, sodium carbonate monohydrate, sodium carboxymethyl beta-glucan (ds 065-085), sodium caseinate, sodium cellulose, sodium cetostearyl sulfate, sodium chlorate, sodium chloride, sodium chloride injection, sodium cholesteryl sulfate, sodium citrate, sodium citrate hydrous, sodium cocoyl sarcosinate, sodium cyclamate, sodium desoxycholate, sodium dithionite, sodium dodecylbenzenesulfonate, sodium ethylparaben, sodium formaldehyde sulfoxylate, sodium gluconate, sodium hydroxide, sodium hypochlorite, sodium iodide, sodium lactate, sodium laureth-2 sulfate, sodium laureth-3 sulfate, sodium laureth-5 sulfate, sodium lauroyl sarcosinate, sodium lauryl sulfate, sodium lauryl sulfoacetate, sodium metabisulfite, sodium nitrate, sodium oleate, sodium phosphate, sodium phosphate dihydrate, sodium phosphite, sodium polyacrylate, sodium polyacrylate (2500000 MW), sodium polymetaphosphate, sodium propionate, sodium pyrophosphate, sodium pyrrolidone carboxylate, sodium starch glycolate, sodium starch glycolate type a corn, sodium starch glycolate type a potato, type B potato sodium starch glycolate, sodium stearate, sodium stearyl fumarate, sodium succinate hexahydrate, sodium sulfate, sodium sulfate anhydrous, sodium sulfate decahydrate, sodium sulfite, sodium sulfosuccinated undecyclenic monoalkylolamide, sodium tartrate, sodium thioglycolate, sodium thiomalate, sodium thiosulfate, sodium thiosulfate anhydrous, sodium trimetaphosphate, sodium tripolyphosphate, sodium xylenesulfonate, sorbic acid, sorbitan, sorbitan isostearate, sorbitan monolaurate, sorbitan monooleate, sorbitan monopalmitate, sorbitan monostearate, sorbitan sesquioleate, sorbitan trioleate, sorbitan tristearate, sorbitol, squalane, stannous 2-ethylhexanoate, stearalkonium chloride, stearalkonium hectorite/propylene carbonate, stearamidoethyl diethylamine, stearates, stearic acid, stearic diethanolamide, stearoxytrimethylsilane, stearyl alcohol, succinic acid, sucralose, sucrose, sucrose distearate, sucrose laurate, sucrose palmitate, sucrose polyesters, sucrose stearate, sucrose syrup, sulfacetamide sodium, sulfobutylether beta-cyclodextrin, tagatose, tartaric acid, tegacid, tert-butylhydroquinone, tetrofosmin, theophylline, thimerosal, threonine, thymol, tocopherol, tocophersolan, tragacanth, triacetin, tribasic sodium phosphate, tribasic sodium phosphate monohydrate, tribehenin, tricaprylin, triceteareth-4 phosphate, triethanolamine lauryl sulfate, triethyl citrate, trihydroxystearin, trilaneth-4 phosphate, trilaureth-4 phosphate, trimyristin, tris, trisodium citrate dihydrate, trisodium hedta, tristearin, trolamine, tromantadine, tromethamine, tryptophan, tyloxapol, tyrosine, undecylenic acid, urea, urethane, ursodiol, valine, vanillin, versetamide, viscarin, vitamin E, vitamin E acetate, vitamin K5, xylitol, and zinc sulfate.

Since the formation of co-crystals can control solubility, water adsorption, dissolution rate, and other physicochemical properties of drugs, many studies on co-crystals of drugs have been conducted to improve the effects of drugs. Co-crystals of drugs are can be manufactured and can improve drug properties such as solubility, stability and processability of poorly soluble drugs.

Psilocybin and psilocin are used in a powder form characterized by poor stability of the active and storage of psilocybin formulations is problematic. Accordingly, there is provided a psilocybin or psilocin cocrystal with improved stability and solubility. There is also provided a method for producing the psilocybin or psilocin cocrystal.

Psilocybin and psilocin cocrystals as described herein comprise at least one conformer, which can form supramolecular synthons with the psilocybin molecule. The formation of these supramolecular synthons allows cocrystals to form.

In one embodiment, the coformer has a carboxylate moiety, which can form an acid amide heterosynthon with the nitrogen atoms in the psilocybin or psilocin molecule. In another embodiment, the coformer molecule forms heterosynthons with the phosphate moiety of psilocybin.

Also provided is a method for preparing psilocybin or psilocin cocrystal for the methods comprises providing psilocybin or psilocin molecule with at least one coformer molecule. The psilocybin or psilocin and at least one conformer is mixed to prepare a mixed solution. Co-crystals can be formed from the mixed solution by evaporative crystallization, cooling crystallization, reaction crystallization, and isothermal crystallization.

Evaporative co-crystallization involves the nucleation and growth of a cocrystal from the mixed solution with supersaturation provided by removal of the solvent from the solution via evaporation. Individual cocrystals, or the bulk crystal sample, should be harvested before the solution evaporates to dryness to ensure recovery of a clean crystals.

Cocrystals may also be formed by solid-state grinding which is a method were solid materials that will result in a cocrystal are admixed in stoichiometric amounts, are pressed and crushed together with mortar and pestle, ball mix or other grinding type millery.

Liquid assisted grinding is a modification of solid-state grinding by the addition of a small amount of solvent during the grinding process, and is used to enhance supramolecular selectivity, both polymorphic and stoichiometric in the crystalline system. This method enhances the co-crystallization rate, as some cocrystals showed poor performance in cocrystal formation under solid-state grinding conditions.

Cooling crystallization can be used to prepare co-crystals with supersaturation provided by a temperature drop.

In another embodiment, the co-crystals may be formed by isothermal slurry conversion. This involves the suspension of the psilocybin or psilocin and the conformer in a solvent with the solid fraction always remaining in excess. Typically this involves adding the psilocybin or psilocin to a solution or suspension of coformer in solvent. While this is a solution based method, it does not require that the mixed solution contain fully dissolved psilocybin or psilocin and conformer.

Alternatively, co-crystals can be obtained by a supercritical fluid method, for example using supercritical carbon dioxide (CO₂). In these methods, the supercritical CO₂ is used to suspend the psilocybin or psilocin and the coformer as a slurry in liquid or supercritical CO₂. By controlling the thermodynamic conditions of CO₂ (e.g., temperature, pressure), it is possible to fine-tune its density and solvent power, which provides control over the co-crystallization between cocrystal components.

In one embodiment, co-crystals can be obtained by using a non-solvent different from the solvent used in preparing the mixed solution.

In some embodiments, the psilocybin or psilocin co-crystal has improved solubility stability or both compared to psilocybin or psilocin alone.

The methods can utilize the psychedelic, a pharmaceutically acceptable salt thereof. The term “pharmaceutically acceptable salt” refers to those salts which, within the scope of sound medical judgement, are suitable for use in contact with the tissues of humans and animals without undue toxicity, irritation, allergic response and the like, and are commensurate with a reasonable benefit/risk ratio. Pharmaceutically acceptable salts are well known in the art. S. M. Berge et al. describe pharmaceutically acceptable salts in detail in J. Pharmaceutical Sciences, 1977, 66:1-19. For a review on suitable salts, see Handbook of Pharmaceutical Salts: Properties, Selection, and Use by Stahl and Wermuth (Wiley-VCH, 2002). Methods for making pharmaceutically acceptable salts of compounds of the invention are known to one of skill in the art. The salts can be prepared in situ during the final isolation and purification of the compounds of the invention, or separately by reacting the free base function with a suitable organic acid. Suitable pharmaceutically acceptable acid addition salts of the compounds of the present invention may be prepared from an inorganic acid or from an organic acid. Examples of such inorganic acids are hydrochloric, hydrobromic, hydroiodic, nitric, carbonic, sulfuric, and phosphoric acid. Appropriate organic acids may be selected from aliphatic, cycloaliphatic, aromatic, heterocyclic carboxylic and sulfonic classes of organic acids, examples of which are formic, acetic, propionic, succinic, glycolic, gluconic, lactic, malic, tartaric, citric, ascorbic, glucoronic, fumaric, maleic, pyruvic, alkyl sulfonic, arylsulfonic, aspartic, glutamic, benzoic, anthranilic, mesylic, methanesulfonic, salicylic, p-hydroxybenzoic, phenylacetic, mandelic, ambonic, pamoic, pantothenic, sulfanilic, cyclohexylaminosulfonic, stearic, algenic, β-hydroxybutyric, galactaric, and galacturonic acids. Suitable pharmaceutically acceptable base addition salts of the compounds of the present invention include metallic salts made from lithium, sodium, potassium, magnesium, calcium, aluminum and zinc, and organic salts made from organic bases such as choline, diethanolamine, morpholine. Alternatively, suitable pharmaceutically acceptable base addition salts of the compounds of the present invention include organic salts made from N,N′-dibenzylethylenediamine, chloroprocaine, choline, diethanolamine, ethylenediamine, meglumine (N-methylglucamine), procaine, ammonium salts, quaternary salts such as tetramethylammonium salt, amino acid addition salts such as salts with glycine and arginine. In the case of compounds that are solids, it will be understood by those skilled in the art that the compounds, agents and salts may exist in different crystalline or polymorphic forms, all of which are intended to be within the scope of the present invention.

The psychedelic compounds, salts thereof, or co-crystals described herein may be administered as a formulation or composition comprising a pharmaceutically effective amount of the compound, in association with one or more pharmaceutically acceptable excipients including carriers, vehicles and diluents, chemical stabilizers such as anti-oxidants. The term “excipient” herein means any substance, not itself a therapeutic agent, used as a diluent, adjuvant, or vehicle for delivery of a therapeutic agent to a subject or added to a pharmaceutical composition to improve its handling or storage properties or to permit or facilitate formation of a dosage form such as a solution or suspension suitable for parenteral administration such as by intravenous, intramuscular, intradermal, or subcutaneous, nasal or buccal application.

Suitable salts include any pharmaceutically acceptable salt that can be prepared from an inorganic acid or from an organic acid. Examples of such inorganic acids are hydrochloric, hydrobromic, hydroiodic, nitric, carbonic, sulfuric, boric, sulfamic, and phosphoric acid. Appropriate organic acids may be selected from aliphatic, cycloaliphatic, aromatic, heterocyclic carboxylic and sulfonic classes of organic acids, examples of which are formic, acetic, propionic, succinic, butyric, glycolic, gluconic, lactic, mucic, malic, tartaric, citric, ascorbic, glucoronic, fumaric, maleic, edetic, hydroxymaleic, pyruvic, alkyl sulfonic, arylsulfonic, aspartic, glutamic, benzoic, palmitic, oleic, lauric, valeric, benzenesulfonic, oxalic, anthranilic, mesylic, methanesulfonic, toluenesulfonic, salicylic, p-hydroxybenzoic, phenylacetic, mandelic, ambonic, pamoic, pantothenic, tannic, sulfanilic, cyclohexylaminosulfonic, stearic, algenic, β-hydroxybutyric, galactaric, and galacturonic acids. Suitable pharmaceutically acceptable base addition salts of the compounds of the present invention include metallic salts made from lithium, sodium, potassium, magnesium, calcium, aluminum and zinc, and organic salts made from organic bases such as choline, diethanolamine, morpholine. Alternatively, suitable pharmaceutically acceptable base addition salts of the compounds of the present invention include organic salts made from N,N′-dibenzylethylenediamine, chloroprocaine, choline, diethanolamine, ethylenediamine, meglumine (N-methylglucaminc), procaine, ammonium salts, alkylamonnium, quaternary salts such as tetramethylammonium salt, amino acid addition salts such as salts with glycine and arginine. Also, basic nitrogen-containing groups may be quaternized with such agents as lower alkyl halides, such as methyl, ethyl, propyl and butyl chlorides, bromides and iodides; dialkyl sulfates such as dimethyl and diethyl sulfate. Metal salts can be prepared by reaction of a psychedelic with a metal hydroxide. An acid salt can be prepared by reacting an appropriate acid with a psychedelic.

Excipients can include, by way of illustration and not limitation, diluents, wetting agents, polymers, lubricants, glidants, stabilizers, and substances added to improve appearance of the composition. Acceptable excipients include (but are not limited to) stearic acid, magnesium stearate, magnesium oxide, sodium and calcium salts of phosphoric and sulfuric acids, magnesium carbonate, talc, gelatin, acacia gum, sodium alginate, pectin, dextrin, mannitol, sorbitol, lactose, sucrose, starches, gelatin, cellulosic materials, such as cellulose esters of alkanoic acids and cellulose alkyl esters, low melting wax, cocoa butter or powder, polymers such as polyvinyl-pyrrolidone, polyvinyl alcohol, and polyethylene glycols, and other pharmaceutically acceptable materials. Examples of excipients and their use is described in Remington's Pharmaceutical Sciences, 20th Edition (Lippincott Williams & Wilkins, 2000). The choice of excipient will to a large extent depend on factors such as the particular mode of administration, the effect of the excipient on solubility and stability, and the nature of the dosage form.

In some embodiments, the compositions or formulations comprise an antioxidant such as pyruvate, ascorbate, or tartrate.

The compounds and pharmaceutical compositions of the invention may be formulated for injectable, nasal, parenteral, subcutaneous, intravenous, or intramuscular delivery. Non-limiting examples of particular formulation types include powders, granules, injectables, ampoules, vials, ready-to-use solutions, suspensions, or lyophilized materials.

For parenteral administration, including intravenous, intramuscular, or subcutaneous, administration, fluid unit dosage forms may be prepared by combining the psychedelic and a sterile vehicle, typically a sterile aqueous solution which is preferably isotonic with the blood of the subject. Depending on the vehicle and concentration used, the psychedelic may be either suspended or dissolved in the vehicle or other suitable solvent. In preparing solutions, the psychedelic may be dissolved in water for injection and filter sterilized before filling into a suitable vial or ampoule and scaling. Advantageously, agents such as a preservative and buffering agents can be dissolved in the vehicle. To enhance the stability, the composition may be frozen after filling into the vial and the water removed under vacuum. The dry lyophilized powder may then be sealed in the vial and an accompanying vial of water for injection or other suitable liquid may be supplied to reconstitute the liquid prior to use.

The total liquid volume before lyophilization can be less, equal to, or more than, the final reconstituted volume of the lyophilized formulation. The lyophilization process is well known to those of ordinary skill In the art, and typically Includes sublimation of water from a frozen formulation under controlled conditions. Lyophilized formulations typically can be stored at a wide range of temperatures. For example, lyophilized formulations may be stored below 25° C., for example, refrigerated at 2-8° C., or at room temperature (e.g., approximately 25° C.). Preferably, lyophilized formulations are stored below about 25° C., more preferably, at about 20° C.; below about 4° C.; or below about 0° C.

Lyophilized formulations are preferably reconstituted with a solution consisting primarily of water (e.g., USP WFI, or water for injection) or bacteriostatic water (e.g., USP WFI with 0.9% benzyl alcohol). Alternatively, solutions comprising buffers and/or excipients and/or one or more pharmaceutically acceptable carriers may be used. The liquid that is to undergo freeze-drying or lyophilization preferably comprises all components desired in a final reconstituted liquid formulation.

In some embodiments, there is provided a kit for use in the methods, the kit comprising the lyophilized formulation and optionally a solution for reconstitution of the lyophilized formulation and instructions for use.

The terms “combination therapy” or “adjunct therapy” in defining use of a psychedelic and one or more other pharmaceutical agents, are intended to embrace administration of each agent in a sequential manner in a regimen that will provide beneficial effects of the drug combination, and is intended as well to embrace co-administration of these agents in a substantially simultaneous manner, such as in a single formulation having a fixed ratio of these active agents, or in multiple, separate formulations of each agent.

In accordance with various embodiments of the present invention, one or more psychedelics may be formulated or administered in combination with one or more other therapeutic agents such as NSAIDs, or palmitoylethanolamide (PEA). Thus, in accordance with various embodiments of the present invention, one or more psychedelics may be included in combination treatment regimens with other known therapeutic agents or prophylactic agents.

Alternatively, the subject may be pre-treated with one or more other therapeutic agents such as NSAIDs, or palmitoylethanolamide (PEA). For example during the initial psychotherapy sessions which may begin start several weeks prior to administration of the psychedelic agent. In some embodiments treatment with one or more other therapeutic agents such as NSAIDs continues after the administration of the psychedelic.

Combination regimens may involve the active agents being administered together, sequentially, or spaced apart as appropriate in each case. Combinations of active agents including compounds of the invention may be synergistic.

The co-administration of compounds of the invention may be effected by the psychedelics being in the same unit dose as another active agent, or the compounds and one or more other active agent(s) may be present in individual and discrete unit doses administered at the same, or at a similar time, or at different times according to a dosing regimen or schedule. Sequential administration may be in any order as required, and may require an ongoing physiological effect of the first or initial compound to be current when the second or later compound is administered, especially where a cumulative or synergistic effect is desired.

For example, it is anticipated that when the methods described herein are used to treat fibromyalgia administration of an analgesic (such as an NSAID) to reduce pain as well as reducing potential CNS inflammation that may contribute to providing a positive environment that can be reinforced during the dissociative state. That is, in some embodiments there is a “pre-treatment period” to prepare the subject for administration of a psychedelic to enhance the impact of the psychedelic and to induce a more lasting effect which may otherwise be undermined by CNS inflammation.

In some embodiments there is a ‘pre-treatment period’ in which a therapeutic suitable for treating the that is used to prepare the brain for the psychedelic intervention enhancing the impact of the psychedelic to induce a more lasting effect which would not be undermined by CNS inflammation.

In some embodiments, the psychedelic (e.g. psilocybin or psilocin) can be administered with one or more of orlistat, lorcaserin, phenterminetopiramate, naltrexone-bupropion, liraglutide, phentermine, benzphetamine, diethylpropion, phendimetrazine, and SGLT2 inhibitors (glifozina). These combinations may be useful for treating eating disorders such as hypothalamic obesity, ROHHAD (rapid-onset obesity with hypothalamic dysregulation, hypoventilation, and autonomic dysregulation, binge eating, bulimia, anorexia, pica, rumination disorder, avoidant/restrictive food intake disorder (ARFID), purging disorder, night eating syndrome, and orthorexia.

In some embodiments, the psychedelic (e.g. psilocybin or psilocin) can be administered with one or more of pain relievers, antidepressants and antiseizure drugs for the treatment of fibromyalgia. In this context, suitable pain relievers such as acetaminophen, ibuprofen or naproxen sodium may be helpful. Gabapentin or pregabalin may also be used.

Suitable antidepressants include duloxetine and milnacipran (Savella) may help case the pain and fatigue associated with fibromyalgia. Suitable anti-seizure drugs include carbamazepina, clobazam, lamotrigine, levetiracetam, oxcarbazepine, phenytoin, sodium valproate, topiramate, lacosamide, and zonisamide.

In the context of fibromyalgia treatment, the psychedelic (e.g. psilocybin or psilocin) can alternatively or in addition, be co-administered with a muscle relaxant such as cyclobenzaprine, or co-administered with transcranial magnetic stimulation.

### IV. Psychological Disorders and Indications for Treatment

The methods and uses disclosed herein can be useful for the treatment or prevention of any psychological disorder, or disease having a significant psychological component. In some aspects, the psychological disorder or disease having a psychological component also includes diseases or indications that relate to pain or sensation of pain.

Examples of psychological disorders that may be treated or prevented by the methods disclosed herein include PTSD, alcohol and drug addiction, treatment resistant depression, anxiety, end of life anxiety (for example associated with a terminal disease such as cancer), eating disorders, fibromyalgia, neuropathic pain, phantom limb pain hypothalamic induced obesity (HO) and Prader-Willi syndrome (PWS), anorexia, bulimia, and binge-eating disorders. In some aspects, exemplary psychological disorders that can be treated in accordance with the provided embodiments include nociplastic pain.

In preferred embodiments, the methods described herein are used to treat hypothalamic induced obesity (HO), Prader-Willi syndrome (PWS), binge-eating or fibromyalgia.

Other examples of psychological disorders that may be treated or prevented by the methods disclosed herein include psychotic disorder, schizophrenia, schizophreniform disorder (acute schizophrenic episode); schizoaffective disorder; bipolar I disorder (mania, manic disorder, manic-depressive psychosis); bipolar II disorder; major depressive disorder with or without psychotic features (psychotic depression); delusional disorders (paranoia); shared psychotic disorder (shared paranoia disorder); brief psychotic disorder (other and unspecified reactive psychosis); psychotic disorder not otherwise specified (unspecified psychosis); paranoid personality disorder; schizoid personality disorder; schizotypal personality disorder, anxiety disorder, panic disorder, panic attacks, agoraphobia, and attention deficit syndrome.

In some aspects, the disorder or disease include pain related disorders, such as pain related disorders that are psychological disorder or disease having a psychological component. In some aspects, the pain related disorder includes pain related diseases that are associated with nociplastic pain or have nociplastic pain characteristics. In some aspects, the disease or disorder to be treated in accordance with the provided embodiments, e.g., the psychological disorder, includes nociplastic pain disorders.

In some aspects, nociplastic pain is not associated with signs of neuropathy but is characterized by hypersensitivity in apparently normal tissues. In some aspects, nociplastic pain results from altered nociception despite no clear evidence of actual or threatened tissue damage or evidence of a lesion or disease. In some aspects, nociplastic pain is chronic. Exemplary types of nociplastic pain include fibromyalgia, irritable bowel syndrome, phantom limb pain, complex regional pain syndrome, and non-specific chronic low back pain. In some aspects, nociplastic pain is mechanistically distinct from nociceptive pain, which is caused by ongoing inflammation and damage of tissues, and neuropathic pain, which is caused by nerve damage. The mechanisms that underlie nociplastic pain are not entirely understood, but could involve augmented central nervous system (CNS) pain and sensory processing and altered pain modulation. Exemplary symptoms observed in nociplastic pain include multifocal pain that is more widespread or intense, or both, than would be expected given the amount of identifiable tissue or nerve damage, as well as other CNS-derived symptoms, such as fatigue, sleep, memory, and mood problems. Nociplastic pain can occur in isolation, for example as in fibromyalgia or tension-type headache, or as part of a mixed-pain state in combination with ongoing nociceptive or neuropathic pain, as might occur in chronic low back pain (see, e.g., Fitzcharles et al. 2021 Lancet 397(10289):2098-2110).

In some embodiments, the psychological disorder or nociplastic pain disorder is fibromyalgia. In some embodiments, the psychological disorder or nociplastic pain disorder is phantom limb pain. In some embodiments, the psychological disorder or nociplastic pain disorder is complex regional pain syndrome.

In some aspects, an animal model of nociplastic pain can be used to assess the activity and effect of administration of psychedelics, for example, as described herein. In some examples, rats induced with pain by injection of formalin are used as an animal model of nociplastic pain. The formalin pain model is a translational animal model for chronic nociplastic pain (Vanini G., Sleep 2016; 39(1): 133-42), including fibromyalgia, phantom limb pain or complex regional pain syndrome. In the formalin pain model, in addition to experiencing extra pain in the formalin-injected paw, the animal experiences increased pain perception (i.e., hyperalgesia) in extremities not injected with formalin (e.g., the contra-lateral paw compared to the one injected with formalin). The pain perception was measured in the contra-lateral paw to assess the activity of psychedelics in nociplastic pain disorders.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### V. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Cₘₐₓ means the peak concentration that a drug achieves in the serum or plasma.

Throughout this specification, unless the context clearly requires otherwise, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Throughout this specification, the term ‘consisting of’ means consisting only of.

The term “consisting essentially of” means the inclusion of the stated element(s), integer(s) or step(s), but other element(s), integer(s) or step(s) that do not materially alter or contribute to the working of the invention may also be included.

Any discussion of documents, acts, materials, devices, articles or the like which has been included in the present specification is solely for the purpose of providing a context for the present technology. It is not to be taken as an admission that any or all of these matters form part of the prior art base or were common general knowledge in the field relevant to the present technology as it existed before the priority date of each claim of this specification.

Unless the context requires otherwise or specifically stated to the contrary, integers, steps, or elements of the technology recited herein as singular integers, steps or elements clearly encompass both singular and plural forms of the recited integers, steps or elements.

In the context of the present specification, the terms ‘a’ and ‘an’ are used to refer to one or more than one (i.e., at least one) of the grammatical object of the article. By way of example, reference to ‘an element’ means one element, or more than one element.

In the context of the present specification, the term ‘about’ means that reference to a figure or value is not to be taken as an absolute figure or value, but includes margins of variation above or below the figure or value in line with what a skilled person would understand according to the art, including within typical margins of error or instrument limitation. In other words, use of the term ‘about’ is understood to refer to a range or approximation that a person or skilled in the art would consider to be equivalent to a recited value in the context of achieving the same function or result.

The terms “treating”, “treatment” and “therapy” are used herein to refer to curative therapy, prophylactic therapy, palliative therapy and preventative therapy. Thus, in the context of the present disclosure the term “treating” encompasses curing, ameliorating, or tempering the severity of a psychological condition or one or more of its associated symptoms.

The terms “therapeutically effective amount” or “pharmacologically effective amount” or “effective amount” refer to an amount of an agent sufficient to produce a desired therapeutic or pharmacological effect in the subject being treated. The terms are synonymous and are intended to qualify the amount of each agent that will achieve the goal of improvement in disease severity and/or the frequency of incidence over treatment of each agent by itself while preferably avoiding or minimizing adverse side effects, including side effects typically associated with other therapies.

A “pharmaceutical carrier, diluent or excipient” includes, but is not limited to, any physiological buffered (i.e., about pH 7.0 to 7.4) medium comprising a suitable water soluble organic carrier, solvents, dispersion media, fillers, carriers, coatings, antibacterial and antifungal agents, isotonic and absorption delaying agents. Suitable water soluble organic carriers include, but are not limited to, saline, dextrose, corn oil, dimethylsulfoxide. Other additives include lactose, mannitol, corn starch, potato starch, binders such as microcrystalline cellulose, cellulose derivatives such as hydroxypropylmethylcellulose, acacia, gelatins, disintegrators such as sodium carboxymethylcellulose, and lubricants such as talc or magnesium stearate.

“Subject” includes any human.

In the context of this specification, the term “administering” and variations of that term including “administer” and “administration”, includes contacting, applying, delivering, or providing a compound or composition of the invention to a subject by any appropriate means.

Those skilled in the art will appreciate that the technology described herein is susceptible to variations and modifications other than those specifically described. It is to be understood that the technology includes all such variations and modifications. For the avoidance of doubt, the technology also includes all of the steps, features, and compounds referred to or indicated in this specification, individually or collectively, and any and all combinations of any two or more of said steps, features and compounds.

In order that the present technology may be more clearly understood, preferred embodiments will be described with reference to the following description.

### VI. EXEMPLARY EMBODIMENTS

Among the provided embodiments are:
1. A method of treating a psychological disorder in a subject, the method comprising
administering to a subject having a psychological disorder an amount of a psychedelic sufficient to induce a dissociative state in the subject less than 30 minutes after administration; and
thereafter maintaining the mean plasma concentration of the psychedelic at a predetermined value to maintain the dissociative state during a therapeutic window; wherein the psychedelic is psilocybin, psilocin, a co-crystal, a co-former, or a salt thereof, or a combination thereof.
2. The method of embodiment 1, wherein the method further comprises obtaining a non-invasive measurement of brain activity from the subject to determine when the subject enters the dissociative state.
3. A method of inducing a dissociative state, the method comprising:
administering to a subject having a psychological disorder, an amount of a psychedelic sufficient to induce the dissociative state in the subject; and
obtaining a non-invasive measurement of brain activity from the subject to determine when the subject enters the dissociative state; wherein the psychedelic is psilocybin, psilocin, a co-crystal, a co-former, or a salt thereof, or a combination thereof.
4. A method of inducing a dissociative state, the method comprising:
administering to a subject having a psychological disorder, an amount of a psychedelic sufficient to induce the dissociative state in the subject; and
obtaining a measurement of electroencephalography (EEG) from the subject to determine when the subject enters the dissociative state; wherein the psychedelic is psilocybin, psilocin, a co-crystal, a co-former, or a salt thereof, or a combination thereof.
5. A method of determining a therapeutically effective dose of a psychedelic, the method comprising:
administering to a subject having a psychological disorder, an amount of a psychedelic sufficient to induce a dissociative state in the subject;
obtaining a non-invasive measurement of brain activity from the subject to determine if the subject enters the dissociative state; and
determining the amount of the psychedelic as a therapeutically effective dose if the subject enters the dissociative state; wherein the psychedelic is psilocybin, psilocin, a co-crystal, a co-former, or a salt thereof, or a combination thereof.
6. A method of maintaining a dissociative state in a subject with a psychological disorder, the method comprising;
administering to the subject an amount of a psychedelic sufficient to induce the dissociative state in the subject less than 30 minutes after administration; and
obtaining a measurement of electroencephalography (EEG) from the subject to determine when the subject enters the dissociative state; and
thereafter maintaining a mean plasma concentration of the psychedelic at a predetermined value to maintain the dissociative state during a therapeutic window; wherein the psychedelic is psilocybin, psilocin, a co-crystal, a co-former, or a salt thereof, or a combination thereof.
7. A method of treating a psychological disorder, the method comprising:
performing the method of any one of embodiments 1-6; and
maintaining the mean plasma concentration of the psychedelic at a predetermined value to maintain the dissociative state during a therapeutic window; thereby treating the psychological disorder.
8. A method of treating phantom limb pain in a subject, the method comprising
administering to a subject having phantom limb pain an amount of a psychedelic sufficient to induce a dissociative state in the subject; wherein the psychedelic is psilocybin, psilocin, a co-crystal, a co-former, or a salt thereof, or a combination thereof.
9. The method of any one of embodiments 1-8, wherein the administration of the psychedelic is by intravenous administration.
10. The method of any one of embodiments 1-5 and 7-9, wherein the dissociative state is induced in the subject within less than at or about 15, 30, 60, 90 or 120 minutes after administration, or a range defined by any of the foregoing.
11. The method of any one of embodiments 1-5 and 7-10, wherein the dissociative state is induced in the subject within less than at or about 30 minutes after administration.
12. The method of any one of embodiments 3-5 and 8-11, wherein the method further comprises maintaining the mean plasma concentration of the psychedelic at a predetermined value to maintain the dissociative state during a therapeutic window.
13. The method of any one of embodiments 2, 3, 5, 7, and 9-12, wherein the non-invasive measurement of brain activity is selected from among electroencephalography (EEG), functional magnetic resonance imaging (fMRI), near-infrared spectroscopy (NIRS), magnetoencephalography (MEG), and optoencephalography (OEG).
14. The method of any one of embodiments 2, 3, 5, 7, and 9-13, wherein the non-invasive measurement of brain activity is EEG.
15. The method of any one of embodiments 2, 3, 5, 7, and 9-12, wherein the non-invasive measurement of brain activity is questionnaire-based evaluation of the experience.
16. The method of embodiment 15, wherein the questionnaire-based evaluation of the psychedelic experience is selected from among one or more of: the mystical experience questionnaire (MEQ30) evaluation, the challenging experience questionnaire (CEQ) evaluation, the psychological insight questionnaire (PIQ) evaluation, a qualitative written assessment, and/or the monitor rating scale (MRS) questionnaire.
17. The method of any one of embodiments 2, 3, 5, 7, and 9-16, wherein the method further comprises obtaining a further non-invasive measurement of brain activity from the subject to monitor the dissociative state during the therapeutic window.
18. The method of any one of embodiments 4, 6-12, and 14, wherein the method further comprises obtaining a further measurement of EEG from the subject to monitor the dissociative state during the therapeutic window.
19. The method of any one of embodiments 4, 6-12, 14, and 18, wherein the measurement of EEG is analyzed as expressed by Lempel-Ziv complexity (LZC).
20. The method of any one of embodiments 2, 3, 5, 7, and 9-17, wherein the non-invasive measurement of brain activity is obtained prior to, during and/or after administration.
21. The method of embodiment 20, wherein the non-invasive measurement of brain activity after administration indicates the termination of dissociative state.
22. The method of any one of embodiments 4, 6-12, 14, 18, and 19, wherein the measurement of EEG is obtained prior to, during and after administration.
23. The method of embodiment 22, wherein the measurement of EEG after administration indicates the termination of dissociative state.
24. The method of any one of embodiments 1-23, wherein the dissociative state is induced within at or about 1, 2, 3, 4, 5, 6, 7, 8, 9, 10, 11, 12, 13, 14, 15, 16, 17, 18, 19, or 20 minutes after the administration.
25. The method of any one of embodiments 1-24, wherein the dissociative state is induced within at or about 5 minutes after the administration.
26. The method of any one of embodiments 1, 6, 7, and 9-25, wherein the therapeutic window is at or about 0.5, 1, 2, 3, 4, 5, or 6 hours.
27. The method of any one of embodiments 1, 6, 7, and 9-26, wherein the therapeutic window is between at or about 0.5 hours and at or about 4 hours.
28. The method of any one of embodiments 1-27, further comprising terminating administration of the psychedelic to terminate the dissociative state.
29. The method of embodiment 28, wherein the termination of dissociative state occurs within at or about 30, 60, 90, 120, or 180 minutes after the termination of administration of the psychedelic, or a range defined by any of the foregoing.
30. The method of embodiment 28 or 29, wherein the termination of dissociative state occurs within at or about 60 minutes after the termination of administration of the psychedelic.
31. The method of any one of embodiments 1-30, wherein the psychedelic is administered to produce a predetermined Cₘₐₓ within at or about 30-120 minutes after the initiation of the administration.
32. The method of any one of embodiments 1-31, wherein the psychedelic is administered to produce a predetermined Cₘₐₓ within at or about 45-90 minutes after the initiation of the administration.
33. The method of any one of embodiments 1-30, wherein the psychedelic is administered to produce a predetermined Cₘₐₓ within at or about 1-10 minutes of the administration.
34. The method of any one of embodiments 30-33, wherein the Cₘₐₓ is at or about 1 μg/L-50 μg/L.
35. The method of any one of embodiments 30-34, wherein the Cₘₐₓ is at or about 10 μg/L-20 μg/L.
36. The method of any one of embodiments 30-35, wherein the Cₘₐₓ is at or about 10 μg/L-15 μg/L.
37. The method of any one of embodiments 1-36, wherein the dissociative state is induced by intravenous administration of a loading dose of the psychedelic.
38. The method of embodiment 37, wherein the loading dose comprises administration of an initial bolus of the psychedelic.
39. The method of embodiment 37 or 38, wherein the initial bolus dose is at or about 1 mg, 2 mg, 3 mg, 4 mg or 5 mg.
40. The method of any one of embodiments 37-39, wherein the loading dose of the psychedelic is administered over a period of at or about 1, 2, 3, 4, 5, 6, 7, 8, 9 or 10 minutes, or a range defined by any of the foregoing.
41. The method of any one of embodiments 37-40, wherein the loading dose of the psychedelic is administered over a period of at or about 3 minutes.
42. The method of any one of embodiments 38-41, wherein the initial bolus of psychedelic is at a dose of at or about 0.02 mg/kg to at or about 0.2 mg/kg.
43. The method of any one of embodiments 1-42, wherein the dissociative state is induced by intravenous administration of an initial bolus of the psychedelic at a dose of about 0.1 mg/kg to about 0.2 mg/kg.
44. The method of any one of embodiments 1, 6, 7, and 9-43, wherein the mean plasma concentration of the psychedelic is maintained at the predetermined value during the therapeutic window by administration of a maintenance dose of the psychedelic.
45. The method of embodiment 44, wherein the maintenance dose of the psychedelic is administered by continuous or intermittent administration of the psychedelic.
46. The method of embodiment 45, wherein the continuous or intermittent administration is via an intravenous route.
47. The method of any one of embodiments 44-46, wherein the maintenance dose is administered by an intravenous infusion.
48. The method of embodiment 45, wherein the maintenance dose of the psychedelic is administered by intermittent administration of the psychedelic and the intermittent administration is via a subcutaneous, oral, transdermal, intramuscular, intranasal, intranasal/pharanygeal, or buccal route.
49. The method of any one of embodiments 45-47, wherein the maintenance dose is administered by intravenous infusion of the psychedelic at a rate of at or about 0.2 mg/min to at or about 1 mg/min.
50. The method of any one of embodiments 45-47, wherein the continuous administration of the psychedelic is at a rate of at or about 0.1 mg/min to at or about 1 mg/min.
51. A method of treating a psychological disorder in a subject, the method comprising:
administering to a subject having a psychological disorder a loading dose of a psychedelic by intravenous administration of an initial bolus in an amount between at or about 1 mg and at or about 5 mg; and
thereafter continuously administering a maintenance dose of the psychedelic by intravenous infusion at a rate of at or about 0.02 mg/min to at or about 1 mg/min to maintain the mean plasma concentration during a therapeutic window; wherein the psychedelic is psilocybin, psilocin, a co-crystal, a co-former, or a salt thereof, or a combination thereof.
52. A method of treating a psychological disorder in a subject, the method comprising:
administering to a subject having a psychological disorder a loading dose of a psychedelic by intravenous infusion at a rate of at or about 0.5 mL/min to at or about 2.0 mL/min over a period of at or about 5 minutes to at or about 20 minutes; and
thereafter continuously administering a maintenance dose of the psychedelic by intravenous infusion at a rate of at or about 0.1 mL/min to at or about 2.0 mL/min over a period of at or about 30 minutes to at or about 120 minutes, to maintain the mean plasma concentration during a therapeutic window; wherein the psychedelic is psilocybin, psilocin, a co-crystal, a co-former, or a salt thereof, or a combination thereof.
53. The method of any one of embodiments 37-52, wherein the loading dose of the psychedelic is administered at a rate of at or about 0.1 mL/min to at or about 2.0 mL/min.
54. The method of any one of embodiments 37-53, wherein the loading dose of the psychedelic is administered at a rate of at or about 1.0 mL/min.
55. The method of any one of embodiments 44-54, wherein the maintenance dose is administered at a rate of 0.1 mL/min to at or about 1.0 mL/min.
56. The method of any one of embodiments 44-55, wherein the maintenance dose is administered at a rate of at or about 0.5 mL/min.
57. The method of any one of embodiments 44-56, wherein the maintenance dose is administered over a period of at or about 30, 60, 90 or 120 minutes, or a range defined by any of the foregoing.
58. The method of any one of embodiments 44-57, wherein the maintenance dose is administered over a period of up to at or about 60 minutes.
59. The method of any one of embodiments 44-58, wherein the maintenance dose is administered over a period of up to at or about 120 minutes.
60. The method of any one of embodiments 1, 6, 7, and 9-59, wherein the therapeutic window is between at or about 30 minutes to at or about 120 minutes.
61. The method of any one of embodiments 1, 6, 7, and 9-60, wherein the therapeutic window is at or about 60 minutes.
62. The method of any one of embodiments 1, 6, 7, and 9-60, wherein the therapeutic window is at or about 120 minutes.
63. The method of any one of embodiments 1-62, wherein the total amount of psychedelic that is administered to the subject is up to at or about 1.0 mg, 1.5 mg, 2.5 mg, 5 mg, 7.5 mg, 10 mg, 15 mg or 20 mg per subject, or a range defined by any of the foregoing.
64. The method of any one of embodiments 1-63, wherein the total amount of psychedelic that is administered to the subject is up to at or about 2.5 mg.
65. The method of any one of embodiments 1-63, wherein the total amount of psychedelic that is administered to the subject is up to at or about 5 mg.
66. The method of any one of embodiments 1-63, wherein the total amount of psychedelic that is administered to the subject is up to at or about 10 mg.
67. The method of any one of embodiments 1-66, wherein the psychedelic is psilocin, a co-crystal, a co-former, or a salt thereof.
68. The method of any one of embodiments 1-67, wherein the psychedelic is psilocin.
69. The method of any one of embodiments 1-66, wherein the psychedelic is psilocybin, a co-crystal, a co-former, or a salt thereof.
70. The method of any one of embodiments 1-66 and 69, wherein the psychedelic is psilocybin.
71. The method of any one of embodiments 1-68, wherein the psychedelic is psilocin, and the total amount of psilocin that is administered to the subject is up to at or about 2.5 mg, and the administration of psilocin is carried out over a period of at or about 60 minutes.
72. The method of any one of embodiments 1-66, 69, and 70, wherein the psychedelic is psilocybin, and the total amount of psilocybin that is administered to the subject is up to at or about 5 mg, and the administration of psilocybin is carried out over a period of at or about 60 minutes.
73. The method of any one of embodiments 1, 2, 6, 7, and 12-72, wherein the predetermined value of mean plasma concentration of the psychedelic is at or about 1-50 μg/L.
74. The method of any one of embodiments 1, 2, 6, 7, and 12-73, wherein the predetermined value of mean plasma concentration of the psychedelic is at or about 10-20 μg/L.
75. The method of any one of embodiments 1, 2, 6, 7, and 12-74, wherein the predetermined value of mean plasma concentration of the psychedelic is at or about 10-15 μg/L.
76. The method of any one of embodiments 1-75, wherein the method further comprises providing psychological support to the subject during the therapeutic window.
77. The method of any one of embodiments 1-76, wherein the method further comprises withdrawing the subject from the dissociative state at the end of the treatment window by administering a 5HT_{2A} receptor antagonist.
78. The method of any one of embodiments 1-7 and 9-77, wherein the psychological disorder is selected from the group consisting of PTSD, alcohol addition, drug addiction, treatment resistant depression, anxiety, end of life anxiety, an eating disorder, fibromyalgia, neuropathic pain, phantom limb pain, hypothalamic induced obesity, Prader-Willi syndrome, and binge-eating disorder.
79. The method of any one of embodiments 1-7 and 9-78, wherein the psychological disorder is selected from the group consisting of hypothalamic induced obesity, Prader-Willi syndrome, binge-eating disorder, and fibromyalgia.
80. The method of any one of embodiments 1-7 and 9-77, wherein the psychological disorder is a nociplastic pain disorder.
81. The method of any one of embodiments 1-7 and 9-80, wherein the psychological disorder is fibromyalgia.
82. The method of any one of embodiments 1-7 and 9-80, wherein the psychological disorder is phantom limb pain.
83. The method of any one of embodiments 1-7 and 9-80, wherein the psychological disorder is complex regional pain syndrome.
84. The method of any one of embodiments 1-83, wherein the method further comprises discontinuing the administration if the subject experiences an adverse event.
85. The method of any one of embodiments 1-84, wherein the psychedelic is administered with at least one additional therapeutic agent.
86. The method of any one of embodiments 1-85, wherein the method results in the induction of dissociative state in at least 70%, 80%, 90% or 95% of a plurality of subjects that have been administered the psychedelic.
87. The method of any one of embodiments 1-86, wherein the method results the maintenance of the mean plasma level of the psychedelic in at least 70%, 80%, 90% or 95% of a plurality of subjects that have been administered the psychedelic.
88. The method of any one of embodiments 1-87, wherein the subject is a human.
89. A method of treating a psychological disorder in a subject comprising
administering to the subject an amount of psilocybin or psilocin sufficient to induce a dissociative state in the subject less than 30 minutes after administration; and
thereafter maintaining the mean plasma concentration of the psychedelic at a predetermined value to maintain the dissociative state during a therapeutic window.
90. The method of embodiment 89, wherein the dissociative state is induced within 1, 2, 3, 4, 5, 6, 7, 8, 9, 10, 11, 12, 13, 14, 15, 16, 17, 18, 19, or 20 minutes.
91. The method of embodiment 89 or 90, wherein the therapeutic window is 0.5, 1, 2, 3, 4, 5, or 6 hours.
92. The method of any one of embodiments 89-91, wherein the Cₘₐₓ of the psychedelic occurs within 1-10 minutes of the administration.
93. The method of embodiment 92, wherein the Cₘₐₓ is about 1 μg/L-50 μg/L.
94. The method of any one of embodiments 89-93, wherein the dissociative state is induced by intravenous administration of an initial bolus of psychedelic at a dose of about 0.1 mg/kg to about 0.2 mg/kg.
95. The method of any one of embodiments 89-94, wherein the mean plasma concentration of the psychedelic is maintained at the predetermined value during the therapeutic window by continuous or intermittent administration of the psychedelic.
96. The method of embodiment 95, wherein the continuous or intermittent administration is via an intravenous route.
97. The method of embodiment 95, wherein the intermittent administration is via a subcutaneous, oral, transdermal, intramuscular, intranasal, intranasal/pharanygeal, or buccal route.
98. The method of embodiment 95 or 96, wherein the psychedelic is administered continuously at a rate of 0.1 mg/min to 1 mg/min.
99. The method of any one of embodiments 89-98, wherein the predetermined plasma concentration of the psychedelic is from 1-50 μg/L.
100. The method of any one of embodiments 89-99, wherein the method further comprises providing psychological support to the subject during the therapeutic window.
101. The method of any one of embodiments 89-100, further comprising withdrawing the subject from the dissociative state at the end of the treatment window by administering a 5HT_{2A} receptor antagonist.
102. The method of any one of embodiments 89-101, wherein the psychological disorder is selected from the group consisting of PTSD, alcohol addition, drug addiction, treatment resistant depression, anxiety, end of life anxiety, an eating disorder, fibromyalgia, neuropathic pain, phantom limb pain hypothalamic induced obesity and Prader-Willi syndrome, binge-eating.
103. The method of embodiment 102, wherein the psychological disorder is hypothalamic induced obesity, Prader-Willi syndrome, binge-eating or fibromyalgia.
104. The method of any one of embodiments 89-103, wherein the psychedelic is psilocybin, a co-crystal, co-former, or salt thereof, psilocin or a combination thereof.
105. The method of embodiment 104, wherein the psychedelic is psilocin, a co-crystal, co-former, or salt thereof.
106. The method of any one of embodiments 89-105, wherein the psychedelic is administered with at least one additional therapeutic agent.
107. A method of determining a therapeutically effective dose of a psychedelic for treating a psychological disorder in a subject, the method comprising;
administering to the subject an amount of psilocybin or psilocin sufficient to induce a dissociative state in the subject less than 30 minutes after administration; and
obtaining an EEG from the subject to determine when the subject enters the dissociative state
thereafter maintaining the mean plasma concentration of the psychedelic at a predetermined value to maintain the dissociative state during a therapeutic window.
108. The method of embodiment 107, further comprising obtaining a further EEG from the subject to monitor the dissociative state during the therapeutic window.
109. A psychedelic that is psilocybin, psilocin, a co-crystal, a co-former, or a salt thereof, or a combination thereof, for use in a method of any one of embodiments 1-108.
110. A psychedelic that is psilocybin, psilocin, a co-crystal, a co-former, or a salt thereof, or a combination thereof, for use in a method of treating a psychological disorder, wherein the method comprises the method of any one of embodiments 1-108.
111. A psychedelic that is psilocybin, psilocin, a co-crystal, a co-former, or a salt thereof, or a combination thereof, for use in a method of inducing a dissociative state, wherein the method comprises the method of any one of embodiments 1-108.
112. A psychedelic that is psilocybin, psilocin, a co-crystal, a co-former, or a salt thereof, or a combination thereof, for use in a method of determining a therapeutically effective dose of a psychedelic, wherein the method comprises the method of any one of embodiments 1-108.
113. A psychedelic that is psilocybin, psilocin, a co-crystal, a co-former, or a salt thereof, or a combination thereof, for use in a method of maintaining a dissociative state in a subject with a psychological disorder, wherein the method comprises the method of any one of embodiments 1-108.
114. A psychedelic that is psilocybin, psilocin, a co-crystal, a co-former, or a salt thereof, or a combination thereof, for use in a method of treating phantom limb pain.
115. A psychedelic that is psilocybin, psilocin, a co-crystal, a co-former, or a salt thereof, or a combination thereof, for use in a method of treating phantom limb pain, wherein the method comprises the method of any one of embodiments 1-108.
116. Use of a psychedelic that is psilocybin, psilocin, a co-crystal, a co-former, or a salt thereof, or a combination thereof, in the manufacture of a medicament for use in the method of any one of embodiments 1-108.
117. Use of a psychedelic that is psilocybin, psilocin, a co-crystal, a co-former, or a salt thereof, or a combination thereof, in the manufacture of a medicament for treating a psychological disorder in accordance with the method of any one of embodiments 1-108.
118. Use of a psychedelic that is psilocybin, psilocin, a co-crystal, a co-former, or a salt thereof, or a combination thereof, in the manufacture of a medicament for inducing a dissociative state in accordance with the method of any one of embodiments 1-108.
119. Use of a psychedelic that is psilocybin, psilocin, a co-crystal, a co-former, or a salt thereof, or a combination thereof, in the manufacture of a medicament for determining a therapeutically effective dose of a psychedelic in accordance with the method of any one of embodiments 1-108.
120. Use of a psychedelic that is psilocybin, psilocin, a co-crystal, a co-former, or a salt thereof, or a combination thereof, in the manufacture of a medicament for maintaining a dissociative state in a subject with a psychological disorder in accordance with the method of any one of embodiments 1-108.
121. Use of a psychedelic that is psilocybin, psilocin, a co-crystal, a co-former, or a salt thereof, or a combination thereof, in the manufacture of a medicament for treating phantom limb pain.
122. Use of a psychedelic that is psilocybin, psilocin, a co-crystal, a co-former, or a salt thereof, or a combination thereof, in the manufacture of a medicament for treating phantom limb pain in accordance with the method of any one of embodiments 1-108.
123. Use of a psychedelic that is psilocybin, psilocin, a co-crystal, a co-former, or a salt thereof, or a combination thereof, in the method of any one of embodiments 1-108.
124. Use of a psychedelic that is psilocybin, psilocin, a co-crystal, a co-former, or a salt thereof, or a combination thereof, for treating a psychological disorder in accordance with the method of any one of embodiments 1-108.
125. Use of a psychedelic that is psilocybin, psilocin, a co-crystal, a co-former, or a salt thereof, or a combination thereof, for inducing a dissociative state in accordance with the method of any one of embodiments 1-108.
126. Use of a psychedelic that is psilocybin, psilocin, a co-crystal, a co-former, or a salt thereof, or a combination thereof, for determining a therapeutically effective dose of a psychedelic in accordance with the method of any one of embodiments 1-108.
127. Use of a psychedelic that is psilocybin, psilocin, a co-crystal, a co-former, or a salt thereof, or a combination thereof, for maintaining a dissociative state in a subject with a psychological disorder in accordance with the method of any one of embodiments 1-108.
128. Use of a psychedelic that is psilocybin, psilocin, a co-crystal, a co-former, or a salt thereof, or a combination thereof, for treating phantom limb pain.
129. Use of a psychedelic that is psilocybin, psilocin, a co-crystal, a co-former, or a salt thereof, or a combination thereof, for treating phantom limb pain in accordance with the method of any one of embodiments 1-108.

### VII. EXAMPLES

The following examples are included for illustrative purposes only and are not intended to limit the scope of the invention.

### Example 1: Pharmacokinetics and Pharmacodynamics of Intravenous Infusion of Psychedelics in Rats

This example describes the pharmacokinetics and pharmacodynamics response in animals following intravenous infusion of psychedelics over a period of time. The plasma concentration of psilocin following a one-hour intravenous infusion of psilocybin or psilocin, and an indicator of the dissociative or psychedelic state, as determined by recorded head twitching movements, were assessed during the study. in.

Three male Sprague Dawley rats were administered a 10 mg/kg intravenous (IV) infusion over a one-hour (60 minutes) period with either psilocin or psilocybin, as described in Table E1. The psilocybin or psilocin was administered into a femoral vein catheter via an IV infusion set attached to a syringe pump over an interval of 60-minutes.

Pharmacokinetic parameters, including the plasma psilocin concentration, the maximum plasma concentration (Cₘₐₓ) and pharmacodynamics parameters, including incidences of head twitching behavior, as an indicator of a dissociative state, were assessed over time. All animals were between 7 and 12 weeks of age and 250 to 400 grams in weight with acceptable health assessments prior to the study. Prior to study, all animals were acclimated to the laboratory environment for one day. Animals were observed daily for signs of clinical changes.

TABLE E1
Intravenous infusion of administration
of psilocin or psilocybin in rats

| | | Dose | Dose | Dose |
|---|---|---|---|---|
| # Animals/ | Route of | Cone. | Volume | Level |
| Sex | Administration | (mg/mL) | (mL/kg) | (mg/kg) |
| 3M | IV (infusion) | 0.5 | 20 | 10 |

### A. Pharmacokinetics

Following initiation of the infusion, serial blood samples of about 0.5 mL each, were collected from each animal via the jugular vein catheter at several time points, including 0.25 hour, 0.5 hour, 1 hour, 2 hours, 4 hours, 6 hours and 8 hours from initiation of the infusion. Just before each blood sampling time point, a volume of blood slightly larger than the catheter tubing void volume was withdrawn and discarded. A 0.5 mL blood sample was obtained from the jugular vein catheter using a syringe with a blunted needle. The blood sample was immediately transferred into a chilled blood collection tube containing an anticoagulant and chilled. Chilled blood samples were centrifuged at 3000 RPM for 10 minutes at 4° C. to harvest plasma. Plasma samples were stored at −70° C. until ready for analysis.

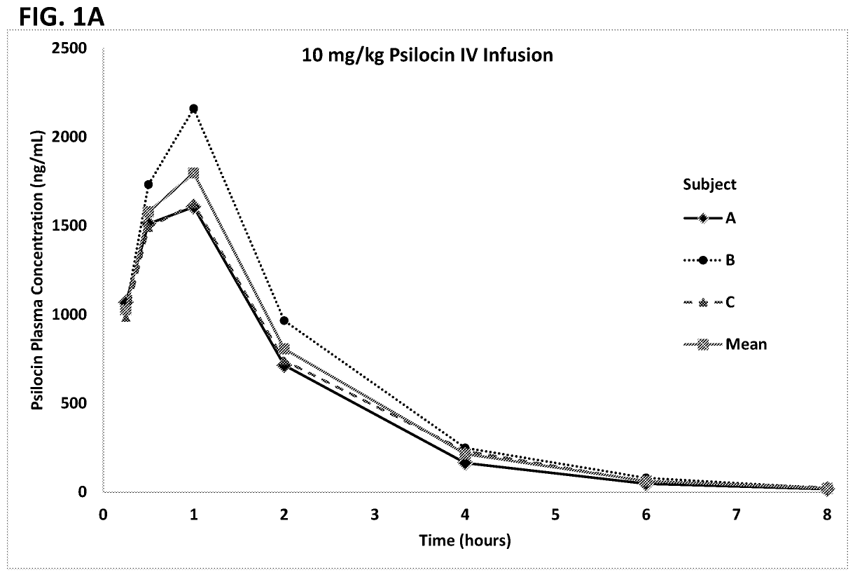

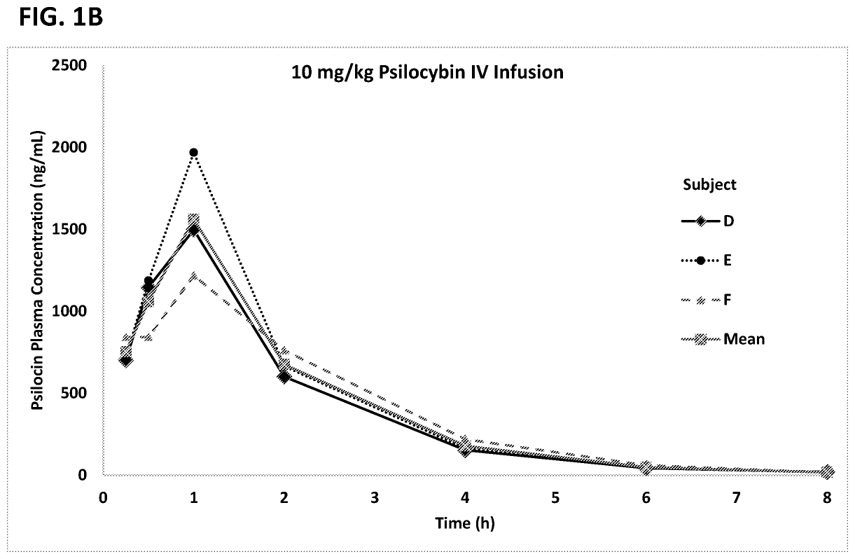

The plasma psilocin level as determined at each time point for each of the 3 rats that received IV psilocin infusion (Table E2; FIG. 1A) or IV psilocybin infusion (Table E4; FIG. 1B), and pharmacokinetic parameters, including the maximum plasma concentration (Cₘₐₓ), time of maximum plasma concentration (Tₘₐₓ), and the area under the curve at Tₘₐₓ (AUC_{T}), calculated based on the equation below (included assumption that concentration (ng/mL)=0 at time T=0): AUC_{T}=Σ₂^{N}^{ₘₐₓ}┌(Cₙ₋₁+Cₙ/2)×(Tₙ-Tₙ₋₁)┐, were determined. As shown, the mean plasma concentration at each time point and the Cₘₐₓ were remarkably similar in each animal, with small standard deviations, in both psilocin-(Tables E2 and E3) and psilocybin-treated animals (Tables E4 and E5). As shown in Tables E3 and E5, the AUC_{T} also were remarkably similar between the different animals. The results also showed consistent rapid reduction of the plasma concentration of the drug after the one-hour infusion is completed (see, reduction of plasma psilocin concentration at hours 2, 4, 6, and 8 in FIG. 1A and FIG. 1B).

The results support consistent plasma concentrations and exposure in different individual animal subjects during intravenous infusion of psilocybin or psilocin, and that the plasma concentration decreases rapidly and reproducibly after the completion of the intravenous infusion.

TABLE E2
Plasma concentration of psilocin (ng/mL) during
and following one-hour IV infusion of psilocin

| | Sampling Time (hours) | | | | | | |
|---|---|---|---|---|---|---|---|
| Subj. | 0.25 | 0.5 | 1 | 2 | 4 | 6 | 8 |
| A | 1067.0 | 1510.9 | 1606.1 | 714.5 | 164.5 | 47.7 | 17.3 |
| B | 1031.8 | 1731.6 | 2159.5 | 965.6 | 249.1 | 80.2 | 22.5 |
| C | 982.7 | 1489.3 | 1623.2 | 740.4 | 231.7 | 59.6 | 26.8 |
| MEAN | 1027.2 | 1577.3 | 1796.3 | 806.8 | 215.1 | 62.5 | 22.2 |
| S.D. | 42.3 | 134.1 | 314.7 | 138.1 | 44.7 | 16.4 | 4.8 |
| C.V. | 4.1% | 8.5% | 17.5% | 17.1% | 20.8% | 26.3% | 21.4% |
| N | 3 | 3 | 3 | 3 | 3 | 3 | 3 |

TABLE E3
Pharmacokinetic parameters following psilocin IV infusion

| | Cₘₐₓ | Tₘₐₓ | AUC_{T} |
|---|---|---|---|
| Subj. | (ng/mL) | (h) | (ng · h/mL) |
| A | 1606.1 | 1 | 3551.4 |
| B | 2159.5 | 1 | 4656.4 |
| C | 1623.2 | 1 | 3741.6 |
| MEAN | 1796.3 | 1 | 3983.1 |
| S.D. | 314.7 | 0 | 590.8 |
| C.V. | 17.5% | 0% | 14.8% |
| N | 3 | 3 | 3 |

TABLE E4
Plasma concentration of psilocin (ng/mL) during
and following one-hour IV infusion of psilocybin

| | Sampling Time (hours) | | | | | | |
|---|---|---|---|---|---|---|---|
| Subj. | 0.25 | 0.5 | 1 | 2 | 4 | 6 | 8 |
| D | 701.6 | 1146.6 | 1497.3 | 600.6 | 153 | 43 | 19.4 |
| E | 715.6 | 1186.6 | 1968.6 | 664.1 | 162.8 | 42.2 | 16.7 |
| F | 843 | 842.9 | 1218.3 | 761.9 | 219.4 | 59.8 | 16.3 |
| MEAN | 753.4 | 1058.7 | 1561.4 | 675.5 | 178.4 | 48.3 | 17.5 |
| S.D. | 77.9 | 188.0 | 379.2 | 81.3 | 35.8 | 9.9 | 1.7 |
| C.V. | 10.3% | 17.8% | 24.3% | 12.0% | 20.1% | 20.6% | 9.7% |
| N | 3 | 3 | 3 | 3 | 3 | 3 | 3 |

TABLE E5
Pharmacokinetic parameters following psilocybin IV infusion

| | Cₘₐₓ | Tₘₐₓ | AUC_{T} |
|---|---|---|---|
| Subj. | (ng/mL) | (h) | (ng · h/mL) |
| D | 1497.3 | 1 | 3040.7 |
| E | 1968.6 | 1 | 3523.2 |
| F | 1218.3 | 1 | 3158.1 |
| MEAN | 1561.4 | 1 | 3240.6 |
| S.D. | 379.2 | 0 | 251.6 |
| C.V. | 24.3% | 0% | 7.8% |
| N | 3 | 3 | 3 |

### B. Pharmacodynamics/Behavior

To assess the pharmacodynamics (PD) of intravenously infused psychedelics, animal head-twitching was recorded as an indicator of achieving the dissociative state. Head twitching response (HTR) is used as a surrogate PD marker for the animals achieving or being in the dissociative or psychedelic state (for example, as described in Zhuk et al., Toxins 2015, 7, 1018-1029; Halberstadt et al., J Psychopharmacol. 2011 25(11): 1548-1561). HTR was also used to determine when the end of the dissociative state occurred following discontinuation of the IV infusion.

To assess HTR, a video monitoring system was used to record the animals in this study from discontinuation of the infusion through to the terminal sampling time point (1-hour post infusion). A camera was positioned such that the animals were visible from above. The recording was saved for review and the time of the observed head-twitching behavior, the duration of the observed behavior, and any additional atypical observations were documented.

The number of head-twitching events observed within the 60 minute time period after the one hour intravenous infusion of each of the 3 rats receiving psilocin or psilocybin, were determined from the video recordings. Head-twitching was observed in 2 of 3 rats receiving psilocin IV infusion, and 2 of 3 rats receiving psilocybin IV infusion, each animal experiencing 2-6 head-twitching events during the 60 minute time period after the one hour intravenous infusion. Notably, for rats receiving psilocin, no head twitching was observed in any animals more than 16 minute after the termination of infusion, correlating with rapid and controlled reversion from the psychedelic or dissociative state. For rats receiving psilocybin, head twitching was observed for up to 50 minutes after the termination of infusion. Upon termination of the IV infusion of either psilocin or psilocybin, head twitching activity subsided within 50 minutes, supporting the rapid termination of the dissociative or psychedelic, compared to previously reported termination of the dissociative state after an oral administration of psilocybin.

Oral route of administration of psychedelics provides no mechanism for control of blood levels, and is not amenable to control of the duration of the dissociative state or the psychedelic experience, or the ability to terminate or reverse adverse events. Following a 25 mg oral capsule the onset of the dissociative of psychedelic state commences between 1-2 hours post administration, and can last for 6-8 hours with no ability to manipulate or terminate the experience (see, e.g., Hasler et al., (1997) Pharm Acta Helv, 72(3), 175-184). Intravenous administration allows the duration of the dissociative state or the psychedelic experience be controlled and in case of an adverse event, the experience terminated, as evidenced by the rapid decrease in blood levels and pharmacodynamic markers of the dissociative or psychedelic state.

The results demonstrated that intravenous infusion of psychedelics (psilocin or psilocybin) over a period of time, such as one hour, results in rapid achievement of effective concentrations for inducing a dissociative state, consistent and reproducible plasma levels, and a further consistent and reproducible decrease in plasma levels after termination of the infusion. This is in contrast to previous reports plasma levels after oral administration, which were shown to result in wide variability among subjects (see, e.g., Brown et al. (2017). Clin Pharmacokinet. 56(12): 1543-1554). Intravenous administration, as demonstrated herein, provides a method of administration that allows for more rapid controlled entrance into the dissociative state depending on the rate of administration, the ability to control the duration of time in the dissociative state based upon the duration of the administration, and provide for a more rapid termination of the dissociative state should adverse events occur. The results also support the ability to monitor and maintain mean plasma concentration of the psychedelic, such as the active metabolite psilocin, during a therapeutic window.

The results support the utility of intravenous infusions as a method for administration for achieving consistent and controllable plasma concentration of psilocin (the active pharmacologic agent). The results also support the utility of monitoring plasma levels of psilocin, as a means to assess and correlate with a dissociative state with a target plasma level of psilocin, and monitoring the maintenance of mean plasma concentration of the psychedelic at a specific value during a therapeutic window. The results support the utility of intravenous administrations, including intravenous infusions, and monitoring of behaviors, for example head-twitching behaviors in rats, which can be generally translated to a dissociative state or psychedelic effects in humans, as an exemplary non-invasive measurement of brain activity during or after administration, to monitor the entry into and exit from the dissociative state and pharmacodynamics of the administered psychedelic.

### Example 2: Monitoring of Dissociative State Before, During, and After Intravenous Infusion of Psychedelics using Electroencephalography (EEG)

This example describes another exemplary non-invasive measurement of brain activity using electroencephalograph (EEG), in animals that have received an intravenous infusion of a psychedelic over a period of time.

The changes in neurophysiological complexity and brain response to intravenous infusion of psilocybin or psilocin was assessed in rats, before, during and after a 10 mg/kg intravenous (IV) infusion over 60 minute period with either psilocybin or psilocin. Sprague Dawley rats were subject to 60 minute IV infusions with psilocybin or psilocin, generally as described in Example 1. The changes in EEG were monitored before, during and after the infusion. In addition, incidences of head twitching response was assessed before, during and after the infusion, as an additional indicator of a pharmacodynamics response.

To prepare the animals for IV drug infusion and EEG monitoring, the rats were first anesthetized according to standard procedures to perform the surgery for IV cannulation. An indwelling cannula was inserted into the jugular vein and externalized at the base of the skull for IV infusion of psilocin or psilocybin. The externalized canula was attached to an infusion port that allowed IV infusion to freely moving and conscious animals.

The rats were anesthetized and positioned in the stereotaxic equipment (Model 963, David Kopf Instruments, Tujunga, CA) for EEG electrode implantation. The stereotaxic equipment allowed for 3D positioning of the electrodes in precise regions of the rat's brain. The cranium was exposed, and burr holes (30) were drilled to implant stainless-steel screw electrodes (B000FN89DM, Small Parts, Logansport, IN) for recording EEG from across the cortex. A screw electrode was implanted over the nasal sinus to serve as the reference electrode, and another screw electrode was implanted over the cerebellum as a ground electrode. The electrodes were interfaced with a pedestal and the entire assembly along with the infusion port for venous catheter infusions was secured with dental acrylic (methyl methacrylate).

For EEG recording and data acquisition, monopolar EEG data from each rat was acquired using a 32-channel Cereplex μ headstage (Blackrock Microsystems, Salt Lake City, UT). The signals were digitized at 1 kHz and bandpass filtered between 0.1-1 kHz using a Cereplex Direct system paired with the Cereplex Direct Software.

Prior to drug IV administration, baseline EEG was recorded for 20 minutes under freely moving, normally behaving conditions. Immediately after baseline recording, continuous intravenous infusion of psilocybin (10 mg/kg at the rate of 5 mL/kg/h) was initiated. The infusion lasted for 60 minutes during which EEG data were recorded. Post-infusion EEG data were also collected for a period for 120 minutes for each animal.

Once EEG results were collected, the raw EEG signals were exported into MATLAB (version 2021a; MathWorks, Inc., Natick, MA), and down-sampled to 500 Hz. Seven 5-minute artifact-free EEG epochs were extracted from the 1) baseline period, 2) beginning, middle, and end of the psilocybin or psilocin infusion period, and 3) the post-psilocybin/psilocin infusion recovery period. Prior to the analysis, the signal was detrended using a local linear regression method with a 10-second window at a 5-second step size in Chronux analysis software (Mitra & Bokil, Observed Brain Dynamics; Oxford University Press, NY, 2008). Line noise (60 Hz) and its harmonics were identified and removed via a multi-taper regression approach while minimizing background signal distortion using CleanLine EEGLAB plugin (Mullen T., Neuroimaging Informatics Tools Resource, Clear, San Diego: 2012). The signals were lowpass filtered at 30 Hz via Butterworth filter of order 5 and divided into non-overlapped 2-second windows for analysis.

Lempel-Ziv complexity (LZC)—a surrogate for neural signal diversity—was used to analyze EEG complexity before, during, and after psilocybin infusion (Lempel and Ziv, IEEE Trans Inform Theory 1976; 22(1):75-81). The LZC analysis was performed as previously described (Schartner et al., PLOS One 2015; 10(8):e0133532; Schartner et al., Sci Rep 2017; 7:46421; Schartner et al., Neuroscie Conscious 2017; 2017(1):niw022; Li and Mashour, Neuroimage 2019; 196:32-40; Pal et al., J Neurosci 2020; 40(3):605-18).

After pre-processing, for each 2-second window, the instantaneous amplitude was calculated from the Hilbert transform of the signal, which was then binarized using its mean value as the threshold for each channel. The LZC values were then averaged over each 5-minute epoch. The original LZC was normalized by using the mean of the LZC values from n=50 surrogate time series generated by randomly shuffling the binary sequence, as this leads to maximal value for binary sequences for a fixed length.

To examine if the complexity as reflected by the LZC is dissociable from the spectral content of the signal, an additional control measure was applied that compared the time-shuffled LZC (as described above) with LZC normalized through phase-randomization, which was obtained from surrogate data in which the power spectrum of the signal was preserved. The resultant normalized LZC measure (denoted LZC_{N}) reflected changes in complexity beyond the spectral content of the signal. Both LZC and LZC_{N} were reported in arbitrary units. LZC and LZC_{N} for each channel were computed separately and then averaged across channels.

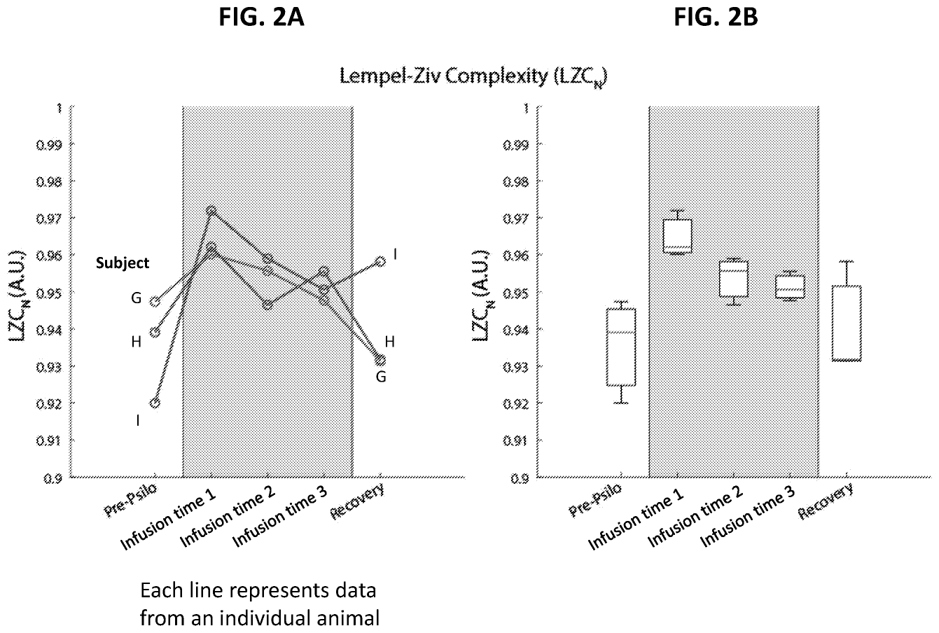

As shown in FIG. 2A and FIG. 2B, for animals receiving a 60 minute psilocybin infusion, temporal LZC_{N} estimations indicated an increase in 1-30 Hz signal complexity during the 5 minutes after the infusion began, which was sustained for the duration of the infusion. Following the end of the infusion period, temporal LZC_{N} rapidly returned to pre-infusion baseline levels. These changes were observed consistently across three rats (FIG. 2A: individual rats G, H and I; FIG. 2B; mean). These results are consistent with the observations of temporal LZC_{N} in human magnetoencephalography (MEG) data, indicating that psilocybin infusion has corresponding impacts on brain network dynamics in humans and rats.

In a separate study, unrestrained rats were infused with 10 mg/kg psilocybin over a 60 minute period and head twitching response (HTR) was recorded prior to during and after IV administration. HTR was consistently observed in all tested rats, within 5 minutes after initiation of the infusion, and persisted during the 60-minute infusion period. HTR was discontinued in the animals soon after the termination of the infusion, generally within 30 minutes after the termination of the infusion. The results of the HTR supported the more rapid achievement of the dissociative or psychedelic state, coincident with the rise in systemic plasma psilocin levels, and rapid exit from the dissociative or psychedelic state after the termination of infusion (e.g., as assessed in Example 1).

The results demonstrated that IV administration of psychedelics, such as psilocybin or psilocin, results in a rapid entrance into the dissociative state, which can be maintained during a continuous infusion period, as evidence by changes on EEG measurements. Further, upon termination of the IV infusion, the subjects rapidly returned from the dissociative state to baseline EEG measurements. The results showed that IV administration of psychedelics can achieve a rapid entrance into, maintenance of, and exit from the dissociative state, as shown by EEG, an exemplary non-invasive measurement of brain activity. In addition, the results of the head twitch response (HTR) measurements and the pharmacokinetic parameters, also supported the rapid entrance, maintenance and exit from the dissociative state.

The results support the utility of administering psychedelics by intravenous administration, including an intravenous infusion, which could be used to rapidly and accurately control the dissociative state in a subject, in addition to consistent, reproducible and controllable pharmacokinetics and pharmacodynamics, in contrast with oral administration, which is shown to result in wide variability among subjects. The results also support the utility of using methods of non-invasive measurement of brain activity, such as EEG, for monitoring the dissociative state, determining therapeutically effective doses, and assessing, adjusting and tailoring the therapeutic treatment depending on the subject's response.

### Example 3: Pharmacokinetics after Intravenous Bolus Administration of Psychedelics

The plasma psilocin concentrations over time after an intravenous bolus administration of psilocybin was assessed.

Rats that have received isoflurane anesthetic (ISO+) or that have not received isoflurane anesthetic (ISO−) were administered an intravenous bolus of 10 mg/kg psilocybin, administered over 3 minutes. Plasma psilocin concentrations were measured, generally as described in Example 2 above. Blood samples were periodically collected over approximately 40 minutes after the 3 minute bolus administration.

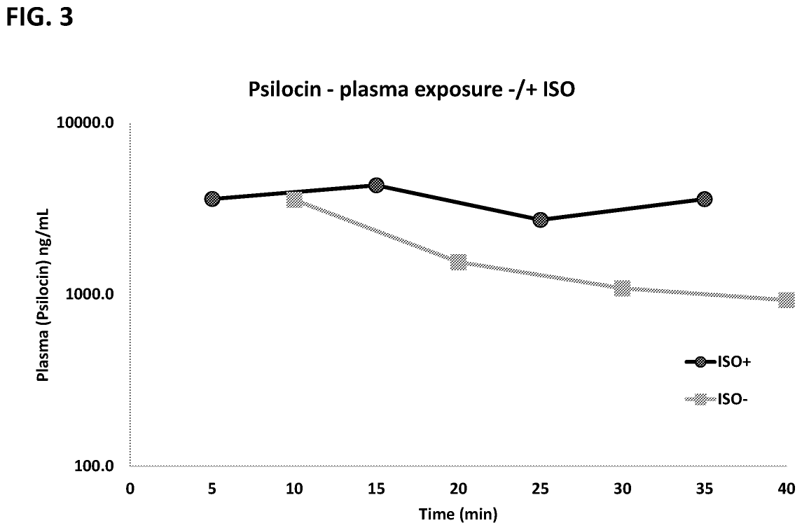

Plasma psilocin levels after the psilocybin bolus infusion, in animals that have received isoflurane anesthetic (ISO+) or that have not received isoflurane anesthetic (ISO−) are shown in FIG. 3. The results show a steady plasma concentration for at least 40 minutes after the intravenous bolus administration and at blood levels sufficient to achieve the psychedelic state.

### Example 4: Effect of Intravenous Administration of Psychedelics on Diseases Associated with Pain

This example describes the effects of intravenous administration of psychedelics on pain perception and diseases associated with pain, such as nociplastic pain, in an animal model of pain.

Chronic pain in rats was induced by injection of formalin into the hind paw. The rats subsequently were intravenously administered psilocybin, and assessed for changes in pain perception over an extended period of time. The formalin pain model is a translational animal model for chronic nociplastic pain (Vanini G., Sleep 2016; 39(1): 133-42), including fibromyalgia, phantom limb pain or complex regional pain syndrome. In the formalin pain model, in addition to experiencing extra pain in the formalin-injected paw, the animal experiences increased pain perception (i.e., hyperalgesia) in extremities not injected with formalin (e.g., the contra-lateral hind paw compared to the one injected with formalin). The pain perception was measured in the contra-lateral paw to assess the potential benefit of psychedelics in nociplastic pain disorders. Reduction in pain perception and the duration of pain reduction were examined following intravenous bolus administration of 10 mg/kg psilocybin, administered over 3 minutes.

Two types of pain perception were measured. First, the Von Frey assay (VFA) was used to measure pain due to pressure exerted on the extremity (i.e., mechanical allodynia). Specifically. VFA employed seven filaments: 1, 2, 4, 6, 8, 10 and 15 g. Each filament was applied in ascending order to the plantar surface of the rat's hind paw until bent. Upon bending the filament, the filament was held in place for 5 seconds or until the rat withdrew its paw. The stimulus was not applied if the rat was walking, grooming, or sniffing. The stimulus was applied to the injected and contra-lateral hind paws for five trials each, with at least 30 second breaks between stimuli. The minimum amount of force required to evoke a paw withdrawal response in each trial was determined to be the minimum withdrawal threshold for that animal trial. After five trials for each paw, the minimum amount of force required to evoke a paw withdrawal response was averaged across all trials to calculate the average minimum withdrawal threshold for each paw, respectively.

The second pain measurement was based on the Hot Plate Assay (HPA), which measures the withdrawal latency at temperatures that register a painful response (i.e., thermal hyperalgesia). The HPA was initiated 15-20 minutes after completion of the VFA. A hot plate was preheated to a temperature of 52.5° C. Once heated, each animal was placed in cylindrical tube on the hot plate and the latency to lick a hind paw or observed overt escape behavior (e.g., attempt to jump out of cylinder) was recorded (Ingram et al., Neruopsychopharmacology 2007; 32(3):600-6; Gunn et al., J Pain 2011; 12(2):222-7; Hestehave et al., Lab Anim 2016; 51(3):264-72). For safety purposes, the maximum time for a rat to be on hot plate was limited to 30 seconds. After a 15 minute break, this was exercise was repeated once more.

The full examination of pain perception lasted for 30 days for each animal, as described Table E6 below.

TABLE E6
Testing Procedure and Schedule

| Day(s) | Procedures | |
|---|---|---|
| Day −1 | I. | Test VFA withdrawal threshold baseline at 2 PM (+/−1 hour) |
| | II. | If baseline VFA showed abnormal results (<10 g), the animal was |
| | | reevaluated. Rats that show persistent, reduced baseline levels |
| | | (<10 g) were excluded from the study |
| | III. | Test HPA withdrawal latency baseline at 3 PM (+/−1 hour) |
| Day 0 | I. | Administer subcutaneous 50 μl injection of 5% formalin on the |
| | | ventral surface of right hind paw (around 12 PM). Right and left hind |
| | | paw were counterbalanced for each experimental cohort (i.e., some |
| | | rats received formalin injection in the left paw, others in the right |
| | | paw) |
| | II. | Measure VFA withdrawal threshold (1-3 PM) |
| | III. | Measure HPA withdrawal latency (2-4 PM) |
| Day 2 | I. | Administer 10 mg/kg intravenous psilocybin in a 0.9% saline, or |
| | | saline only control, over 3 minutes (11 AM) |
| | II. | Measure VFA withdrawal threshold (1-3 PM) |
| | III. | Measure HPA withdrawal latency (2-4 PM) |
| Days 4, 6, 8, | I. | Measure VFA withdrawal threshold (1-3 PM) |
| 15, 22, and 29 | II. | Measure HPA withdrawal latency (2-4 PM) |

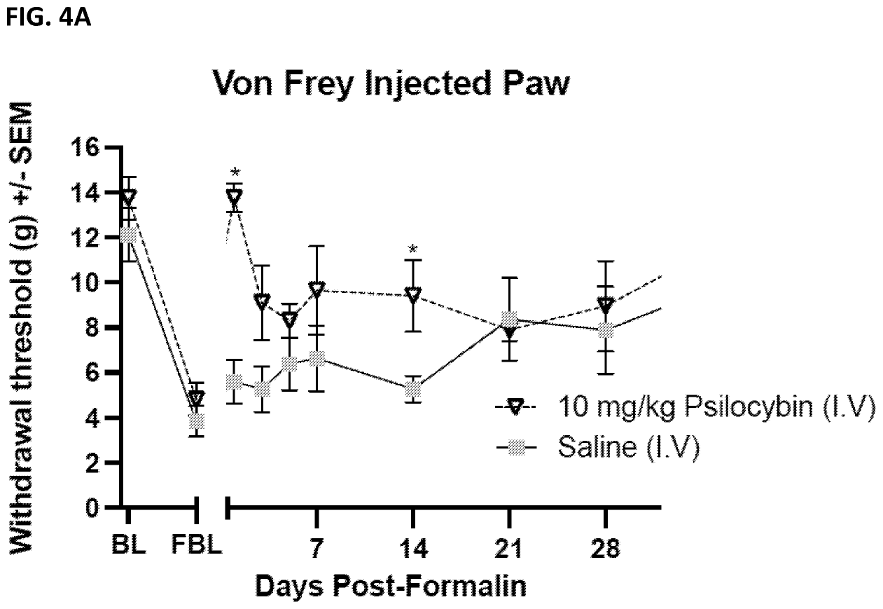

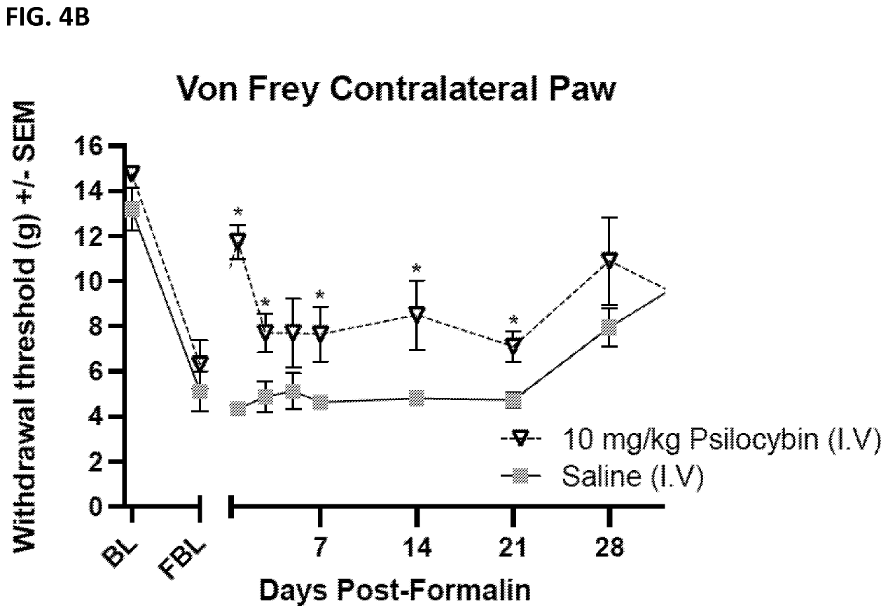

FIG. 4A and FIG. 4B show the results for the VFA, comparing the effects of psilocybin or saline injection on withdrawal threshold (i.e., mechanical allodynia). The withdrawal threshold for both groups was initially high at baseline (BL) and substantially reduced after formalin injection (FBL). After psilocybin or saline injection on day 2, a marked and acute difference in withdrawal threshold was observed in both the formalin-injected paw (FIG. 4A) and the contra-lateral paw (FIG. 4B), indicative of analgesic properties of psilocybin administration. For other analgesic drugs (e.g., NSAID, opioids), this response rapidly disappears after drug clearance from the circulation. Rats administered intravenous psilocybin showed reduced pain sensitivity for at least two weeks after a single psilocybin administration, for both the injected paw (FIG. 4A) and the contra-lateral paw (FIG. 4B). In the contra-lateral paw, a statistically significant increase in withdrawal threshold was observed for 3 weeks after a single psilocybin administration.

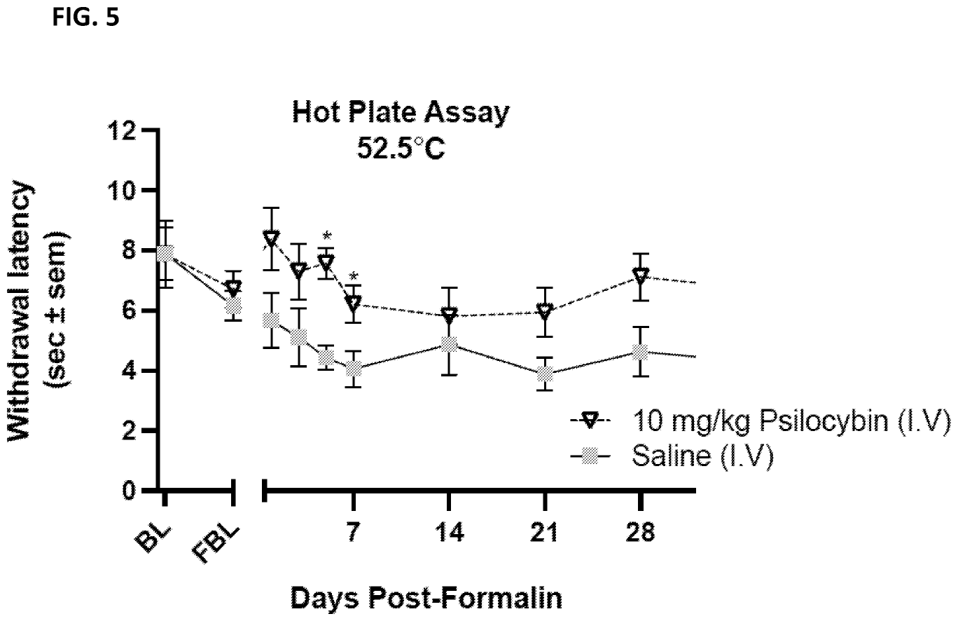

As shown in FIG. 5, similar results were observed for thermal sensitivity, as shown by the HPA results. After an acute recovery response in withdrawal latency after psilocybin administration, the improved pain sensitivity response was sustained for at least 4 weeks compared to saline control.

The results demonstrated acute and lasting effects of intravenous administration of psychedelics, such as psilocybin or psilocin, on mechanical and thermal pain sensitivity. The results showed long-lasting improvements on pain sensitivity, for at least up to 4 weeks in an animal model of nociplastic pain. Importantly, the effects were also observed on the non-injected contra-lateral paw, which is closely associated with nociplastic pain. The results support the utility of administering psychedelics by intravenous administration, for treating and improving diseases and disorders related to pain perception, including nociplastic pain disorders such as fibromyalgia, phantom limb pain or complex regional pain syndrome.

### Example 5: Intravenous Administration of Psilocin or Psilocybin and Monitoring in Human Subjects

Psychedelics such as psilocin and psilocybin are administered by intravenous administration to human subjects, such as human subjects with a psychological disorder or a disease with a psychological component. Entrance to and exit from the dissociative or psychedelic state and the plasma levels of the psychedelic, is monitored and assessed. In some aspects, the intravenous administration permits induction of a dissociative state in the subject less than 30 minutes after administration, and the plasma concentration of the psychedelic is maintained at a predetermined value to maintain the dissociative state during a therapeutic window.

Adult human subjects that are not concurrently taking any medications having a primary centrally-acting serotonergic effect (such as selective serotonin reuptake inhibitors (SSRIs) or monoamine oxidase inhibitors (MAOIs)) are administered one of the following doses of psychedelics: (1) 2.5 mg psilocin administered by intravenous infusion over 60 minutes; (2) 5 mg psilocybin administered by intravenous infusion over 60 minutes; (3) 25 mg psilocybin administered orally; (4) a total of 10 mg psilocin, administered as an loading dose at a rate of 1 ml/min infusion during the first 10 or 15 minutes of the infusion, and then adjusted to a maintenance dose at a rate of 0.5 ml/min infusion for the remaining time up to 120 minutes of total time of infusion; (5) a total of 10 mg psilocybin, administered as an loading dose at a rate of 1 ml/min infusion during the first 10 or 15 minutes of the infusion, and a maintenance dose at a rate of 0.5 ml/min infusion for the remaining time up to 120 minutes of total time of infusion. In most cases, the total amount of psilocybin or psilocin administered intravenously is up to about 10 mg per subject.

In some aspects, the initial dose, (i.e., loading dose) is sufficient to induce a dissociative state in the subject within 30 minutes of the initiation of administration. In some aspects, the maintenance dose is sufficient to maintain the mean plasma concentration of the psychedelic at a predetermined value to maintain the dissociative state during a therapeutic window.

The subject is monitored before, during and after the administration. The subject's vital signs, for example blood pressure (BP), pulse, and heart rate (HR), as well as the dissociative state, are monitored during and after the administration, for example, before administration and at one or more of 30, 60, 90, 120, 180, 240, 300, and 360 minutes after administration. Session monitors complete a Monitor Rating Scale (MRS) Form, which involves rating or scoring several dimensions of the subject's behavior and mood, e.g., presence/intensity of behaviors, signs, and reported symptoms, such as peacefulness, yawning, nausea/vomiting, quantity of speech, anxiety, sleepiness, crying, restlessness, visual changes, euphoria, and feelings of unreality.

In some aspects, blood samples are collected before administration, and at one or more of 5, 10, 15, 30, 45, 60, 90 minutes and, 2, 3, 4, 6, 8, 12 and 24 hours after the initiation of administration, and the plasma concentration of psilocybin and psilocin are determined at the time points. In some aspects, the subject is monitored with an exemplary non-invasive measurement of brain activity, such as by electroencephalograph (EEG). In cases of EEG, the subject is monitored before, during and after the infusion.

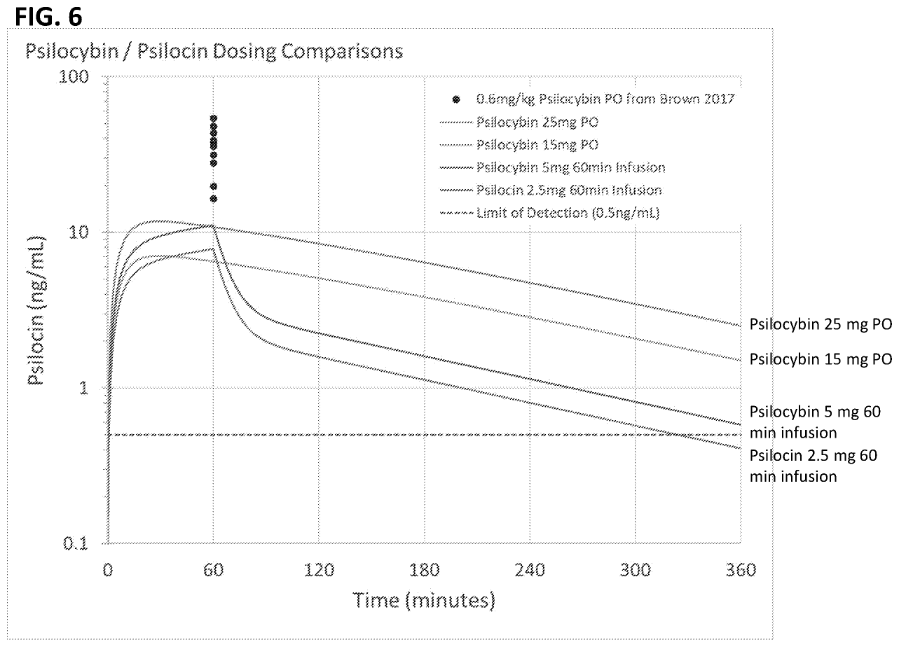

FIG. 6 shows an exemplary projected plasma psilocin concentration in human subjects over 360 minutes after administration of: 2.5 mg psilocin administered by intravenous infusion over 60 minutes; 5 mg psilocybin administered by intravenous infusion over 60 minutes; 15 mg oral psilocybin or 25 mg oral psilocybin. The plot also includes observed plasma concentration from oral psilocybin administration at 0.6 mg/kg as described in Brown et al. (2017). Clin Pharmacokinet. 56(12):1543-1554.

The subject is also monitored and evaluated for outcomes related to safety, entry and exit from a dissociative state, and treatment outcomes, for example, treatment of the psychological disorder or a disease with a psychological component.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

1. A method of treating a psychological disorder in a subject, the method comprising:
administering to a subject having a psychological disorder an amount of a psychedelic sufficient to induce a dissociative state in the subject less than 30 minutes after administration; and
thereafter maintaining the mean plasma concentration of the psychedelic at a predetermined value to maintain the dissociative state during a therapeutic window; wherein the psychedelic is psilocybin, psilocin, a co-crystal, a co-former, or a salt thereof, or a combination thereof.

2. The method of claim 1, wherein the method further comprises obtaining a non-invasive measurement of brain activity from the subject to determine when the subject enters the dissociative state.

3. A method of inducing a dissociative state, the method comprising:
administering to a subject having a psychological disorder, an amount of a psychedelic sufficient to induce the dissociative state in the subject; and
obtaining a non-invasive measurement of brain activity from the subject to determine when the subject enters the dissociative state; wherein the psychedelic is psilocybin, psilocin, a co-crystal, a co-former, or a salt thereof, or a combination thereof.

4. A method of inducing a dissociative state, the method comprising:
administering to a subject having a psychological disorder, an amount of a psychedelic sufficient to induce the dissociative state in the subject; and
obtaining a measurement of electroencephalography (EEG) from the subject to determine when the subject enters the dissociative state; wherein the psychedelic is psilocybin, psilocin, a co-crystal, a co-former, or a salt thereof, or a combination thereof.

5. A method of determining a therapeutically effective dose of a psychedelic, the method comprising:
administering to a subject having a psychological disorder, an amount of a psychedelic sufficient to induce a dissociative state in the subject;
obtaining a non-invasive measurement of brain activity from the subject to determine if the subject enters the dissociative state; and
determining the amount of the psychedelic as a therapeutically effective dose if the subject enters the dissociative state; wherein the psychedelic is psilocybin, psilocin, a co-crystal, a co-former, or a salt thereof, or a combination thereof.

6. A method of maintaining a dissociative state in a subject with a psychological disorder, the method comprising;
administering to the subject an amount of a psychedelic sufficient to induce the dissociative state in the subject less than 30 minutes after administration; and
obtaining a measurement of electroencephalography (EEG) from the subject to determine when the subject enters the dissociative state; and
thereafter maintaining a mean plasma concentration of the psychedelic at a predetermined value to maintain the dissociative state during a therapeutic window; wherein the psychedelic is psilocybin, psilocin, a co-crystal, a co-former, or a salt thereof, or a combination thereof.

7. A method of treating a psychological disorder, the method comprising:
performing the method of any one of claims 1-6; and
maintaining the mean plasma concentration of the psychedelic at a predetermined value to maintain the dissociative state during a therapeutic window; thereby treating the psychological disorder.

8. A method of treating phantom limb pain in a subject, the method comprising
administering to a subject having phantom limb pain an amount of a psychedelic sufficient to induce a dissociative state in the subject; wherein the psychedelic is psilocybin, psilocin, a co-crystal, a co-former, or a salt thereof, or a combination thereof.

9. The method of any one of claims 1-8, wherein the administration of the psychedelic is by intravenous administration.

10. The method of any one of claims 1-5 and 7-9, wherein the dissociative state is induced in the subject within less than at or about 15, 30, 60, 90 or 120 minutes after administration, or a range defined by any of the foregoing.

11. The method of any one of claims 1-5 and 7-10, wherein the dissociative state is induced in the subject within less than at or about 30 minutes after administration.

12. The method of any one of claims 3-5 and 8-11, wherein the method further comprises maintaining the mean plasma concentration of the psychedelic at a predetermined value to maintain the dissociative state during a therapeutic window.

13. The method of any one of claims 2, 3, 5, 7, and 9-12, wherein the non-invasive measurement of brain activity is selected from among electroencephalography (EEG), functional magnetic resonance imaging (fMRI), near-infrared spectroscopy (NIRS), magnetoencephalography (MEG), and optoencephalography (OEG).

14. The method of any one of claims 2, 3, 5, 7, and 9-13, wherein the non-invasive measurement of brain activity is EEG.

15. The method of any one of claims 2, 3, 5, 7, and 9-12, wherein the non-invasive measurement of brain activity is questionnaire-based evaluation of the experience.

16. The method of claim 15, wherein the questionnaire-based evaluation of the psychedelic experience is selected from among one or more of: the mystical experience questionnaire (MEQ30) evaluation, the challenging experience questionnaire (CEQ) evaluation, the psychological insight questionnaire (PIQ) evaluation, a qualitative written assessment, and/or the monitor rating scale (MRS) questionnaire.

17. The method of any one of claims 2, 3, 5, 7, and 9-16, wherein the method further comprises obtaining a further non-invasive measurement of brain activity from the subject to monitor the dissociative state during the therapeutic window.

18. The method of any one of claims 4, 6-12, and 14, wherein the method further comprises obtaining a further measurement of EEG from the subject to monitor the dissociative state during the therapeutic window.

19. The method of any one of claims 4, 6-12, 14, and 18, wherein the measurement of EEG is analyzed as expressed by Lempel-Ziv complexity (LZC).

20. The method of any one of claims 2, 3, 5, 7, and 9-17, wherein the non-invasive measurement of brain activity is obtained prior to, during and/or after administration.

21. The method of claim 20, wherein the non-invasive measurement of brain activity after administration indicates the termination of dissociative state.

22. The method of any one of claims 4, 6-12, 14, 18, and 19, wherein the measurement of EEG is obtained prior to, during and after administration.

23. The method of claim 22, wherein the measurement of EEG after administration indicates the termination of dissociative state.

24. The method of any one of claims 1-23, wherein the dissociative state is induced within at or about 1, 2, 3, 4, 5, 6, 7, 8, 9, 10, 11, 12, 13, 14, 15, 16, 17, 18, 19, or 20 minutes after the administration.

25. The method of any one of claims 1-24, wherein the dissociative state is induced within at or about 5 minutes after the administration.

26. The method of any one of claims 1, 6, 7, and 9-25, wherein the therapeutic window is at or about 0.5, 1, 2, 3, 4, 5, or 6 hours.

27. The method of any one of claims 1, 6, 7, and 9-26, wherein the therapeutic window is between at or about 0.5 hours and at or about 4 hours.

28. The method of any one of claims 1-27, further comprising terminating administration of the psychedelic to terminate the dissociative state.

29. The method of claim 28, wherein the termination of dissociative state occurs within at or about 30, 60, 90, 120, or 180 minutes after the termination of administration of the psychedelic, or a range defined by any of the foregoing.

30. The method of claim 28 or 29, wherein the termination of dissociative state occurs within at or about 60 minutes after the termination of administration of the psychedelic.

31. The method of any one of claims 1-30, wherein the psychedelic is administered to produce a predetermined Cₘₐₓ within at or about 30-120 minutes after the initiation of the administration.

32. The method of any one of claims 1-31, wherein the psychedelic is administered to produce a predetermined Cₘₐₓ within at or about 45-90 minutes after the initiation of the administration.

33. The method of any one of claims 1-30, wherein the psychedelic is administered to produce a predetermined Cₘₐₓ within at or about 1-10 minutes of the administration.

34. The method of any one of claims 30-33, wherein the Cₘₐₓ is at or about 1 μg/L-50 g/L.

35. The method of any one of claims 30-34, wherein the Cₘₐₓ is at or about 10 μg/L-20 μg/L.

36. The method of any one of claims 30-35, wherein the Cₘₐₓ is at or about 10 μg/L-15 μg/L.

37. The method of any one of claims 1-36, wherein the dissociative state is induced by intravenous administration of a loading dose of the psychedelic.

38. The method of claim 37, wherein the loading dose comprises administration of an initial bolus of the psychedelic.

39. The method of claim 37 or 38, wherein the initial bolus dose is at or about 1 mg, 2 mg, 3 mg, 4 mg or 5 mg.

40. The method of any one of claims 37-39, wherein the loading dose of the psychedelic is administered over a period of at or about 1, 2, 3, 4, 5, 6, 7, 8, 9 or 10 minutes, or a range defined by any of the foregoing.

41. The method of any one of claims 37-40, wherein the loading dose of the psychedelic is administered over a period of at or about 3 minutes.

42. The method of any one of claims 38-41, wherein the initial bolus of psychedelic is at a dose of at or about 0.02 mg/kg to at or about 0.2 mg/kg.

43. The method of any one of claims 1-42, wherein the dissociative state is induced by intravenous administration of an initial bolus of the psychedelic at a dose of about 0.1 mg/kg to about 0.2 mg/kg.

44. The method of any one of claims 1, 6, 7, and 9-43, wherein the mean plasma concentration of the psychedelic is maintained at the predetermined value during the therapeutic window by administration of a maintenance dose of the psychedelic.

45. The method of claim 44, wherein the maintenance dose of the psychedelic is administered by continuous or intermittent administration of the psychedelic.

46. The method of claim 45, wherein the continuous or intermittent administration is via an intravenous route.

47. The method of any one of claims 44-46, wherein the maintenance dose is administered by an intravenous infusion.

48. The method of claim 45, wherein the maintenance dose of the psychedelic is administered by intermittent administration of the psychedelic and the intermittent administration is via a subcutaneous, oral, transdermal, intramuscular, intranasal, intranasal/pharanygeal, or buccal route.

49. The method of any one of claims 45-47, wherein the maintenance dose is administered by intravenous infusion of the psychedelic at a rate of at or about 0.2 mg/min to at or about 1 mg/min.

50. The method of any one of claims 45-47, wherein the continuous administration of the psychedelic is at a rate of at or about 0.1 mg/min to at or about 1 mg/min.

51. A method of treating a psychological disorder in a subject, the method comprising:
administering to a subject having a psychological disorder a loading dose of a psychedelic by intravenous administration of an initial bolus in an amount between at or about 1 mg and at or about 5 mg; and
thereafter continuously administering a maintenance dose of the psychedelic by intravenous infusion at a rate of at or about 0.02 mg/min to at or about 1 mg/min to maintain the mean plasma concentration during a therapeutic window; wherein the psychedelic is psilocybin, psilocin, a co-crystal, a co-former, or a salt thereof, or a combination thereof.

52. A method of treating a psychological disorder in a subject, the method comprising:
administering to a subject having a psychological disorder a loading dose of a psychedelic by intravenous infusion at a rate of at or about 0.5 mL/min to at or about 2.0 mL/min over a period of at or about 5 minutes to at or about 20 minutes; and
thereafter continuously administering a maintenance dose of the psychedelic by intravenous infusion at a rate of at or about 0.1 mL/min to at or about 2.0 mL/min over a period of at or about 30 minutes to at or about 120 minutes, to maintain the mean plasma concentration during a therapeutic window; wherein the psychedelic is psilocybin, psilocin, a co-crystal, a co-former, or a salt thereof, or a combination thereof.

53. The method of any one of claims 37-52, wherein the loading dose of the psychedelic is administered at a rate of at or about 0.1 mL/min to at or about 2.0 mL/min.

54. The method of any one of claims 37-53, wherein the loading dose of the psychedelic is administered at a rate of at or about 1.0 mL/min.

55. The method of any one of claims 44-54, wherein the maintenance dose is administered at a rate of 0.1 mL/min to at or about 1.0 mL/min.

56. The method of any one of claims 44-55, wherein the maintenance dose is administered at a rate of at or about 0.5 mL/min.

57. The method of any one of claims 44-56, wherein the maintenance dose is administered over a period of at or about 30, 60, 90 or 120 minutes, or a range defined by any of the foregoing.

58. The method of any one of claims 44-57, wherein the maintenance dose is administered over a period of up to at or about 60 minutes.

59. The method of any one of claims 44-58, wherein the maintenance dose is administered over a period of up to at or about 120 minutes.

60. The method of any one of claims 1, 6, 7, and 9-59, wherein the therapeutic window is between at or about 30 minutes to at or about 120 minutes.

61. The method of any one of claims 1, 6, 7, and 9-60, wherein the therapeutic window is at or about 60 minutes.

62. The method of any one of claims 1, 6, 7, and 9-60, wherein the therapeutic window is at or about 120 minutes.

63. The method of any one of claims 1-62, wherein the total amount of psychedelic that is administered to the subject is up to at or about 1.0 mg, 1.5 mg, 2.5 mg, 5 mg, 7.5 mg, 10 mg, 15 mg or 20 mg per subject, or a range defined by any of the foregoing.

64. The method of any one of claims 1-63, wherein the total amount of psychedelic that is administered to the subject is up to at or about 2.5 mg.

65. The method of any one of claims 1-63, wherein the total amount of psychedelic that is administered to the subject is up to at or about 5 mg.

66. The method of any one of claims 1-63, wherein the total amount of psychedelic that is administered to the subject is up to at or about 10 mg.

67. The method of any one of claims 1-66, wherein the psychedelic is psilocin, a co-crystal, a co-former, or a salt thereof.

68. The method of any one of claims 1-67, wherein the psychedelic is psilocin.

69. The method of any one of claims 1-66, wherein the psychedelic is psilocybin, a co-crystal, a co-former, or a salt thereof.

70. The method of any one of claims 1-66 and 69, wherein the psychedelic is psilocybin.

71. The method of any one of claims 1-68, wherein the psychedelic is psilocin, and the total amount of psilocin that is administered to the subject is up to at or about 2.5 mg, and the administration of psilocin is carried out over a period of at or about 60 minutes.

72. The method of any one of claims 1-66, 69, and 70, wherein the psychedelic is psilocybin, and the total amount of psilocybin that is administered to the subject is up to at or about 5 mg, and the administration of psilocybin is carried out over a period of at or about 60 minutes.

73. The method of any one of claims 1, 2, 6, 7, and 12-72, wherein the predetermined value of mean plasma concentration of the psychedelic is at or about 1-50 μg/L.

74. The method of any one of claims 1, 2, 6, 7, and 12-73, wherein the predetermined value of mean plasma concentration of the psychedelic is at or about 10-20 μg/L.

75. The method of any one of claims 1, 2, 6, 7, and 12-74, wherein the predetermined value of mean plasma concentration of the psychedelic is at or about 10-15 μg/L.

76. The method of any one of claims 1-75, wherein the method further comprises providing psychological support to the subject during the therapeutic window.

77. The method of any one of claims 1-76, wherein the method further comprises withdrawing the subject from the dissociative state at the end of the treatment window by administering a 5HT_{2A} receptor antagonist.

78. The method of any one of claims 1-7 and 9-77, wherein the psychological disorder is selected from the group consisting of PTSD, alcohol addition, drug addiction, treatment resistant depression, anxiety, end of life anxiety, an eating disorder, fibromyalgia, neuropathic pain, phantom limb pain, hypothalamic induced obesity, Prader-Willi syndrome, and binge-eating disorder.

79. The method of any one of claims 1-7 and 9-78, wherein the psychological disorder is selected from the group consisting of hypothalamic induced obesity, Prader-Willi syndrome, binge-eating disorder, and fibromyalgia.

80. The method of any one of claims 1-7 and 9-77, wherein the psychological disorder is a nociplastic pain disorder.

81. The method of any one of claims 1-7 and 9-80, wherein the psychological disorder is fibromyalgia.

82. The method of any one of claims 1-7 and 9-80, wherein the psychological disorder is phantom limb pain.

83. The method of any one of claims 1-7 and 9-80, wherein the psychological disorder is complex regional pain syndrome.

84. The method of any one of claims 1-83, wherein the method further comprises discontinuing the administration if the subject experiences an adverse event.

85. The method of any one of claims 1-84, wherein the psychedelic is administered with at least one additional therapeutic agent.

86. The method of any one of claims 1-85, wherein the method results in the induction of dissociative state in at least 70%, 80%, 90% or 95% of a plurality of subjects that have been administered the psychedelic.

87. The method of any one of claims 1-86, wherein the method results the maintenance of the mean plasma level of the psychedelic in at least 70%, 80%, 90% or 95% of a plurality of subjects that have been administered the psychedelic.

88. The method of any one of claims 1-87, wherein the subject is a human.
